(19) United States
(12) Patent Application Publication
(10) Pub. No.: US 2005/0054826 A1
(43) Pub. Date: Mar. 10, 2005

(54) Human diaphanous-3 gene and methods of use therefor

(75) Inventor: Mao Mao, Redmond, WA (US)

Correspondence Address: JONES DAY, 222 EAST 41ST ST, NEW YORK, NY, 10017, US

(73) Assignee: Rosetta Inpharmatics LLC

(21) Appl. No.: 10/848,755

(22) Filed: May 18, 2004

Related U.S. Application Data
(60) Provisional application No. 60/471,842, filed on May 19, 2003.

(51) Int. Cl. C07K 14/705; C07H 21/04

(52) U.S. Cl. 530/350; 536/23.5; 435/69.1; 435/320.1; 435/325

(57) ABSTRACT

The present invention is directed to the full-length cDNA sequence encoding human diaphanous-3 (DIAPH3), to DIAPH3 encoded thereby, and to fragments of DIAPH3 and the cDNA. The present invention also provides for the use of the cDNA, and of DIAPH3, as a marker of poor prognosis of breast cancer. Because DIAPH3 appears essential for proper spindle pole formation during mitosis, DIAPH3 is a useful target for screening assays designed to identify inhibitors or modulators of DIAPH3 activity, which are useful for the treatment of cancer, particularly breast cancer. Thus, the invention further provides methods of using DIAPH3, or fragments thereof, in assays to identify such compounds.

This application claims benefit of U.S. Provisional Application Ser. No. 60/471,842, filed May 19, 2003, which is hereby incorporated by reference herein in its entirety.

This application includes a Sequence Listing submitted on compact disc, recorded on two compact discs, including one duplicate, containing Filename 9301196999.txt, of size 622,060 bytes, created May 14, 2004. The sequence listing on the compact discs is incorporated by reference herein in its entirety.

### 1. FIELD OF THE INVENTION

The present invention relates to the identification of the full-length sequence of a human breast cancer-related cDNA referred to herein as DIAPH3. The invention specifically relates to the nucleotide sequence of the DIAPH3 cDNA, and subsequences thereof, and to the encoded DIAPH3 protein and analogs thereof. The invention further relates to the use of the DIAPH3 cDNA in the prognosis of breast cancer. The invention also relates to the use of the DIAPH3 cDNA, the coding sequences thereof, or the DIAPH3 protein as a target for anti-cancer drugs, and in methods for the identification of molecules that have anti-cancer activity.

### 2. BACKGROUND OF THE INVENTION

### 2.1 Breast Cancer

The increased number of cancer cases reported in the United States, and, indeed, around the world, is a major concern. Currently there is only a handful of treatments available for specific types of cancer, and these provide no guarantee of success. In order to be most effective, these treatments require not only an early detection of the malignancy, but a reliable assessment of the severity of the malignancy.

The incidence of breast cancer, a leading cause of death in women, has been gradually increasing in the United States over the last thirty years. Its cumulative risk is relatively high; 1 in 8 women are expected to develop some type of breast cancer by age 85 in the United States. In fact, breast cancer is the most common cancer in women and the second most common cause of cancer death in the United States. In 1997, it was estimated that 181,000 new cases were reported in the U.S., and that 44,000 people would die of breast cancer (Parker et al., CA Cancer J. Clin. 47:5-27 (1997); Chu et al., J. Nat. Cancer Inst. 88:1571-1579 (1996)). While the mechanism of tumorigenesis for most breast carcinomas is largely unknown, there are genetic factors that can predispose some women to developing breast cancer (Miki et al., Science, 266:66-71(1994)). The discovery and characterization of BRCA1 and BRCA2 has recently expanded our knowledge of genetic factors which can contribute to familial breast cancer. Germ-line mutations within these two loci are associated with a 50 to 85% lifetime risk of breast and/or ovarian cancer (Casey, Curr. Opin. Oncol. 9:88-93 (1997); Marcus et al., Cancer 77:697-709 (1996)). Only about 5% to 10% of breast cancers are associated with breast cancer susceptibility genes, BRCA1 and BRCA2. The cumulative lifetime risk of breast cancer for women who carry the mutant BRCA1 is predicted to be approximately 92%, while the cumulative lifetime risk for the non-carrier majority is estimated to be approximately 10%. BRCA1 is a tumor suppressor gene that is involved in DNA repair and cell cycle control, which are both important for the maintenance of genomic stability. More than 90% of all mutations reported so far result in a premature truncation of the protein product with abnormal or abolished function. The histology of breast cancer in BRCA1 mutation carriers differs from that in sporadic cases, but mutation analysis is the only way to find the carrier. Like BRCA1, BRCA2 is involved in the development of breast cancer, and like BRCA1 plays a role in DNA repair. However, unlike BRCA1, it is not involved in ovarian cancer.

Other genes have been linked to breast cancer, for example c-erb-2 (HER2) and p53 (Beenken et al., Ann. Surg. 233(5):630-638 (2001). Overexpression of c-erb-2 (HER2) and p53 have been correlated with poor prognosis (Rudolph et al., Hum. Pathol. 32(3):311-319 (2001), as has been aberrant expression products of mdm2 (Lukas et al., Cancer Res. 61(7):3212-3219 (2001) and cyclin1 and p27 (Porter & Roberts, International Publication WO98/33450, published Aug. 6, 1998). However, no other clinically useful markers consistently associated with breast cancer have been identified.

Sporadic tumors, those not currently associated with a known germline mutation, constitute the majority of breast cancers. It is also likely that other, non-genetic factors also have a significant effect on the etiology of the disease. Regardless of the cancer's origin, breast cancer morbidity and mortality increases significantly if it is not detected early in its progression. Thus, considerable effort has focused on the early detection of cellular transformation and tumor formation in breast tissue, and the nucleotide sequences of breast cancer-related genes or the cDNAs derived therefrom. The present application provides one such sequence.

### 2.2 Diaphanous Proteins and Tumorigenesis

The misregulation of genes associated with cell-cycle control and cytoskeletal restructuring have been implicated in the etiology of various cancers.

A group of small GTP-binding proteins (G-proteins) with molecular weights of 20,000-30,000 with no subunit structure has been observed in various organisms. To date, over fifty or more members have been found as the superfamily of the small G-proteins in a variety of organisms, from yeast to mammals. The group of small G-proteins includes the Rho protein, which is considered to control cell morphological change, adhesion and motility. When the inactive GDP-binding Rho is stimulated, it is transformed to the active GTP-binding Rho protein by GDP/GTP exchange proteins such as Smg GDS, Dbl or Ost. The activated Rho protein then acts on target proteins to form stress fibers and focal contacts, thus inducing the cell adhesion and motility (Takai et al., Trends Biochem. Sci., 20:227-231 (1995)). Rho is also considered to be implicated in physiological functions associated with cytoskeletal rearrangements, such as cell morphological change (Parterson et al., J. Cell Biol., 111:1001-1007 (1990)), cell adhesion (Morii et al., J. Biol. Chem. 267:20921-20926 (1992); Tominaga et al., J. Cell Biol. 120:1529-1537 (1993); Nusrat et al., Proc. Natl. Acad. Sci. U.S.A. 92:10629-10633 (1995); Landanna et al., Science 271:981-983 (1996)), cell motility (Takaishi et al., Oncogene 9:273-279 (1994)); cytokinesis (Kishi et al., J. Cell Biol. 120:1187-1195 (1993); and metastasis (Yoshioka et al., FEBS Lett., 372:25-28 (1995)). Rho exerts its effects on the actin cytoskeleton, which plays an important role in cell motility, morphology, phagocytosis and cytokinesis.

Formin homology domain proteins have also been implicated in the control of rearrangements of the actin cytoskeleton, especially in the context of cytokinesis and cell polarization. See Ridley, Nature Cell Biol. 1:E64-E66 (1999). Members of this family have been shown to interact with Rho-GTPases (Alberts, J. Biol. Chem. 276(4):2824-2830 (2001); Tominaga et al., Mol. Cell 5:13-25 (2000)), profilin, and other actin-associated proteins. These interactions are mediated by a proline-rich FH1 domain, usually located in front of the FH2 domain.

One group of formin homology domain proteins, related to the D. melanogaster Diaphanous protein, have been identified in mouse and in humans. The murine homolog of Diaphanous, Dia, interacts with Rho GTPase to effect cytoskeletal rearrangements. See U.S. Pat. No. 6,111,072. In mouse, a variant of the gene dia, showing limited nucleotide sequence homology to the D. melanogaster dia gene, has been shown to be expressed in osteosarcoma cells. See Fukuda et al., Biochem. Biophys. Res. Comm. 261(1):35-40 (1999)).

In humans, two dia-like genes have been identified. The gene encoding the FH protein DIA has been implicated in premature ovarian failure (Bione et al., Am. J. Hum. Genet. 62:533-541 (1998)), and the related DFNA1 gene has been implicated in nonsyndromic deafness in a large Costa Rican kindred (Lynch et al., Science 278:1315-1318 (1997); see also U.S. Pat. No. 6,197,932; U.S. Pat. No. 5,985,574; U.S. Pat. No. 6,111,072). The DIAPH3 sequence described herein, and the DIAPH3 protein encoded thereby, constitute a third class of human dia-like sequence. Prior to the present invention, no connection had been demonstrated in humans between a diaphanous-like protein and breast cancer.

### 3. SUMMARY OF THE INVENTION

The present invention provides a DIAPH3 protein and fragments thereof. In one embodiment, the invention provides a purified protein comprising the C-terminal 60 contiguous amino acids of SEQ ID NO: 3, wherein said purified protein displays the antigenicity or immunogenicity of SEQ ID NO: 3. In a specific embodiment, said protein comprises the C-terminal 500 amino acids of SEQ ID NO: 3. In another specific embodiment, said protein comprises SEQ ID NO: 3. In another specific embodiment, said protein comprises amino acids 636-1110 of SEQ ID NO: 3. In another specific embodiment, said purified protein consists of less than the entire amino acid sequence of SEQ ID NO: 3.

The invention also provides DIAPH3-encoding nucleic acids and fragments thereof. Thus, in another embodiment, the invention provides an isolated nucleic acid comprising 3750 contiguous nucleotides of SEQ ID NO: 1, or the complement thereof. In specific embodiment, said isolated nucleic acid comprises 500 contiguous nucleotides of the 3′ end of SEQ ID NO: 1, or the complement thereof. In another specific embodiment, said isolated nucleic acid comprises the nucleotide sequence of SEQ ID NO: 1, or the complement thereof. In another specific embodiment, the isolated nucleic acid is DNA. In another embodiment, the invention provides an isolated nucleic acid comprising a nucleotide sequence encoding a protein the amino acid sequence of which consists of SEQ ID NO: 3, or a protein comprising the C-terminal contiguous amino acids of SEQ ID NO: 3, wherein said protein displays the antigenicity or immunogenicity of SEQ ID NO: 3, or the complement of said nucleotide sequence. In another embodiment, the invention provides a cell transformed with a nucleic acid, said nucleic acid comprising (a) a nucleotide sequence encoding a protein comprising the C-terminal 100 contiguous amino acids of SEQ ID NO: 3, wherein said protein displays the antigenicity or immunogenicity of SEQ ID NO: 3, or (b) the complement of said nucleotide sequence. In another embodiment, the invention provides a recombinant cell containing a nucleic acid comprising 3750 contiguous nucleotides of SEQ ID NO: 1, or the complement thereof, in which the nucleotide sequence is under the control of a promoter heterologous to the nucleotide sequence. In a specific embodiment, this nucleic acid is contained within a vector.

The invention also provides antibodies to a DIAPH3 protein or fragments thereof. In one embodiment, the invention provides an antibody that specifically binds to a protein the amino acid sequence of which consists of SEQ ID NO: 3. In specific embodiment, said antibody is monoclonal. In another embodiment, the invention provides a molecule comprising a fragment of the antibody of claim 14, which fragment binds said protein. In another embodiment, said antibody specifically binds an epitope present in amino acids 1110-1152 of SEQ ID NO: 3.

The invention further provides a method of producing a protein comprising growing a recombinant cell containing a nucleic acid that encodes a protein comprising SEQ ID NO: 3, or a protein comprising the C-terminal 100 contiguous amino acids of SEQ ID NO: 3, in which said nucleotide sequence is under the control of a promoter heterologous to said nucleotide sequence, such that the protein encoded by said nucleic acid is expressed by the cell; and recovering said expressed protein. The invention also provides an isolated protein that is the product of this method.

The invention further provides pharmaceutical composition comprising a therapeutically effective amount of a purified protein comprising SEQ ID NO: 3, or a protein comprising the C-terminal 100 contiguous amino acids of SEQ ID NO: 3, and a pharmaceutically acceptable carrier. In another embodiment, the invention provides a pharmaceutical composition comprising a therapeutically effective amount of the nucleic acid comprising 3750 contiguous nucleotides of SEQ ID NO: 1, or a nucleic acid encoding a protein comprising SEQ ID NO: 3, or a protein comprising the C-terminal 100 contiguous amino acids of SEQ ID NO: 3; and a pharmaceutically acceptable carrier. In another embodiment, the invention provides a pharmaceutical composition comprising a therapeutically effective amount of an antibody that specifically binds to a protein the amino acid sequence of which that consists of SEQ ID NO: 3, or specifically binds to an epitope present in amino acids 1110-1152 of SEQ ID NO: 3, and a pharmaceutically acceptable carrier.

The invention further provides a method of identifying an agent that modulates the binding of a protein comprising SEQ ID NO: 3 to a binding partner, comprising contacting said protein and said binding partner with an agent; and measuring an amount of a complex comprising said protein and said binding partner in the presence of said agent, wherein if said amount differs from said amount in the absence of said agent, said agent is identified as an agent that modulates the binding of said protein to said binding partner. In a specific embodiment, said protein comprising SEQ ID NO: 3 is purified. In a specific embodiment, said agent, or said binding partner is purified. The invention further provides a method of identifying a molecule that binds to a ligand, comprising: (a) contacting a ligand with one or more candidate binding molecules under conditions conducive to binding between said ligand and said molecules, wherein said ligand is selected from the group consisting of a first protein comprising SEQ ID NO: 3, a second protein comprising a fragment of SEQ ID NO: 3 comprising the FH2 domain of DIAPH3 but less than all of SEQ ID NO: 3, and a nucleic acid encoding said first protein or said second protein; and (b) identifying any of said molecules that specifically binds to said ligand. In a specific embodiment, said first protein or said second protein is purified. In a specific embodiment, said molecule is an antibody or a small molecule.

The present invention further provides methods of diagnosis and prognosis of breast cancer using the nucleic acids, proteins or antibodies of the invention. In one embodiment, the invention provides a method of diagnosing an individual as having breast cancer, comprising comparing the level of expression of a nucleic acid encoding SEQ ID NO: 3 in a sample derived from breast cells of said individual to a control level of said expression, and diagnosing said individual as having breast cancer if said level of expression of said nucleic acid encoding SEQ ID NO: 3 is higher than said control level of expression. In a specific embodiment, said level of expression of a nucleic acid encoding SEQ ID NO: 3 is determined by hybridizing said nucleic acid with an oligonucleotide complementary and hybridizable to nucleotides 1-862, 2927-3045, or 3412-3929 of SEQ ID NO: 1, and determining the amount of said hybridization. In another embodiment, the invention provides a method of diagnosing an individual as having breast cancer comprising comparing the level of a protein the amino acid sequence of which consists of SEQ ID NO: 3 in a sample derived from breast cells of said individual to a control level of said protein; and classifying said individual as having breast cancer if said level of said protein in said sample is higher than said control level of said protein. The invention also provides a method of imaging a breast cancer tumor comprising: (a) contacting cells of said tumor with an antibody that binds specifically to a protein the amino acid sequence of which consists of SEQ ID NO: 3, wherein said antibody is labeled; and (b) detecting said label. The invention further provides a method of predicting the prognosis of a breast cancer patient comprising: (a) determining the level of expression of a nucleic acid encoding SEQ ID NO: 3 in a sample derived from breast cancer tumor cells from said patient; (b) comparing said level of expression to a control level of said expression; and (c) predicting that said patient will have a poor prognosis if said level of expression of said nucleic acid encoding SEQ ID NO: 3 in said sample is higher than said control level of said expression. In a specific embodiment, said level of expression of a nucleic acid encoding SEQ ID NO: 3 is determined by hybridizing said nucleic acid with an oligonucleotide complementary and hybridizable to nucleotides 1-862, 2927-3045, or 3412-3929 of SEQ ID NO: 1, and determining the amount of said hybridization. In another specific embodiment, said determining is carried out by a method comprising: (a) hybridizing nucleic acids in said sample to an oligonucleotide, wherein said oligonucleotide is hybridizable to SEQ ID NO: 1 or its complement; and (b) determining the amount of said hybridization. In a more specific embodiment, said oligonucleotide is a probe on a microarray. In another more specific embodiment, said oligonucleotide is one of a plurality of probes on a microarray, wherein said plurality comprises probes complementary and hybridizable to nucleic acids respectively encoded by five different breast cancer-related markers that do not encode SEQ ID NO: 3. In another more specific embodiment, said oligonucleotide is one of a plurality of probes on a microarray, wherein said plurality comprises probes complementary and hybridizable to nucleic acids respectively encoded by twenty different breast cancer-related markers that do not encode SEQ ID NO: 3. In an even more specific embodiment, said five different breast cancer-related markers are present in Table 1. In another even more specific embodiment, said five different breast cancer-related markers are present in Table 2. The invention also provides a method of predicting the prognosis of a breast cancer patient comprising: (a) determining the level of a protein comprising SEQ ID NO: 3 in a sample derived from breast cancer tumor cells from said patient; (b) comparing said level of said protein to a control level of said protein; and (c) predicting that said patient will have a poor prognosis if said level of said protein comprising SEQ ID NO: 3 is significantly higher than said control level of said protein. In a specific embodiment, said determining is carried out by a method comprising: (a) contacting said protein comprising SEQ ID NO: 3 from said sample with an antibody that specifically binds said protein; and (b) determining the amount of antibody bound to said protein, wherein said amount of antibody bound to said protein indicates said level of said protein in said breast cancer tumor sample.

The present invention also provides kits useful for the detection, diagnosis and/or prognosis of breast cancer. In one embodiment, the invention provides a kit comprising in a first container an oligonucleotide that hybridizes to SEQ ID NO: 1 under stringent conditions, wherein said oligonucleotide is at least 12 nucleotides in length, and wherein said oligonucleotide is complementary and hybridizable to nucleotides 1-862, 2927-3045, or 3412-3929 of SEQ ID NO: 1. In another embodiment, the invention provides a kit for the diagnosis and/or prognosis of breast cancer, comprising in a first container an oligonucleotide that hybridizes to a nucleotide sequence that encodes SEQ ID NO: 3 under stringent conditions, wherein said oligonucleotide is at least 12 nucleotides in length, and wherein said oligonucleotide is complementary and hybridizable to nucleotides 1-862, 2927-3045, or 3412-3929 of SEQ ID NO: 1, and further comprising in a second container a known amount of a nucleic acid to which said oligonucleotide is complementary and hybridizable. In a specific embodiment, said oligonucleotide is a probe on a microarray. In a more specific embodiment, said microarray comprises probes complementary and hybridizable to nucleic acids respectively encoded by breast cancer-related markers other than a nucleotide sequence that encodes SEQ ID NO: 3. The invention also provides an article of manufacture comprising a container comprising a purified protein comprising SEQ ID NO: 3. The invention further provides a kit comprising in a first container an antibody that specifically binds to a protein the amino acid sequence of which consists of SEQ ID NO: 3, or binds specifically to a fragment of said protein, and further comprising in a second container a known amount of said protein or a fragment thereof to which said antibody binds. In a specific embodiment, said antibody specifically binds an epitope present in amino acids 1110-1152 of SEQ ID NO: 3. In another embodiment, the invention provides a kit comprising in one or more containers a forward primer and a reverse primer that amplify at least a portion of the nucleotide sequence of SEQ ID NO: 1 when used in a polymerase chain reaction, wherein said forward primer and said reverse primer are complementary and hybridizable to nucleotides 1-862, 2927-3045, or 3412-3929 of SEQ ID NO: 1 or the complementary sequence thereof.

The invention also provides a method of inhibiting the expression of a nucleotide sequence encoding SEQ ID NO: 3 comprising contacting an RNA encoding SEQ ID NO: 3 with an interfering RNA, said interfering RNA comprising a nucleotide sequence complementary and hybridizable to SEQ ID NO: 1, under conditions that allow said interfering RNA and said mRNA to hybridize. In a specific embodiment, said nucleotide sequence of said interfering RNA, or a complement thereof, is present within nucleotides 1-862, 2927-3045, or 3412-3929 of SEQ ID NO: 1. In another specific embodiment, said nucleotide sequence of said interfering RNA is selected from the group consisting of SEQ ID NO: 274 and SEQ ID NO: 275.

### 3.1 Definitions

As used herein, italicization indicates a nucleotide sequence such as a gene or cDNA sequence and roman type indicates the encoded protein or polypeptide. For example, “DIAPH3” shall mean a cDNA, or the gene from which the cDNA is derived, encoding the protein product “DIAPH3.” “DIAPH3” and DIAPH3 refer not only to the human nucleotide sequence and protein, respectively, but to homologs of each from other species.

“Breast cell” as used herein indicates any cell normally associated with the breast, or which the breast comprises, including epithelial and endothelial cells, fat cells, duct cells, etc.

“Protein” as used herein includes peptides and polypeptides.

### 4. BRIEF DESCRIPTION OF THE DRAWINGS

FIGS. 1A-1B depict the full-length sequence of the 4331 nucleotide DIAPH3 cDNA (SEQ ID NO: 1).

FIG. 2A-2C depict the coding region (SEQ ID NO: 2) of the DIAPH3 cDNA sequence aligned to the amino acid sequence of the predicted DIAPH3 protein product (SEQ ID NO: 3) encoded thereby. The nucleotide sequence of SEQ ID NO: 2 is nucleotides 93-3551 of SEQ ID NO: 1.

FIG. 3 depicts the UCSC linkage map of a region of chromosome 13q21.2 containing poor breast cancer prognosis markers AL137718, Contig28552 and Contig46218 (University of California-Santa Cruz, April, 2002 freeze). Specific features presented in the linkage map are as follows. “Base Position”: Chromosomal coordinates, numbered from the telomere of the short arm of human chromosome 13. “Chromosome Band”: Light and dark blocks show traditional cytological bands seen with Giemsa staining. STS Markers: Location of markers from genetic, RH, YAC, and FISH maps. “Gap”: Shows locations of gaps in the assembly with black boxes or vertical lines. Small gaps may have artefactually coalesced in the graphic. Gaps spanned by mRNA and paired reads have a white horizontal line through the black box to indicate bridging. “Coverage”: In dense display, the level of gray gives level of coverage: White/Clear: no coverage (gap); Light Gray: predraft (less than 4× shotgun); Medium Gray: draft (at least 4× shotgun); Dark Gray: multiple draft, covered by more than one draft clone; Finished: covered by a finished clone. “YourSeq”: Position of the query DNA sequence relative to other sequences or features in the linkage map. “Known Genes (from RefSeq)”: Known protein-coding genes from LocusLink. Exons are represented by black boxes; thin horizontal lines represent introns. In the full view, the arrows on the introns indicate direction of transcription. “Acembly Gene Predictions with Alt Splicing”: Gene models reconstructed solely from mRNA and EST evidence by Danielle and Jean Thierry-Mieg and Vahan Simonyan using the Acembly program. “Genscan Gene Predictions”: Gene predictions using the program Genscan, which uses predictions are based on transcriptional, translational, and donor and acceptor splicing signals, plus length and compositional distributions of exons, introns and intergenic regions. “Human mRNAs from Genbank”: Alignments between human mRNAs in Genbank and the genome using the BLAT program. “Human ESTs That Have Been Spliced”: Alignments between spliced Expressed Sequence Tags (ESTs) in Genbank and the genome using the BLAT program. “Nonhuman mRNAs from Genbank”: Translated BLAT alignments of non-human vertebrate mRNA from Genbank. “Overlap SNPs”: Single nucleotide polymorphisms found on overlapping contigs. “Random SNPs”: Displays single nucleotide polymorphisms (SNPs) found by random sequencing. “RepeatMasker”: Shows dispersed repeats as determined by RepeatMasker using the Repbase Update library of repetitive sequences from the Genetic Information Research Institute. These elements include SINE, LINE, LTR, DNA, simple, low complexity, micro-satellite, tRNA, and other repeat families.

FIG. 4 depicts array data demonstrating that the expression of DIAPH3 clusters with, or is co-regulated with, the expression of other genes associated with mitosis-related genes.

FIG. 5 depicts the percentage of living cells present after treatment with DIAPH3-derived small interfering RNAs (siRNAs) DIAPH3-1555 or DIAPH3-1805, as compared to an siRNA for luciferase. Cells were transfected with a luciferase siRNA, DIAPH3-1555 or DIAPH3-1805, or were mock-transfected, grown for 72 hours, and stained with crystal violet.

FIGS. 6A-6C depict experiments demonstrating the effect of disruption of DIAPH3 expression on mitotic spindle pole formation. FIG. 6A depicts a mock-treated HeLa cell in mitosis, showing normal dipolar mitotic spindle formation. FIG. 6B depicts aberrant tripolar (top) and quadripolar (bottom) mitotic spindle formation when HeLa cells are transfected with the siRNA DIAPH3-1555. FIG. 6C depicts aberrant tripolar (top) and quadripolar (bottom) mitotic spindle formation when HeLa cells are transfected with the siRNA DIAPH3-1803.

FIG. 7 depicts results of experiments to determine the percentage of mitotic HeLa cells displaying aberrant mitotic spindle formation, where the cells were transfected with a luciferase siRNA, the siRNAs DIAPH3-1555 or DIAPH3-1805, or were mock-transfected. Percentages indicate the percent of cells showing aberrant spindle formation out of all cells in culture identified as mitotic.

FIGS. 8A-8C depict light micrographs demonstrating multinucleation resulting from disruption of DIAPH3 expression. FIG. 7A depicts mock-transfected HeLa cells that are normally nucleated. FIG. 7B depicts HeLa cells transfected with DIAPH3-1555. The cells display an abnormal, multinucleate physiology. FIG. 7C depicts HeLa cells transfected with DIAPH3-1805. The cells display an abnormal, multinucleate physiology.

FIG. 9 depicts the percentages of cells showing micronucleation or multinucleation resulting from transfection with DIAPH3 siRNAs DIAPH3-1555 or DIAPH3-1805. The percentage of cells, indicated on the Y-axis, is the percentage of cells counted that display multinucleation (light gray bars) or micronucleation (dark gray bars).

### 5. DETAILED DESCRIPTION OF THE INVENTION

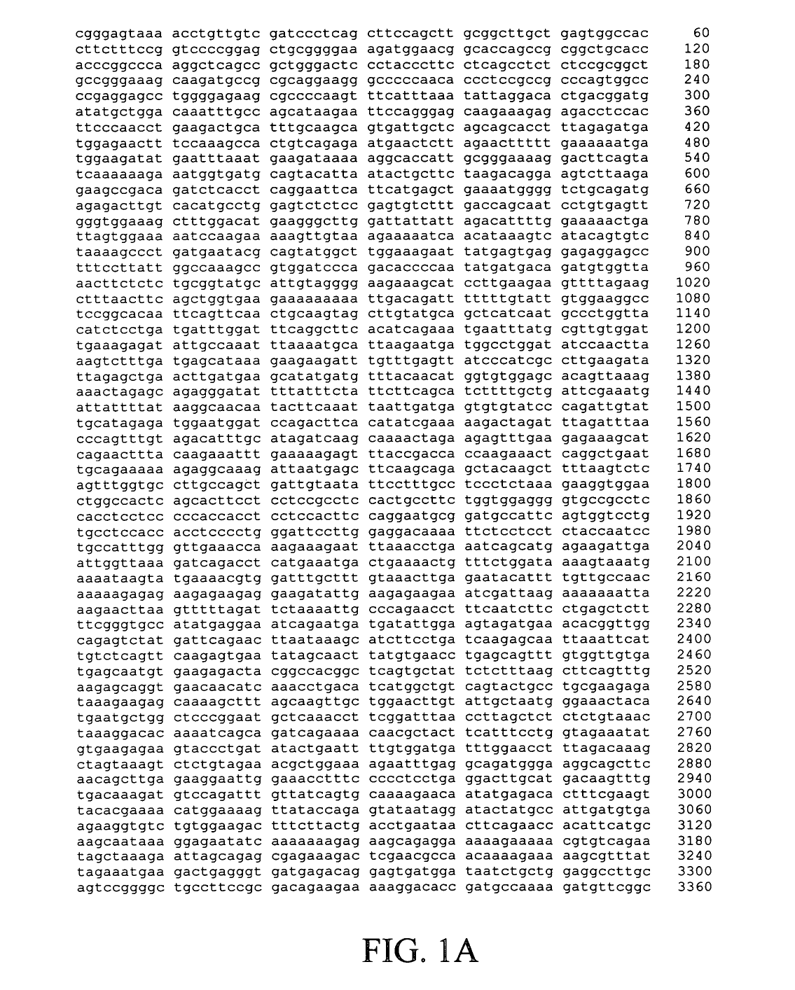

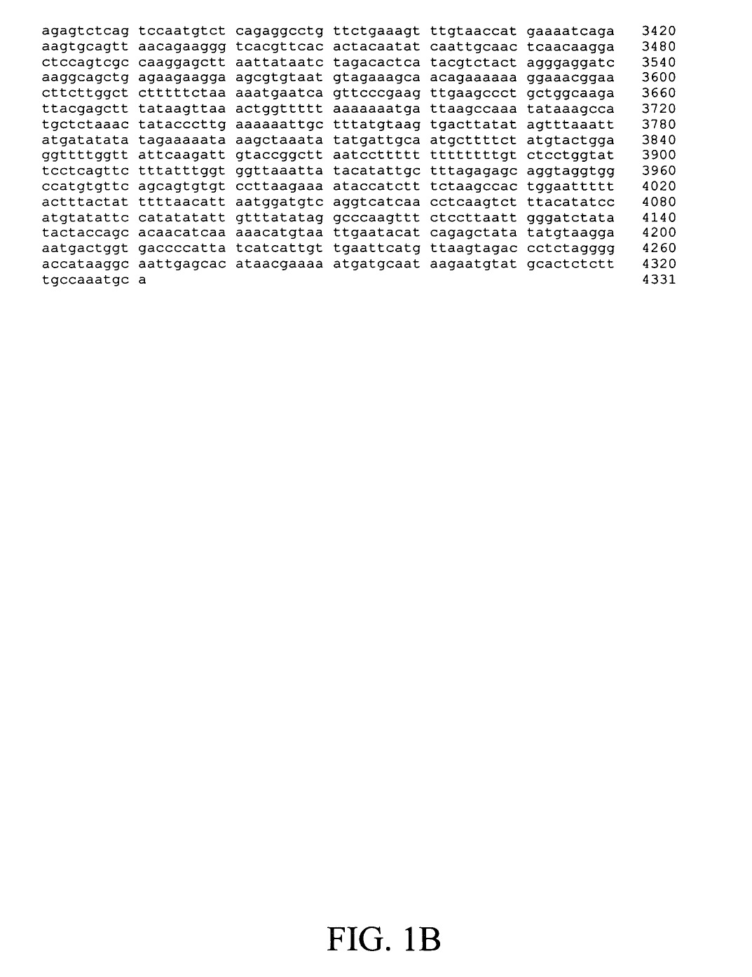

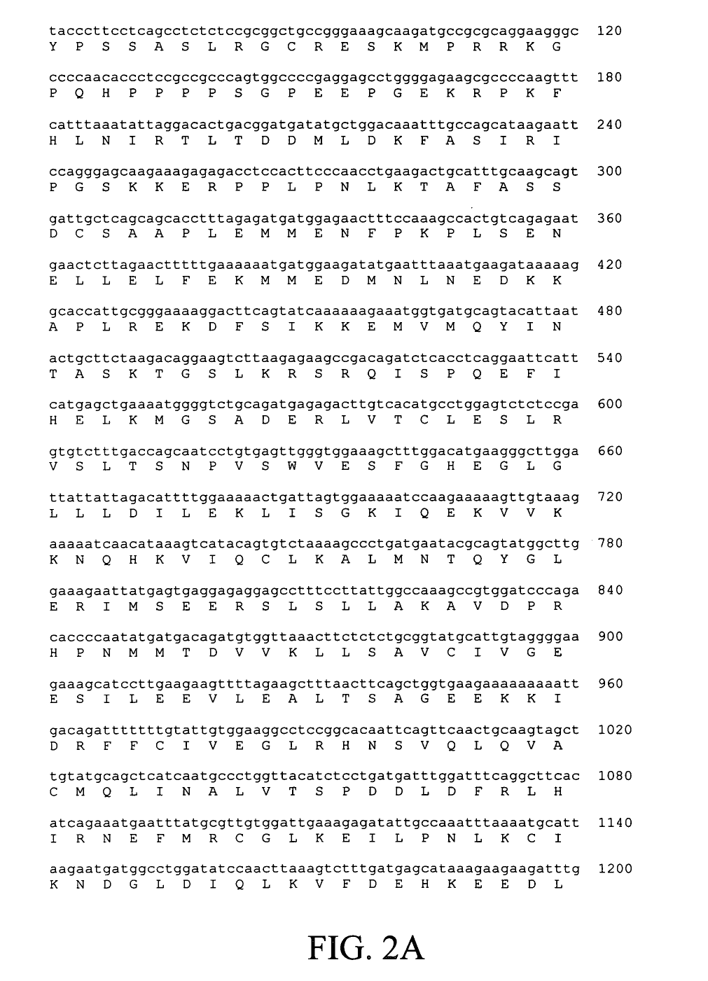

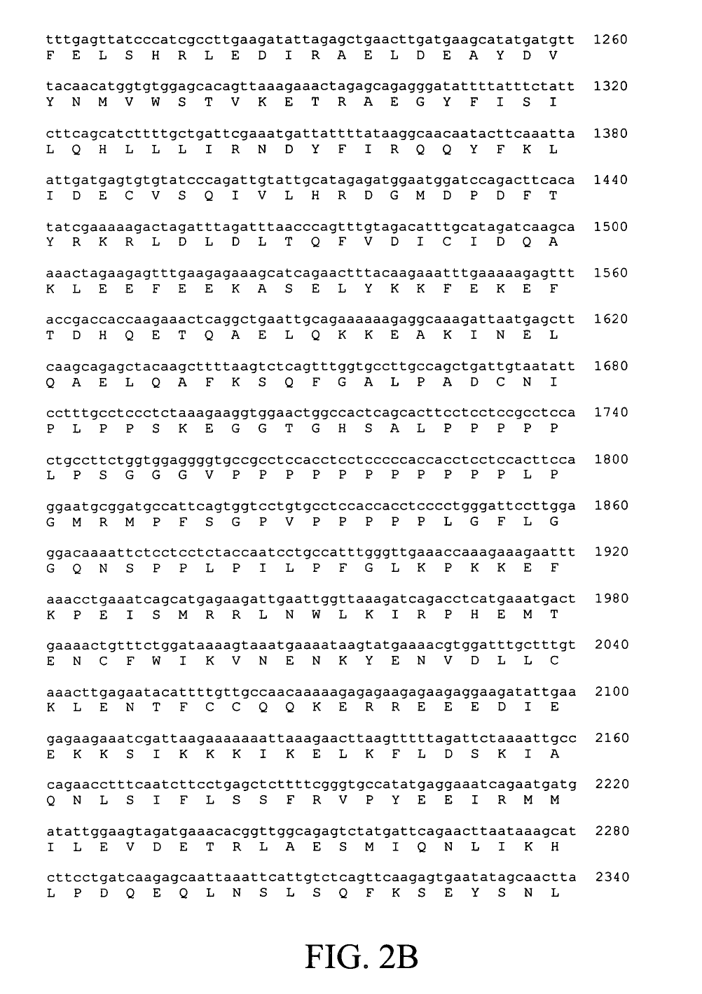

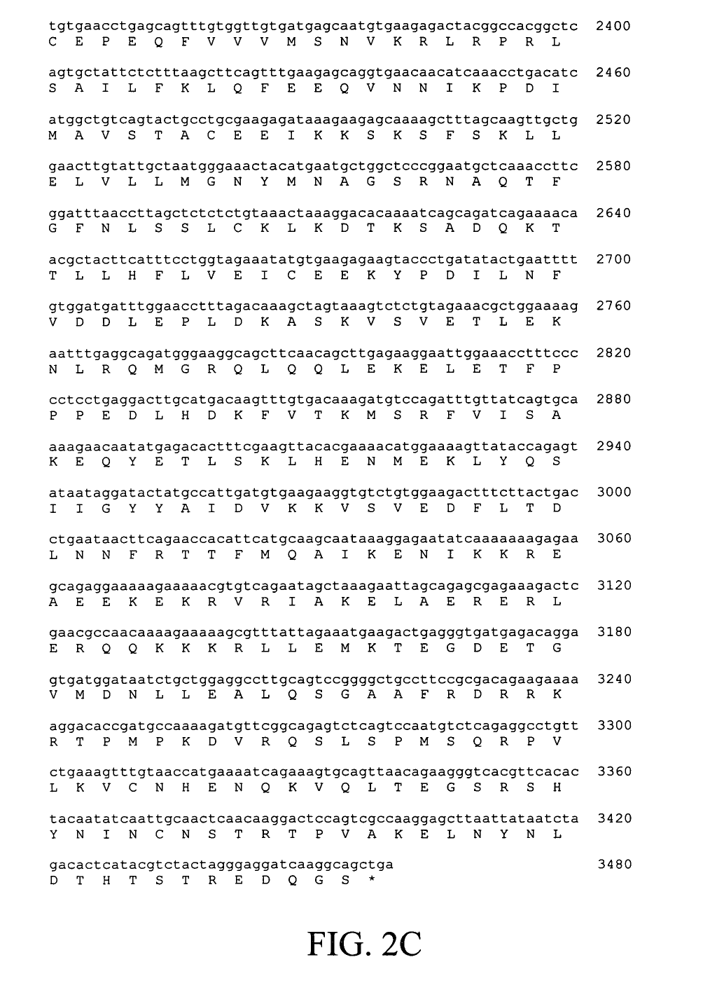

The present invention relates to the full-length human DIAPH3 cDNA and the DIAPH3 protein encoded thereby. SEQ ID NO: 1 is the full-length DIAPH3 cDNA sequence (FIG. 1), which includes the DIAPH3 coding sequence (SEQ ID NO: 2: FIG. 2) that encodes the DIAPH3 protein (SEQ ID NO: 3: FIG. 2). DIAPH3 is a formin homology domain (FH) protein, and is predicted to contain an FH2 domain between amino acid residues 636 and 1077, inclusive.

### 5.1 Isolation of DIAPH3 and DIAPH3-Related Genes

The invention first relates to the nucleotide sequence of DIAPH3. In a specific embodiment, the invention relates to the full-length DIAPH3 cDNA as presented in FIG. 1 (SEQ ID NO: 1). In another specific embodiment, the invention provides the coding or cDNA sequence of the DIAPH3 gene (FIG. 2; SEQ ID NO: 2) and the encoded DIAPH3 protein (FIG. 2; SEQ ID NO: 3). The nucleotide sequence of SEQ ID NO: 2 is nucleotides 93-3551 of SEQ ID NO: 1.

The invention provides purified nucleic acids consisting of at least 10 nucleotides (i.e., a hybridizable portion) of a nucleotide sequence encoding DIAPH3; in other embodiments, the nucleic acids consist of at least 10, 20, 50, 100, 150, 200, 300, 400, 500, 600, 700, 800, 900, 100, 1100, 1200, 1500, 2000, 2300, 2500, 3000, 3250, 3500, 3750 or 4000 contiguous nucleotides of a nucleotide sequence encoding DIAPH3. In another embodiment, the nucleic acids consist of at least the 10, 20, 50, 100, 150, 200, 300, 400, 500, 600, 700, 800, 900, 100, 1100, 1200, 1500, 2000, 2300, 2500, 3000, 3250, 3500, 3750 or 4000 contiguous nucleotides of the 3′ end of the nucleotide sequence of SEQ ID NO: 1. In another embodiment, the nucleic acids are smaller than 35, 200 or 500 nucleotides in length. Nucleic acids can be single or double stranded. In another embodiment, the nucleic acids comprise a sequence of at least 10 nucleotides that encode a fragment of DIAPH3, wherein the fragment of DIAPH3 displays one or more functional activities of DIAPH3, or contains a functional domain or motif of DIAPH3. In no event, however, does the invention provide for a contiguous nucleic acid sequence wholly contained within the sequence depicted in Genbank Accession No. AL137718, Contig28552 or Contig46218 (see Example 1).

The invention also relates to nucleic acids hybridizable to or complementary to the foregoing sequences. In specific aspects, nucleic acids are provided which comprise a sequence complementary to at least 20, 30, 40, 50, 100, or 200 nucleotides or the entire coding region of DIAPH3, or the reverse complement (antisense) of any of these sequences. In a specific embodiment, a nucleic acid which is hybridizable to DIAPH3 (e.g., having part or the whole of sequence SEQ ID NO: 1 or SEQ ID NO: 2, or the complement thereof), or to a nucleic acid encoding a DIAPH3 derivative, under conditions of low stringency is provided. By way of example and not limitation, procedures using such conditions of low stringency are as follows (see also Shilo and Weinberg, Proc. Natl. Acad. Sci. U.S.A. 78:6789-6792 (1981)): Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg g/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and re-exposed to film. Other conditions of low stringency which may be used are well known in the art (e.g., as employed for cross-species hybridizations).

In another specific embodiment, a nucleic acid hybridizable to a nucleic acid encoding DIAPH3, or its reverse complement, under conditions of high stringency is provided. By way of example and not limitation, procedures using such conditions of high stringency are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500:g/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Other conditions of high stringency that may be used are well known in the art. Nucleic acids hybridizable to the complement of the above-mentioned sequences are also provided.

The above-mentioned nucleic acids preferably also encode a protein displaying one or more functional activities of DIAPH3 or a domain or motif thereof.

Nucleic acids encoding derivatives of DIAPH3 (see Section 5.6), and antisense nucleic acids to sequences encoding DIAPH3 (see Section 5.9.2) are additionally provided. As is readily apparent, as used herein, a nucleic acid encoding a “fragment” or “portion” of DIAPH3 shall be construed as referring to a nucleic acid encoding only the recited fragment or portion of DIAPH3 and not the other contiguous portions of DIAPH3 as a continuous sequence.

Fragments of nucleic acids encoding DIAPH3, which comprise regions conserved between (i.e., having homology or identity to) other DIAPH3-encoding nucleic acids of the same or different species, are also provided. Nucleic acids encoding one or more domains of DIAPH3 are provided.

Fragments or derivatives of DIAPH3 that hybridize specifically to DIAPH3, and thus can be used as hybridization probes in hybridization assays to detect upregulation or downregulation of DIAPH3, are also provided. In such embodiments, oligonucleotides of at least 10, 15, 20, 25, 30, 35, 40, 45, 50, 60, 70, 80, 90 or 100 nucleotides are provided. In specific embodiments, oligonucleotides, preferably oligodeoxyribonucleotides, in the range of 10-100, 15-80, or 40-70 nucleotides are provided as hybridization probes. Oligoribonucleotides that hybridize specifically to DIAPH3 are also provided in the invention.

The invention also provides nucleic acids comprising nucleotide sequences of at least 60, 70, 90, 95 or 99% homologous to a nucleotide sequence of DIAPH3 or a portion thereof. “Homologous” means that in various embodiments, the aligned first nucleotide sequence has preferably at least 30% or 50%, more preferably 60% or 70%, even more preferably at least 80% or 90%, and even more preferably at least 95% identity to a second nucleotide sequence over a nucleotide sequence length equal to the shorter of the two sequences, plus any introduced gaps. When the alignment is done by a computer homology program known in the art, such as BLAST (blastn), the percent homology is calculated by dividing the number of nucleotides in the DIAPH3-encoding nucleic acid sequence or fragment thereof exactly matching the nucleotide at the same position in the aligned sequence by the length of the alignment in nucleotides, including introduced gaps, where introduced gaps count as mismatches.

Specific embodiments for the cloning of a gene or cDNA encoding DIAPH3, presented as a particular example but not by way of limitation, follows:

For expression cloning (a technique commonly known in the art), an expression library is constructed by methods known in the art. For example, mRNA (e.g., human) is isolated, cDNA is made and ligated into an expression vector (e.g., a bacteriophage derivative) such that it is capable of being expressed by the host cell into which it is then introduced. Various screening assays can then be used to select for the expressed DIAPH3 product. In one embodiment, anti-DIAPH3 antibodies can be used for selection.

In another embodiment of the invention, polymerase chain reaction (PCR) is used to amplify the desired sequence in a genomic or cDNA library, prior to selection. Oligonucleotide primers representing known DIAPH3-encoding sequences can be used as primers in PCR. In a preferred aspect, the oligonucleotide primers represent at least part of the conserved segments of strong homology between DIAPH3-encoding genes of different species, for example FH2 domains. The synthetic oligonucleotides may be utilized as primers to amplify by PCR sequences from RNA or DNA, preferably a cDNA library, of potential interest. Alternatively, one can synthesize degenerate primers for use in the PCR reactions.

In PCR according to the invention, the nucleic acid being amplified can include RNA or DNA, for example, mRNA, cDNA or genomic DNA from any eukaryotic species. PCR can be carried out, e.g., by use of a Perkin-Elmer Cetus thermal cycler and Taq polymerase. It is also possible to vary the stringency of hybridization conditions used in priming the PCR reactions, to allow for greater or lesser degrees of nucleotide sequence similarity between a known DIAPH3 nucleotide sequence and a nucleic acid homolog being isolated. For cross-species hybridization, low stringency conditions are preferred. For same-species hybridization, moderately stringent conditions are preferred. After successful amplification of a segment of a DIAPH3 homolog, that segment may be cloned, sequenced, and utilized as a probe to isolate a complete cDNA or genomic clone. This, in turn, will permit the determination of the gene's complete nucleotide sequence, the analysis of its expression, and the production of its protein product for functional analysis, as described infra. In this fashion, additional nucleotide sequences encoding DIAPH3 or DIAPH3 homologs may be identified.

The above recited methods are not meant to limit the following general description of methods by which clones of genes encoding DIAPH3 or homologs thereof may be obtained.

Any eukaryotic cell potentially can serve as the nucleic acid source for the molecular cloning of the DIAPH3 gene, DIAPH3 cDNA or a homolog thereof. The nucleic acid sequences encoding DIAPH3 can be isolated from vertebrate, mammalian, human, porcine, bovine, feline, avian, equine, canine, as well as additional primate sources. The DNA may be obtained by standard procedures known in the art from cloned DNA (e.g., a DNA “library”), by chemical synthesis, by cDNA cloning, or by the cloning of genomic DNA, or fragments thereof, purified from the desired cell, or by PCR amplification and cloning. (See, for example, Sambrook et al., MOLECULAR CLONING, A LABORATORY MANUAL, 2d. ed., Cold Spring Harbor Laboratory Press, Cold Spring Harbor, N.Y. (1989); Glover, D. M. (ed.), DNA CLONING: A PRACTICAL APPROACH, MRL Press, Ltd., Oxford, U.K. Vol. I, II (1985)). Clones derived from genomic DNA may contain regulatory and intron DNA regions in addition to coding regions; clones derived from cDNA will contain only exon sequences. Whatever the source, the gene should be cloned into a suitable vector for propagation of the gene.

In the cloning of the gene from genomic DNA, DNA fragments are generated, some of which will encode the desired gene. The DNA may be cleaved at specific sites using various restriction enzymes. Alternatively, one may use DNase in the presence of manganese to fragment the DNA, or the DNA can be physically sheared, as for example, by sonication. The linear DNA fragments can then be separated according to size by standard techniques, including but not limited to, agarose and polyacrylamide gel electrophoresis and column chromatography.

Once the DNA fragments are generated, identification of the specific DNA fragment containing the desired gene may be accomplished in a number of ways. For example, if a DIAPH3 gene (of any species) or its specific RNA, or a derivative thereof (see Section 5.6) is available and can be purified and labeled, the generated DNA fragments may be screened by nucleic acid hybridization to the labeled probe (Benton and Davis, Science 196:180 (1977); Grunstein and Hogness, Proc. Natl. Acad. Sci. U.S.A. 72:3961 (1975). Those DNA fragments with substantial homology to the probe will hybridize. It is also possible to identify the appropriate fragment by restriction enzyme digestion(s) and comparison of fragment sizes with those expected according to a known restriction map if such is available. Further selection can be carried out on the basis of the properties of the gene.

Alternatively, the presence of the gene may be detected by assays based on the physical, chemical, or immunological properties of its expressed product. For example, cDNA clones, or DNA clones that hybrid-select the proper mRNAs, can be selected that produce a protein having e.g., similar or identical electrophoretic migration, isoelectric focusing behavior, proteolytic digestion maps, effect on mitotic spindle pole formation, inhibition of cell proliferation activity, substrate binding activity, or antigenic properties as known for a specific DIAPH3. If an antibody to a particular DIAPH3 is available, that DIAPH3 may be identified by binding of labeled antibody to the clone(s) putatively producing the DIAPH3 in an ELISA (enzyme-linked immunosorbent assay)-type procedure.

A DIAPH3 or homolog thereof can also be identified by mRNA selection by nucleic acid hybridization followed by in vitro translation. In this procedure, fragments are used to isolate complementary mRNAs by hybridization. Such DNA fragments may represent available, purified DNA of another species containing a gene encoding DIAPH3. Immunoprecipitation analysis or functional assays (e.g., aggregation ability in vitro; binding to receptor; see infra) of the in vitro translation products of the isolated products of the isolated mRNAs identifies the mRNA and, therefore, the complementary DNA fragments that contain the desired sequences. In addition, specific mRNAs may be selected by adsorption of polysomes isolated from cells to immobilized antibodies specifically directed against a specific DIAPH3. A radiolabelled DIAPH3-encoding cDNA can be synthesized using the selected mRNA (from the adsorbed polysomes) as a template. The radiolabelled mRNA or cDNA may then be used as a probe to identify the DIAPH3-encoding DNA fragments from among other genomic DNA fragments.

Alternatives to isolating the DIAPH3 genomic DNA include, but are not limited to, chemically synthesizing the gene sequence itself from a known sequence or making cDNA to the mRNA which encodes DIAPH3. For example, RNA for the cloning of DIAPH3 cDNA can be isolated from cells that express a DIAPH3 gene. Other methods are possible and within the scope of the invention.

The identified and isolated DIAPH3- or DIAPH3 analog-encoding gene can then be inserted into an appropriate cloning vector. A large number of vector-host systems known in the art may be used. Possible vectors include, but are not limited to, plasmids or modified viruses, but the vector system must be compatible with the host cell used. Such vectors include, but are not limited to, bacteriophages such as lambda derivatives, or plasmids such as pBR322 or pUC plasmid derivatives or the pBluescript vector (Stratagene). The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved vector and DIAPH3-encoding gene or nucleic acid sequence may be modified by homopolymeric tailing. Recombinant molecules can be introduced into host cells via transformation, transfection, infection, electroporation, etc., so that many copies of the gene sequence are generated.

In an alternative method, the desired gene may be identified and isolated after insertion into a suitable cloning vector in a “shotgun” approach. Enrichment for the desired gene, for example, by size fractionization, can be done before insertion into the cloning vector.

In specific embodiments, transformation of host cells with recombinant DNA molecules that incorporate the isolated DIAPH3-encoding gene, cDNA, or synthesized DNA sequence enables generation of multiple copies of the gene. Thus, the gene may be obtained in large quantities by growing transformants, isolating the recombinant DNA molecules from the transformants and, when necessary, retrieving the inserted gene from the isolated recombinant DNA.

It will be understood that the RNA sequence equivalent of the nucleotide sequences provided herein can be easily and routinely generated by the substitution of thymine (T) residues with uracil (U) residues.

The DIAPH3-encoding or -related sequences provided by the instant invention include those nucleotide sequences encoding substantially the same amino acid sequences as found in native DIAPH3, and those encoded amino acid sequences with functionally equivalent amino acids, as well as those encoding other DIAPH3 derivatives, as described in Section 5.6 infra for derivatives of the DIAPH3 described herein.

The invention further relates to fragments and other derivatives of DIAPH3. Nucleic acids encoding such fragments or derivatives are thus also within the scope of the invention. The DIAPH3 gene, and DIAPH3-encoding nucleic acid sequences, of the invention include human and related genes (homologs) in other species. In specific embodiments, DIAPH3 and DIAPH3 are from vertebrates, or more particularly, mammals. In a preferred embodiment of the invention, DIAPH3 and DIAPH3 are of human origin. Production of the foregoing proteins and derivatives, e.g., by recombinant methods, is provided.

### 5.2 EXPRESSION OF GENES AND SEQUENCES ENCODING DIAPH3

The nucleotide sequence coding for DIAPH3 or a functionally active fragment or other derivative thereof (see Section 5.6), can be inserted into an appropriate expression vector, i.e., a vector which contains the necessary elements for the transcription and translation of the inserted protein-coding sequence. The necessary transcriptional and translational signals can also be supplied by the native DIAPH3 gene and/or its flanking regions. A variety of host-vector systems may be utilized to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used. In specific embodiments, the human DIAPH3 cDNA is expressed, or a sequence encoding a functionally active portion of human DIAPH3 encoded by the DIAPH3 gene is expressed. In yet another embodiment, a fragment of DIAPH3 comprising a domain of DIAPH3 is expressed.

Any of the methods previously described for the insertion of DNA fragments into a vector may be used to construct expression vectors containing a chimeric gene consisting of appropriate transcriptional/translational control signals and the protein coding sequences. These methods may include in vitro recombinant DNA and synthetic techniques and in vivo recombinants (genetic recombination). Expression of nucleic acid sequence encoding DIAPH3 or a peptide fragment thereof may be regulated by a second nucleic acid sequence so that DIAPH3 or a peptide fragment thereof is expressed in a host transformed with the recombinant DNA molecule. For example, expression of a DIAPH3 protein may be controlled by any promoter/enhancer element known in the art. In a specific embodiment, the promoter is heterologous to (i.e., not a native promoter of) the specific DIAPH3-encoding gene or nucleic acid sequence. Promoters that may be used to control expression of DIAPH3-encoding genes or nucleic acid sequences include, but are not limited to, the SV40 early promoter region (Bernoist and Chambon, Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc. Natl. Acad. Sci. U.S.A. 78:1441-1445 (1981)), the regulatory sequences of the metallothionein gene (Brinster et al., Nature 296:39-42 (1982)); prokaryotic expression vectors such as the β-lactamase promoter (Villa-Kamaroff et al., Proc. Natl. Acad. Sci. U.S.A. 75:3727-3731 (1978)), or the tat promoter (DeBoer et al., Proc. Natl. Acad. Sci. U.S.A. 80:21-25 (1983)); see also “Useful proteins from recombinant bacteria” in Scientific American, 242:74-94 (1980); plant expression vectors comprising the nopaline synthetase promoter region (Herrera-Estrella et al., Nature 303:209-213 (1983)) or the cauliflower mosaic virus 35S RNA promoter (Gardner et al., Nucleic Acids Res. 9:2871 (1981)), and the promoter of the photosynthetic enzyme ribulose biphosphate carboxylase (Herrera-Estrella et al., Nature 310:115-120 (1984)); promoter elements from yeast or other fungi such as the Gal4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region active in pancreatic acinar cells (Swift et al., Cell 38:639-646 (1984); Ornitz et al., Cold Spring Harbor Symp. Quant. Biol. 50:399-409 (1986); MacDonald, Hepatology 7:425-515 (1987)); insulin gene control region active in pancreatic beta cells (Hanahan, Nature 315:115-122 (1985)), immunoglobulin gene control region active in lymphoid cells (Grosschedl et al., Cell 38:647-658 (1984); Adames et al., Nature 318:533-538 (1985); Alexander et al., Mol. Cell. Biol. 7:1436-1444 (1987)), mouse mammary tumor virus control region active in testicular, breast, lymphoid and mast cells (Leder et al., Cell 45:485-495 (1986)), albumin gene control region active in liver (Pinkert et al., Genes and Devel. 1:268-276 (1987)), alpha-fetoprotein gene control region active in liver (Krumlauf et al., Mol. Cell. Biol. 5:1639-1648 (1985); Hammer et al., Science 235:53-58 (1987); alpha 1-antitrypsin gene control region active in the liver (Kelsey et al., Genes and Devel. 1 :161-171 (1987)), beta-globin gene control region active in myeloid cells (Mogram et al., Nature 315:338-340 (1985); Kollias et al., Cell 46:89-94 (1986); myelin basic protein gene control region active in oligodendrocyte cells in the brain (Readhead et al., Cell 48:703-712 (1987)); myosin light chain-2 gene control region active in skeletal muscle (Sani, Nature 314:283-286 (1985)), and gonadotropic releasing hormone gene control region active in the hypothalamus (Mason et al., Science 234:1372-1378 (1986)).

In a specific embodiment, a vector is used that comprises a promoter operably linked to a DIAPH3-encoding nucleic acid, one or more origins of replication, and, optionally, one or more selectable markers (e.g., an antibiotic resistance gene).

In a specific embodiment, an expression construct is made by subcloning the coding sequence from a DIAPH3-encoding gene or nucleic acid sequence into the EcoRI restriction site of each of the three pGEX vectors (Glutathione S-Transferase expression vectors; Smith and Johnson, Gene 7:31-40 (1988)). This allows for the expression of DIAPH3 from the subclone in the correct reading frame.

Expression vectors containing DIAPH3-encoding nucleic acid sequence inserts can be identified by three general approaches: (a) nucleic acid hybridization, (b) presence or absence of “marker” gene functions, and (c) expression of inserted sequences. In the first approach, the presence of a DIAPH3-encoding gene inserted in an expression vector can be detected by nucleic acid hybridization using probes comprising sequences that are homologous to an inserted DIAPH3-encoding gene. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a DIAPH3-encoding gene or nucleic acid sequence into the vector. For example, if the DIAPH3-encoding gene is inserted within the marker gene sequence of the vector, recombinants containing the insert can be identified by the absence of the marker gene function. In the third approach, recombinant expression vectors can be identified by assaying the specific DIAPH3 product expressed by the recombinant. Such assays can be based, for example, on the physical or functional properties of the DIAPH3 in in vitro assay systems, e.g., interaction with Rho GTPases, recruitment of actin subunits, or visible effects on mitotic spindle pole formation.

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors that can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered DIAPH3 may be controlled. Furthermore, different host cells have characteristic and specific mechanisms for the translational and post-translational processing and modification (e.g., glycosylation, phosphorylation of proteins. Appropriate cell lines or host systems can be chosen to ensure the desired modification and processing of the foreign protein expressed.

For example, expression in a bacterial system can be used to produce an unglycosylated core protein product. Expression in yeast will produce a glycosylated product. Expression in mammalian cells can be used to ensure “native” glycosylation of a heterologous protein. Furthermore, different vector/host expression systems may affect processing reactions to different degrees.

In other specific embodiments, DIAPH3, or fragment or derivative thereof, may be expressed as a fusion, or chimeric protein product, comprising the protein, fragment or derivative joined via a peptide bond to a protein sequence derived from a different protein. Such a chimeric product can be made by ligating the appropriate nucleic acid sequences encoding the desired amino acid sequences to each other by methods known in the art, in the proper coding frame, and expressing the chimeric product by methods commonly known in the art. In one embodiment, therefore, the invention includes an isolated nucleic acid comprising a sequence of at least 10 nucleotides encoding a chimeric DIAPH3, wherein the chimeric DIAPH3 displays at least one of the functional activities of the wild-type DIAPH3, and at least one non-DIAPH3 functional activity. Alternatively, such a chimeric product may be made by protein synthetic techniques, e.g., by use of a peptide synthesizer.

A person of skill in the art will appreciate that cDNA, genomic, and synthesized sequences can be cloned and expressed. One way to accomplish such expression is by transferring a DIAPH3 -encoding gene, DIAPH3 cDNA, or another nucleic acid encoding DIAPH3 or fragment thereof, to cells in tissue culture. The expression of the transferred nucleic acid may be controlled by its native promoter, or can be controlled by a non-native promoter. In addition to transferring a nucleic acid comprising a nucleic acid sequence encoding the entire DIAPH3 (i.e., equivalent to the wild type), the transferred nucleic acids can be any of the nucleic acids taught herein, e.g., nucleic acids that encode a functional portion of DIAPH3, or a protein having at least 60% sequence identity to the DIAPH3 disclosed herein, as compared over the length of DIAPH3, or a polypeptide having at least 60% sequence similarity to a DIAPH3 fragment, as compared over the length of the DIAPH3 fragment. Introduction of the nucleic acid into the cell is accomplished by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. The expressed DIAPH3 or fragments thereof are isolated and purified as described below.

### 5.3 Identification and Purification of DIAPH3 and Fragments Thereof

In particular aspects, the invention provides amino acid sequence of DIAPH3, preferably human DIAPH3, and fragments and derivatives thereof that comprise an antigenic determinant (i.e., a portion of a polypeptide that can be recognized by an antibody) or which are otherwise functionally active, as well as nucleic acid sequences encoding the foregoing. “Functionally active” DIAPH3 material as used herein refers to that material displaying one or more known functional activities associated with a full-length (wild-type) DIAPH3, e.g., activities associated with FH proteins; antigenicity (the ability to be bound by an antibody against DIAPH3, specifically, the ability to be bound by an antibody to a protein consisting of the amino acid sequence of SEQ ID NO: 3); immunogenicity (the ability to induce the production of an antibody that binds SEQ ID NO: 3), and so forth.

In one embodiment, the protein of the invention comprises less than the entire amino acid sequence of SEQ ID NO: 3. In other specific embodiments, the invention provides fragments of DIAPH3 consisting of at least 6, 10, 30, 50, 75, 100, 150, 200, 250, 300, 400, 450, 500, 600, 700, 800, 900, 1000, or 1100 amino acids that have less than the full-length DIAPH3 protein sequence. In another embodiment, said fragments of DIAPH3 consist of at least the C-terminal 6, 10, 30, 50, 75, 100, 150, 200, 250, 300, 400, 450, 500, 600, 700, 800, 900, 1000, or 1100 amino acids of SEQ ID NO: 3. In other embodiments, the proteins comprise or consist essentially of an FH2 domain of DIAPH3. For example, in one embodiment, the protein comprises amino acids 636-1152 of SEQ ID NO: 3; in another embodiment, the protein comprises amino acids 636-1110 of SEQ ID NO: 3. Fragments, or proteins comprising fragments, lacking the FH2 domain are also provided. Nucleic acids encoding the foregoing are also provided.

Once a recombinant that expresses the DIAPH3-encoding gene sequence, or part thereof, is identified, the resulting product can be analyzed. This analysis is achieved by assays based on the physical or functional properties of the product, including radioactive labeling of the product followed by analysis by gel electrophoresis, immunoassay, effects of the expressed product on motitic spindle pole formation in cells expressing the product, etc.

Once the DIAPH3, or analog, homolog or fragment thereof, is identified, it may be isolated and purified by standard methods including chromatography (e.g., ion exchange, affinity, and sizing column chromatography), centrifugation, differential solubility, or by any other standard technique for the purification of proteins. A DIAPH3 protein is “purified” when it is separated from at least half of the proteins associated with the cell that produces the DIAPH3 as measured by molecular weight or concentration in solution. In more specific embodiments, the DIAPH3 is purified to at least 80%, 90%, 95% or 99% purity; that is, the DIAPH3 protein comprises at least 80%, 90%, 95% or 99% by weight of the protein present. A solution comprising only DIAPH3 and a substantial amount of a carrier protein (such as albumin), for example, 10-20% carrier protein, with negligible amounts of other proteins, is considered purified. The functional properties of the purified DIAPH3 may be evaluated using any suitable assay (see Section 5.7).

Alternatively, once DIAPH3 produced by a recombinant is identified, the amino acid sequence of the protein can be deduced from the nucleotide sequence of the chimeric gene contained in the recombinant. As a result, the protein can be synthesized by standard chemical methods known in the art (e.g., see Hunkapiller et al., Nature 310:105-111 (1984)).

In another alternate embodiment, the native DIAPH3 protein can be purified from natural sources, by standard methods such as those described above (e.g., immunoaffinity purification).

In a specific embodiment of the present invention, DIAPH3, whether produced by recombinant DNA techniques or by chemical synthetic methods or by purification of native proteins, include but are not limited to those containing, as a primary amino acid sequence, all or part of the amino acid sequence substantially as depicted in FIGS. 1A-1E (SEQ ID NO: 3), as well as fragments and other derivatives thereof, including proteins homologous thereto.

### 5.4 Structure of DIAPH3 Genes and Homologs, and DIAPH3

The structure of the genes encoding DIAPH3, and the encoded DIAPH3, can be analyzed by various methods known in the art, as described in the following sections.

### 5.4.1 Genetic Analysis

The cloned DNA or cDNA corresponding to a DIAPH3-encoding gene can be analyzed by methods including, but not limited to, Southern hybridization (Southern, E. M., J. Mol. Biol. 98:503-517 (1975)), northern hybridization (see e.g., Freeman et al., Proc. Natl. Acad. Sci. U.S.A. 80:4094-4098 (1983)), restriction endonuclease mapping (Maniatis, T., MOLECULAR CLONING, A LABORATORY MANUAL, Cold Spring Harbor, N.Y. (1982)), and DNA sequence analysis. Polymerase chain reaction (PCR; U.S. Pat. Nos. 4,683,202, 4,683,195 and 4,889,818; Gyllenstein et al., Proc. Natl. Acad. Sci. U.S.A. 85:7652-7656 (1988); Ochman et al., Genetics 120:621-623 (1988); Loh et al., Science 243:217-220 (1989)) followed by Southern hybridization with a probe specific to a DIAPH3-encoding gene can allow the detection of that particular DIAPH3-encoding gene in DNA from various cell types from various vertebrate sources. Methods of amplification other than PCR are commonly known and can also be employed. In one embodiment, Southern hybridization can be used to determine the genetic linkage of a DIAPH3 gene. Northern hybridization analysis can be used to determine the expression of a DIAPH3 gene. Various cell types, at various states of development or activity can be tested for expression of a DIAPH3 gene. In one preferred embodiment, screening arrays comprising probes homologous to the exons of DIAPH3-encoding genes are used to determine the state of expression of these genes, or specific exons of these genes, in various cell types, under particular environmental or perturbance conditions, or in various vertebrates. The stringency of the hybridization conditions for both Southern and northern hybridization can be manipulated to ensure detection of nucleic acids with the desired degree of relatedness to the specific probe used. Modifications of these methods and other methods commonly known in the art can be used.

Restriction endonuclease mapping can be used to roughly determine the genetic structure of DIAPH3 or any other DIAPH3-encoding gene. Restriction maps derived by restriction endonuclease cleavage can be confirmed by DNA sequence analysis. The genetic structure of a DIAPH3-encoding gene can also be determined using scanning oligonucleotide arrays, wherein the expression of one exon is correlated with the expression of a plurality of neighboring exons, such that the correlation indicates the correlated exons are contained within the same gene. The structure so determined can be confirmed by PCR.

DNA sequence analysis can be performed by any techniques known in the art, including but not limited to the method of Maxam and Gilbert, Meth. Enzymol. 65:499-5601 (1980), the Sanger dideoxy method (Sanger, F., et al., Proc. Natl. Acad. Sci. U.S.A. 74:5463 (1977)), the use of T7 DNA polymerase (Tabor and Richardson, U.S. Pat. No. 4,795,699), or use of an automated DNA Sequenator (e.g., Applied Biosystems, Foster City, Calif.). The sequencing method may use radioactive or fluorescent labels.

### 5.4.2 Protein Analysis

The amino acid sequence of DIAPH3 or a homolog thereof can be derived by deduction from the DNA sequence, or alternatively, by direct sequencing of the protein, e.g., with an automated amino acid sequencer.

The protein sequence of DIAPH3 can be characterized by a hydrophilicity analysis (Hopp and Woods, Proc. Natl. Acad. Sci. U.S.A. 78:3824 (1981)). A hydrophilicity profile is used to identify the hydrophobic and hydrophilic regions of DIAPH3 or a homolog thereof and the corresponding regions of the gene sequence which encode such regions.

Secondary structural analysis (Chou and Fasman, Biochemistry 13:222 (1974)) can also be done, to identify regions of DIAPH3 or homologs thereof that assume specific secondary structures, such as α-helices, β-pleated sheets or turns.

Manipulation, translation, secondary structure prediction, open reading frame prediction and plotting, as well as determination of sequence homologies, can also be accomplished using computer software programs and nucleotide and protein sequence databases available in the art. Protein and/or nucleotide sequence homologies to known proteins or DNA sequences can be used to deduce the likely function of a DIAPH3, or domains thereof.

Other methods of structural analysis can also be employed. These include but are not limited to X-ray crystallography (Engstom, Biochem. Exp. Biol. 11:7-13 (1974)) and computer modeling (Fletterick, and Zoller, (eds.), “Computer Graphics and Molecular Modeling,” in CURRENT COMMUNICATIONS IN MOLECULAR BIOLOGY, Cold Spring Harbor Laboratory, Cold Spring Harbor, N.Y. (1986)).

### 5.5 Generation of Antibodies to DIAPH3 and Derivatives thereof

According to the invention, DIAPH3, its fragments, or other derivatives thereof may be used as an immunogen to generate antibodies which immunospecifically bind such an immunogen. Such antibodies include but are not limited to polyclonal, monoclonal, chimeric and single chain antibodies, as well as Fab fragments and an Fab expression library. In a specific embodiment, antibodies to human DIAPH3 are produced. In another specific embodiment, antibodies are produced that specifically bind to a protein the amino acid sequence of which consists of SEQ ID NO: 3. In another embodiment, antibodies to a domain of human DIAPH3 are produced. In a more specific embodiment, said antibody specifically binds the FH2 domain of a protein the amino acid sequence of which consists of SEQ ID NO: 3. In another specific embodiment, said antibody specifically binds to an epitope present within amino acids 1110-1152 of SEQ ID NO: 3. In another embodiment, antibodies to non-human DIAPH3 or a fragment thereof are produced. In a specific embodiment, fragments of DIAPH3, human or non-human, identified as containing hydrophilic regions are used as immunogens for antibody production. In a specific embodiment, a hydrophilicity analysis can be used to identify hydrophilic regions of DIAPH3, which are potential epitopes, and thus can be used as immunogens.

Various procedures known in the art may be used for the production of polyclonal antibodies to DIAPH3, or derivative thereof. In a particular embodiment, rabbit polyclonal antibodies to an epitope of DIAPH3 encoded by a sequence of SEQ ID NO: 1 or SEQ ID NO: 2 or a subsequence thereof, can be obtained. For the production of antibody, various host animals can be immunized by injection with native DIAPH3, or a synthetic version or derivative (e.g., fragment) thereof, including, but not limited to, rabbits, mice, rats, goats, bovines or horses. Various adjuvants may be used to increase the immunological response, depending on the host species. Adjuvants that may be used according to the present invention include, but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward a DIAPH3 sequence or derivative thereof, any technique that provides for the production of antibody molecules by continuous cell lines in culture may be used. For example, monoclonal antibodies may be prepared by the hybridoma technique originally developed by Kohler and Milstein, Nature 256:495-497 (1975), as well as the trioma technique, the human B-cell hybridoma technique (Kozbor et al., Immunol. Today 4:72 (1983)), or the EBV-hybridoma technique (Cole et al., in MONOCLONAL ANTIBODIES AND CANCER THERAPY, Alan R. Liss, Inc., pp. 77-96 (1985)). In an additional embodiment of the invention, monoclonal antibodies can be produced in germ-free animals utilizing recent technology (International Publication No. W08912690, published Dec. 28, 1989). According to the invention, human antibodies may be used and can be obtained by using human hybridomas (Cote et al., Proc. Natl. Acad. Sci. U.S.A., 80:2026-2030 (1983)) or by transforming human B cells with EBV virus in vitro (Cole et al., in MONOCLONAL ANTIBODIES AND CANCER THERAPY, Alan R. Liss, pp. 77-96 (1985)). Furthermore, according to the invention, techniques developed for the production of “chimeric antibodies” (Morrison et al., Proc. Natl. Acad. Sci. U.S.A. 81:6851-6855 (1984); Neuberger et al., Nature 312:604-608 (1984); Takeda et al., Nature 314:452-454 (1985)) can be used, wherein genes from a mouse antibody molecule specific to DIAPH3 are spliced to genes encoding a human antibody molecule of appropriate biological activity can be used; such antibodies are within the scope of this invention.

In addition, techniques have been developed for the production of humanized antibodies, and such humanized antibodies to DIAPH3 are within the scope of the present invention. (See, e.g., Queen, U.S. Pat. No. 5,585,089 and Winter, U.S. Pat. No. 5,225,539.) An immunoglobulin light or heavy chain variable region consists of a “framework” region interrupted by three hypervariable regions, referred to as complementarity determining regions (CDRs). The extent of the framework region and CDRs have been precisely defined (see, “Sequences of Proteins of Immunological Interest”, Kabat, E. et al., U.S. Department of Health and Human Services (1983)). Briefly, humanized antibodies are antibody molecules from non-human species having one or more CDRs from the non-human species and a framework region from a human immunoglobulin molecule.

According to the invention, techniques described for the production of single chain antibodies (U.S. Pat. No. 4,946,778) can be adapted to produce single chain antibodies specific to DIAPH3. An additional embodiment of the invention utilizes the techniques described for the construction of Fab expression libraries (Huse et al., DIAPH3 246:1275-1281 (1988)) to allow rapid and easy identification of monoclonal Fab fragments with the desired specificity for DIAPH3 or derivatives thereof. Antibody fragments that contain the idiotype of the molecule can be generated by known techniques. For example, such fragments include but are not limited to: the F(ab′), fragment which can be produced by pepsin digestion of the antibody molecule; the Fab′ fragments which can be generated by reducing the disulfide bridges of the F(ab′), fragment, the Fab fragments which can be generated by treating the antibody molecule with papain and a reducing agent, and Fv fragments.

In the production of antibodies, screening for the desired antibody can be accomplished by techniques known in the art, e.g. ELISA (enzyme-linked immunosorbent assay), RIA (radioimmunoassay) or RIBA (recombinant immunoblot assay). For example, to select antibodies which recognize a specific domain of DIAPH3, one may assay generated hybridomas for a product which binds to a DIAPH3 fragment containing such domain. For selection of an antibody that specifically binds a first DIAPH3 homolog but which does not specifically bind a second, different DIAPH3 homolog, one can select on the basis of positive binding to the first DIAPH3 homolog and a lack of binding to the second DIAPH3 homolog.

Antibodies specific to a domain of DIAPH3 or a homolog thereof are also provided. The foregoing antibodies can be used in methods known in the art relating to the localization and activity of the DIAPH3 of the invention, e.g., for imaging these proteins, measuring levels thereof in appropriate physiological samples, in diagnostic methods, etc.

In another embodiment of the invention, antibodies to DIAPH3 or homologs thereof, and antibody fragments thereof containing the binding domain are therapeutics (see infra). In a preferred embodiment, the antibodies are isolated or purified.

### 5.6 DIAPH3 AND DIAPH3 Derivatives

The invention further relates to DIAPH3 and derivatives thereof (including but not limited to fragments of DIAPH3). Nucleic acids encoding derivatives and fragments of DIAPH3 are also provided. In one embodiment, DIAPH3 is encoded by the DIAPH3-encoding nucleic acids described in Section 5.1 supra.

The production and use of derivatives produced through modification of DIAPH3-encoding genes, such as the DIAPH3 gene, DIAPH3 cDNA or the coding region of either thereof, are within the scope of the present invention. In a specific embodiment, the derivative is functionally active, i.e., capable of exhibiting one or more functional activities associated with a full-length, wild-type DIAPH3. As one example, such derivatives that have the desired immunogenicity or antigenicity can be used, for example, in immunoassays, for immunization, for inhibition of the activity of DIAPH3, etc. As another example, such derivatives that substantially have the desired DIAPH3 activity are provided. Derivatives that retain, or alternatively lack or inhibit, a desired DIAPH3 property of interest, a specific activity, such as activity associated with FH2 domains, can be used as inducers, or inhibitors, respectively, of such a property and its physiological correlates. A specific embodiment relates to a DIAPH3 fragment that can be bound by an antibody directed to the corresponding native DIAPH3. Derivatives of DIAPH3 can be tested for the desired activity(ies) by procedures known in the art, including but not limited to the assays described in Section 5.7.

In particular, derivatives of DIAPH3 can be made by altering the nucleotide sequences encoding them by substitutions, additions or deletions that provide for functionally equivalent protein molecules. In a specific embodiment, the alteration is made in a nucleic acid sequence encoding all or part of DIAPH3. Due to the degeneracy of nucleotide coding sequences, other DNA sequences that encode substantially the same amino acid sequence as a DIAPH3-encoding gene may be used in the practice of the present invention. These include but are not limited to nucleotide sequences comprising all or portions of DIAPH3-encoding genes that are altered by the substitution of different codons that encode the same amino acid residue within the sequence, thus producing a silent change.

Likewise, the DIAPH3 derivatives of the invention include, but are not limited to, those containing, as a primary amino acid sequence, all or part of the amino acid sequence of a DIAPH3 protein, including altered sequences in which functionally equivalent amino acid residues are substituted for residues within the sequence resulting in a silent or insubstantial change. For example, one or more amino acid residues within the sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

In specific embodiments, the invention provides DIAPH3 derivatives comprising 1, 2, 3, or up to 5, 10 or 20 amino acid substitutions as compared to SEQ ID NO: 3.

In a specific embodiment of the invention, proteins consisting of or comprising a fragment of DIAPH3 consisting of at least 30 (continuous) amino acids of DIAPH3 are provided. In other embodiments, the fragment consists of at least 40 or 50 amino acids of DIAPH3. In specific embodiments, such fragments are not larger than 35, 100 or 200 amino acids. Derivatives of DIAPH3 include but are not limited to those molecules comprising regions that are homologous to DIAPH3 or fragments thereof. In various embodiments, two amino acid sequences that are homologous share preferably at least 60% or 70%, more preferably at least 80% or 90%, and even more preferably at least 95% sequence identity over an amino acid sequence of identical size. When the alignment is done by a computer homology program known in the art, such as BLAST (blastp), the percent homology is calculated by dividing the number of amino acids in the DIAPH3 sequence or fragment thereof into the number of amino acids of the DIAPH3 sequence exactly matching the amino acid at the same position in the second sequence, where introduced gaps count as a mismatch, and where conservative changes count as a match. A BLAST comparison can also determine the “sequence similarity” between two proteins, where sequence similarity is defined as a positive score in, for example, a BLOSUM62 scoring matrix comparison of the two sequences.

Derivatives of DIAPH3 also include molecules whose encoding nucleic acid is capable of hybridizing to a DIAPH3-encoding sequence, under stringent, moderately stringent, or nonstringent conditions.

The DIAPH3 derivatives of the invention can be produced by various methods known in the art. The manipulations which result in their production can occur at the gene or protein level. For example, the cloned gene sequence of DIAPH3 or a homolog thereof can be modified by any of numerous strategies known in the art (Maniatis, MOLECULAR CLONING, A LABORATORY MANUAL, 2d. ed., Cold Spring Harbor Laboratory, Cold Spring Harbor, N.Y. (1990)). The sequence can be cleaved at appropriate sites with restriction endonuclease(s), followed by further enzymatic modification if desired, then isolated and ligated in vitro. In the production of a gene encoding a derivative of DIAPH3, care should be taken to ensure that the modified gene remains within the same translational reading frame as DIAPH3, uninterrupted by translational stop signals, in the gene region where the desired DIAPH3 activity is encoded.

Additionally, a DIAPH3-encoding nucleic acid sequence can be mutated in vitro or in vivo to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, chemical mutagenesis, in vitro site-directed mutagenesis (Hutchinson, et al., J. Biol. Chem. 253:6551(1978)), use of TAB linkers (Pharmacia), PCR using mutagenizing primers, and so forth.

Manipulations of a DIAPH3 sequence may also be made at the protein level. Included within the scope of the invention are DIAPH3 fragments or other derivatives that are differentially modified during or after translation, e.g., by glycosylation, acetylation, phosphorylation, amidation, derivatization by known protecting/blocking groups, proteolytic cleavage, or linkage to an antibody molecule or other cellular ligand. Any of numerous chemical modifications may be carried out by known techniques, including, but not limited to, specific chemical cleavage by cyanogen bromide, trypsin, chymotrypsin, papain, V8 protease, NaBH₄; acetylation, formylation, oxidation, reduction; metabolic synthesis in the presence of tunicamycin; and so forth.

In addition, derivatives of DIAPH3 can be chemically synthesized. For example, a peptide corresponding to a portion of DIAPH3 that comprises a desired domain, or which mediates the desired activity in vitro, can be synthesized by use of a peptide synthesizer. Furthermore, if desired, nonclassical amino acids or chemical amino acid analogs can be introduced as a substitution or addition into the particular DIAPH3 sequence. Non-classical amino acids include, but are not limited, to the D-isomers of the common amino acids, “-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general. Furthermore, the amino acid can be D (dextrorotary) or L (levorotary).

In a specific embodiment, the derivative of a DIAPH3 is a chimeric, or fusion, protein comprising a DIAPH3 protein or fragment thereof, preferably consisting of at least a domain or motif of DIAPH3, or at least 6 amino acids of DIAPH3, joined at its amino- or carboxy-terminus via a peptide bond to an amino acid sequence of a different protein. In one embodiment, such a chimeric protein is produced by recombinant expression of a nucleic acid encoding the protein, comprising a DIAPH3-coding sequence joined in-frame to a coding sequence for a different protein. Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g. by use of a peptide synthesizer. Chimeric genes comprising portions of a DIAPH3-encoding gene, fused to any heterologous protein-encoding sequences, may be constructed. A specific embodiment relates to a chimeric protein comprising a fragment of DIAPH3 of at least six amino acids.

Other specific embodiments of derivatives are described in the subsection below and examples sections infra.

In a specific embodiment, the invention relates to DIAPH3 derivatives; and fragments and derivatives of such fragments, that comprise, or alternatively consist of, one or more domains of DIAPH3, including but not limited to a functional (e.g., binding) fragment of DIAPH3.

In another specific embodiment, a molecule is provided that comprises one or more domains (or functional portion thereof) of DIAPH3 but that also lacks one or more domains (or functional portion thereof) of DIAPH3. In a particular examples, a DIAPH3 derivative is provided that lacks the FH2 domain. In another embodiment, a molecule is provided that comprises one or more domains (or functional portion thereof) of a DIAPH3 and that has one or more mutant (e.g., due to deletion or point mutation(s)) domains of DIAPH3 such that the mutant domain has increased or decreased function. In a specific embodiment, one, two, or three point mutations are present. A person of skill in the art would understand that fragments comprising one or more domains, or one or more mutant domains, may be derived from naturally-occurring variants of DIAPH3, or from DIAPH3 analogs of other species, as well.

### 5.7 Assays of DIAPH3 and DIAPH3 Derivatives

The functional activity of DIAPH3, and derivatives thereof, including, but not limited to, binding to profilin or to a Rho GTPase, and/or the mediation of Rho-directed actin fiber assembly, can be assayed by various methods. For example, in one embodiment, where one is assaying for the ability to bind or compete with the wild-type DIAPH3 for binding to an antibody raised against the protein, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

In another embodiment, in those situations where a DIAPH3-binding protein, such as a Rho-GTPase, is identified, the binding can be assayed, e.g., by means well-known in the art. In another embodiment, physiological correlates of the binding of DIAPH3 to its substrate(s) can be assayed.

### 5.8 DIAPH3 as a Diagnostic and Prognostic Marker in Breast Cancer

The human DIAPH3 gene was identified pursuant to a study in which over 25,000 separate and unique genetic markers were examined to identify those the expression of which in breast cancer tumor cells, when compared to the expression of the same markers in normal cells, could be used to differentiate patients having a good prognosis from those having a poor prognosis, where poor prognosis is defined as the occurrence of a distant breast cancer metastasis within five years of initial diagnosis. The expression of these markers in a cohort of 78 patients was analyzed, and a subset of 231 markers was collected which differentiated good prognosis from poor prognosis patients. Of these 231 markers, a preferred set of 70 markers, those whose expression was most strongly correlated or anti-correlated with the tumor condition, was established. The details of these experiments are disclosed in International Publication No. WO 02/103320, published Dec. 27, 2002, which is incorporated herein by reference in its entirety. The 231 markers are listed in Table 1. Table 2, below, lists the 70 preferred markers from Table 1. Each entry in Table 2 includes a GenBank Accession number or Contig number, the correlation or anticorrelation to the tumor condition, the sequence name where applicable, and a description of the sequence. Contig sequences were obtained from Phil Green EST contigs, which is a collection of EST contigs assembled by Dr. Phil Green et al at the University of Washington (Ewing and Green, Nat. Genet. 25(2):232-4 (2000)), available on the Internet at phrap.org/est_assembly/index.html.

TABLE 1
231 gene markers that distinguish patients with good
prognosis from patients with poor prognosis.

| GenBank | |
|---|---|
| Accession Number/ | |
| Contig Number | SEQ ID NO |
| AA555029_RC | SEQ ID NO 46 |
| AB020689 | SEQ ID NO 47 |
| AB032973 | SEQ ID NO 48 |
| AB033007 | SEQ ID NO 49 |
| AB033043 | SEQ ID NO 50 |
| AB037745 | SEQ ID NO 51 |
| AB037863 | SEQ ID NO 52 |
| AF052159 | SEQ ID NO 53 |
| AF052162 | SEQ ID NO 54 |
| AF055033 | SEQ ID NO 55 |
| AF073519 | SEQ ID NO 56 |
| AF148505 | SEQ ID NO 57 |
| AF155117 | SEQ ID NO 58 |
| AF161553 | SEQ ID NO 59 |
| AF201951 | SEQ ID NO 60 |
| AF257175 | SEQ ID NO 61 |
| AJ224741 | SEQ ID NO 62 |
| AK000745 | SEQ ID NO 63 |
| AL050021 | SEQ ID NO 64 |
| AL050090 | SEQ ID NO 65 |
| AL080059 | SEQ ID NO 66 |
| AL080079 | SEQ ID NO 67 |
| AL080110 | SEQ ID NO 68 |
| AL133603 | SEQ ID NO 69 |
| AL133619 | SEQ ID NO 70 |
| AL137295 | SEQ ID NO 71 |
| AL137502 | SEQ ID NO 72 |
| AL137514 | SEQ ID NO 73 |
| AL137718 | SEQ ID NO 4 |
| AL355708 | SEQ ID NO 74 |
| D25328 | SEQ ID NO 75 |
| L27560 | SEQ ID NO 76 |
| M21551 | SEQ ID NO 77 |
| NM_000017 | SEQ ID NO 78 |
| NM_000096 | SEQ ID NO 79 |
| NM_000127 | SEQ ID NO 80 |
| NM_000158 | SEQ ID NO 81 |
| NM_000224 | SEQ ID NO 82 |
| NM_000286 | SEQ ID NO 83 |
| NM_000291 | SEQ ID NO 84 |
| NM_000320 | SEQ ID NO 85 |
| NM_000436 | SEQ ID NO 86 |
| NM_000507 | SEQ ID NO 87 |
| NM_000599 | SEQ ID NO 88 |
| NM_000788 | SEQ ID NO 89 |
| NM_000849 | SEQ ID NO 90 |
| NM_001007 | SEQ ID NO 91 |
| NM_001124 | SEQ ID NO 92 |
| NM_001168 | SEQ ID NO 93 |
| NM_001216 | SEQ ID NO 94 |
| NM_001280 | SEQ ID NO 95 |
| NM_001282 | SEQ ID NO 96 |
| NM_001333 | SEQ ID NO 97 |
| NM_001673 | SEQ ID NO 98 |
| NM_001809 | SEQ ID NO 99 |
| NM_001827 | SEQ ID NO 100 |
| NM_001905 | SEQ ID NO 101 |
| NM_002019 | SEQ ID NO 102 |
| NM_002073 | SEQ ID NO 103 |
| NM_002358 | SEQ ID NO 104 |
| NM_002570 | SEQ ID NO 105 |
| NM_002808 | SEQ ID NO 106 |
| NM_002811 | SEQ ID NO 107 |
| NM_002900 | SEQ ID NO 108 |
| NM_002916 | SEQ ID NO 109 |
| NM_003158 | SEQ ID NO 110 |
| NM_003234 | SEQ ID NO 111 |
| NM_003239 | SEQ ID NO 112 |
| NM_003258 | SEQ ID NO 113 |
| NM_003376 | SEQ ID NO 114 |
| NM_003600 | SEQ ID NO 115 |
| NM_003607 | SEQ ID NO 116 |
| NM_003662 | SEQ ID NO 117 |
| NM_003676 | SEQ ID NO 118 |
| NM_003748 | SEQ ID NO 119 |
| NM_003862 | SEQ ID NO 120 |
| NM_003875 | SEQ ID NO 121 |
| NM_003878 | SEQ ID NO 122 |
| NM_003882 | SEQ ID NO 123 |
| NM_003981 | SEQ ID NO 124 |
| NM_004052 | SEQ ID NO 125 |
| NM_004163 | SEQ ID NO 126 |
| NM_004336 | SEQ ID NO 127 |
| NM_004358 | SEQ ID NO 128 |
| NM_004456 | SEQ ID NO 129 |
| NM_004480 | SEQ ID NO 130 |
| NM_004504 | SEQ ID NO 131 |
| NM_004603 | SEQ ID NO 132 |
| NM_004701 | SEQ ID NO 133 |
| NM_004702 | SEQ ID NO 134 |
| NM_004798 | SEQ ID NO 135 |
| NM_004911 | SEQ ID NO 136 |
| NM_004994 | SEQ ID NO 137 |
| NM_005196 | SEQ ID NO 138 |
| NM_005342 | SEQ ID NO 139 |
| NM_005496 | SEQ ID NO 140 |
| NM_005563 | SEQ ID NO 141 |
| NM_005915 | SEQ ID NO 142 |
| NM_006096 | SEQ ID NO 143 |
| NM_006101 | SEQ ID NO 144 |
| NM_006115 | SEQ ID NO 145 |
| NM_006117 | SEQ ID NO 146 |
| NM_006201 | SEQ ID NO 147 |
| NM_006265 | SEQ ID NO 148 |
| NM_006281 | SEQ ID NO 149 |
| NM_006372 | SEQ ID NO 150 |
| NM_006681 | SEQ ID NO 151 |
| NM_006763 | SEQ ID NO 152 |
| NM_006931 | SEQ ID NO 153 |
| NM_007036 | SEQ ID NO 154 |
| NM_007203 | SEQ ID NO 155 |
| NM_012177 | SEQ ID NO 156 |
| NM_012214 | SEQ ID NO 157 |
| NM_012261 | SEQ ID NO 158 |
| NM_012429 | SEQ ID NO 159 |
| NM_013262 | SEQ ID NO 160 |
| NM_013296 | SEQ ID NO 161 |
| NM_013437 | SEQ ID NO 162 |
| NM_014078 | SEQ ID NO 163 |
| NM_014109 | SEQ ID NO 164 |
| NM_014321 | SEQ ID NO 165 |
| NM_014363 | SEQ ID NO 166 |
| NM_014750 | SEQ ID NO 167 |
| NM_014754 | SEQ ID NO 168 |
| NM_014791 | SEQ ID NO 169 |
| NM_014875 | SEQ ID NO 170 |
| NM_014889 | SEQ ID NO 171 |
| NM_014968 | SEQ ID NO 172 |
| NM_015416 | SEQ ID NO 173 |
| NM_015417 | SEQ ID NO 174 |
| NM_015434 | SEQ ID NO 175 |
| NM_015984 | SEQ ID NO 176 |
| NM_016337 | SEQ ID NO 177 |
| NM_016359 | SEQ ID NO 178 |
| NM_016448 | SEQ ID NO 179 |
| NM_016569 | SEQ ID NO 180 |
| NM_016577 | SEQ ID NO 181 |
| NM_017779 | SEQ ID NO 182 |
| NM_018004 | SEQ ID NO 183 |
| NM_018098 | SEQ ID NO 184 |
| NM_018104 | SEQ ID NO 185 |
| NM_018120 | SEQ ID NO 186 |
| NM_018136 | SEQ ID NO 187 |
| NM_018265 | SEQ ID NO 188 |
| NM_018354 | SEQ ID NO 189 |
| NM_018401 | SEQ ID NO 190 |
| NM_018410 | SEQ ID NO 191 |
| NM_018454 | SEQ ID NO 192 |
| NM_018455 | SEQ ID NO 193 |
| NM_019013 | SEQ ID NO 194 |
| NM_020166 | SEQ ID NO 195 |
| NM_020188 | SEQ ID NO 196 |
| NM_020244 | SEQ ID NO 197 |
| NM_020386 | SEQ ID NO 198 |
| NM_020675 | SEQ ID NO 199 |
| NM_020974 | SEQ ID NO 200 |
| R70506_RC | SEQ ID NO 201 |
| U45975 | SEQ ID NO 202 |
| U58033 | SEQ ID NO 203 |
| U82987 | SEQ ID NO 204 |
| U96131 | SEQ ID NO 205 |
| X05610 | SEQ ID NO 206 |
| X94232 | SEQ ID NO 207 |
| Contig753_RC | SEQ ID NO 208 |
| Contig1778_RC | SEQ ID NO 209 |
| Contig2399_RC | SEQ ID NO 210 |
| Contig2504_RC | SEQ ID NO 211 |
| Contig3902_RC | SEQ ID NO 212 |
| Contig4595 | SEQ ID NO 213 |
| Contig8581_RC | SEQ ID NO 214 |
| Contig13480_RC | SEQ ID NO 215 |
| Contig17359_RC | SEQ ID NO 216 |
| Contig20217_RC | SEQ ID NO 217 |
| Contig21812_RC | SEQ ID NO 218 |
| Contig24252_RC | SEQ ID NO 219 |
| Contig25055_RC | SEQ ID NO 220 |
| Contig25343_RC | SEQ ID NO 221 |
| Contig25991 | SEQ ID NO 222 |
| Contig27312_RC | SEQ ID NO 223 |
| Contig28552_RC | SEQ ID NO 5 |
| Contig32125_RC | SEQ ID NO 224 |
| Contig32185_RC | SEQ ID NO 225 |
| Contig33814_RC | SEQ ID NO 226 |
| Contig34634_RC | SEQ ID NO 227 |
| Contig35251_RC | SEQ ID NO 228 |
| Contig37063_RC | SEQ ID NO 229 |
| Contig37598 | SEQ ID NO 230 |
| Contig38288_RC | SEQ ID NO 231 |
| Contig40128_RC | SEQ ID NO 232 |
| Contig40831_RC | SEQ ID NO 233 |
| Contig41413_RC | SEQ ID NO 234 |
| Contig41887_RC | SEQ ID NO 235 |
| Contig42421_RC | SEQ ID NO 236 |
| Contig43747_RC | SEQ ID NO 237 |
| Contig44064_RC | SEQ ID NO 238 |
| Contig44289_RC | SEQ ID NO 239 |
| Contig44799_RC | SEQ ID NO 240 |
| Contig45347_RC | SEQ ID NO 241 |
| Contig45816_RC | SEQ ID NO 242 |
| Contig46218_RC | SEQ ID NO 6 |
| Contig46223_RC | SEQ ID NO 243 |
| Contig46653_RC | SEQ ID NO 244 |
| Contig46802_RC | SEQ ID NO 245 |
| Contig47405_RC | SEQ ID NO 246 |
| Contig48328_RC | SEQ ID NO 247 |
| Contig49670_RC | SEQ ID NO 248 |
| Contig50106_RC | SEQ ID NO 249 |
| Contig50410 | SEQ ID NO 250 |
| Contig50802_RC | SEQ ID NO 251 |
| Contig51464_RC | SEQ ID NO 252 |
| Contig51519_RC | SEQ ID NO 253 |
| Contig51749_RC | SEQ ID NO 254 |
| Contig51963 | SEQ ID NO 255 |
| Contig53226_RC | SEQ ID NO 256 |
| Contig53268_RC | SEQ ID NO 257 |
| Contig53646_RC | SEQ ID NO 258 |
| Contig53742_RC | SEQ ID NO 259 |
| Contig55188_RC | SEQ ID NO 260 |
| Contig55313_RC | SEQ ID NO 261 |
| Contig55377_RC | SEQ ID NO 262 |
| Contig55725_RC | SEQ ID NO 263 |
| Contig55813_RC | SEQ ID NO 264 |
| Contig55829_RC | SEQ ID NO 265 |
| Contig56457_RC | SEQ ID NO 266 |
| Contig57595 | SEQ ID NO 267 |
| Contig57864_RC | SEQ ID NO 268 |
| Contig58368_RC | SEQ ID NO 269 |
| Contig60864_RC | SEQ ID NO 270 |
| Contig63102_RC | SEQ ID NO 271 |
| Contig63649_RC | SEQ ID NO 272 |
| Contig64688 | SEQ ID NO 273 |

TABLE 2
70 Preferred prognosis markers drawn from Table 1.

| GenBank | | | |
|---|---|---|---|
| Accession Number/ | | | |
| Contig Number | Correlation | Sequence Name | Description |
| AL080059 | −0.527150 | | Homo sapiens mRNA for |
| | | | KIAA1750 protein, partial cds |
| Contig63649_RC | −0.468130 | | ESTs |
| Contig46218_RC | −0.432540 | | ESTs |
| NM_016359 | −0.424930 | LOC51203 | clone HQ0310 PRO0310p1 |
| AA555029_RC | −0.424120 | | ESTs |
| NM_003748 | 0.420671 | ALDH4 | aldehyde dehydrogenase 4 |
| | | | (glutamate gamma-semialdehyde |
| | | | dehydrogenase; pyrroline-5- |
| | | | carboxylate dehydrogenase) |
| Contig38288_RC | −0.414970 | | ESTs, Weakly similar to ISHUSS |
| | | | protein disulfide-isomerase |
| | | | [H. sapiens] |
| NM_003862 | 0.410964 | FGF18 | fibroblast growth factor 18 |
| Contig28552_RC | −0.409260 | | Homo sapiens mRNA; cDNA |
| | | | DKFZp434C0931 (from clone |
| | | | DKFZp434C0931); partial cds |
| Contig32125_RC | 0.409054 | | ESTs |
| U82987 | 0.407002 | BBC3 | Bcl-2 binding component 3 |
| AL137718 | −0.404980 | | Homo sapiens mRNA; cDNA |
| | | | DKFZp434C0931 (from clone |
| | | | DKFZp434C0931); partial cds |
| AB037863 | 0.402335 | KIAA1442 | KIAA1442 protein |
| NM_020188 | −0.400070 | DC13 | DC13 protein |
| NM_020974 | 0.399987 | CEGP1 | CEGP1 protein |
| NM_000127 | −0.399520 | EXT1 | exostoses (multiple) 1 |
| NM_002019 | −0.398070 | FLT1 | fms-related tyrosine kinase 1 |
| | | | (vascular endothelial growth |
| | | | factor/vascular permeability factor |
| | | | receptor) |
| NM_002073 | −0.395460 | GNAZ | guanine nucleotide binding protein |
| | | | (G protein), alpha z polypeptide |
| NM_000436 | −0.392120 | OXCT | 3-oxoacid CoA transferase |
| NM_004994 | −0.391690 | MMP9 | matrix metalloproteinase 9 |
| | | | (gelatinase B, 92 kD gelatinase, |
| | | | 92 kD type IV collagenase) |
| Contig55377_RC | 0.390600 | | ESTs |
| Contig35251_RC | −0.390410 | | Homo sapiens cDNA: FLJ22719 |
| | | | fis, clone HSI14307 |
| Contig25991 | −0.390370 | ECT2 | epithelial cell transforming |
| | | | sequence 2 oncogene |
| NM_003875 | −0.386520 | GMPS | guanine monphosphate synthetase |
| NM_006101 | −0.385890 | HEC | highly expressed in cancer, rich in |
| | | | leucine heptad repeats |
| NM_003882 | 0.384479 | WISP1 | WNT1 inducible signaling |
| | | | pathway protein 1 |
| NM_003607 | −0.384390 | PK428 | Ser-Thr protein kinase related to |
| | | | the myotonic dystrophy protein |
| | | | kinase |
| AF073519 | −0.383340 | SERF1A | small EDRK-rich factor 1A |
| | | | (telomeric) |
| AF052162 | −0.380830 | FLJ12443 | hypothetical protein FLJ12443 |
| NM_000849 | 0.380831 | GSTM3 | glutathione S-transferase M3 |
| | | | (brain) |
| Contig32185_RC | −0.379170 | | Homo sapiens cDNA FLJ13997 |
| | | | fis, clone Y79AA1002220 |
| NM_016577 | −0.376230 | RAB6B | RAB6B, member RAS oncogene |
| | | | family |
| Contig48328_RC | 0.375252 | | ESTs, Weakly similar to T17248 |
| | | | hypothetical protein |
| | | | DKFZp586G1122.1 [H. sapiens] |
| Contig46223_RC | 0.374289 | | ESTs |
| NM_015984 | −0.373880 | UCH37 | ubiquitin C-terminal hydrolase |
| | | | UCH37 |
| NM_006117 | 0.373290 | PECI | peroxisomal D3,D2-enoyl-CoA |
| | | | isomerase |
| AK000745 | −0.373060 | | Homo sapiens cDNA FLJ20738 |
| | | | fis, clone HEP08257 |
| Contig40831_RC | −0.372930 | | ESTs |
| NM_003239 | 0.371524 | TGFB3 | transforming growth factor, beta 3 |
| NM_014791 | −0.370860 | KIAA0175 | KIAA0175 gene product |
| X05610 | −0.370860 | COL4A2 | collagen, type IV, alpha 2 |
| NM_016448 | −0.369420 | L2DTL | L2DTL protein |
| NM_018401 | 0.368349 | HSA250839 | gene for serine/threonine protein |
| | | | kinase |
| NM_000788 | −0.367700 | DCK | deoxycytidine kinase |
| Contig51464_RC | −0.367450 | FLJ22477 | hypothetical protein FLJ22477 |
| AL080079 | −0.367390 | DKFZP564D0462 | hypothetical protein |
| | | | DKFZp564D0462 |
| NM_006931 | −0.366490 | SLC2A3 | solute carrier family 2 (facilitated |
| | | | glucose transporter), member 3 |
| AF257175 | 0.365900 | | Homo sapiens hepatocellular |
| | | | carcinoma-associated antigen 64 |
| | | | (HCA64) mRNA, complete cds |
| NM_014321 | −0.365810 | ORC6L | origin recognition complex, |
| | | | subunit 6 (yeast homolog)-like |
| NM_002916 | −0.365590 | RFC4 | replication factor C (activator 1) 4 |
| | | | (37 kD) |
| Contig55725_RC | −0.365350 | | ESTs, Moderately similar to |
| | | | T50635 hypothetical protein |
| | | | DKFZp762L0311.1 [H. sapiens] |
| Contig24252_RC | −0.364990 | | ESTs |
| AF201951 | 0.363953 | CFFM4 | high affinity immunoglobulin |
| | | | epsilon receptor beta subunit |
| NM_005915 | −0.363850 | MCM6 | minichromosome maintenance |
| | | | deficient (mis5, S. pombe) 6 |
| NM_001282 | 0.363326 | AP2B1 | adaptor-related protein complex 2, |
| | | | beta 1 subunit |
| Contig56457_RC | −0.361650 | TMEFF1 | transmembrane protein with EGF- |
| | | | like and two follistatin-like |
| | | | domains 1 |
| NM_000599 | −0.361290 | IGFBP5 | insulin-like growth factor binding |
| | | | protein 5 |
| NM_020386 | −0.360780 | LOC57110 | H-REV107 protein-related protein |
| NM_014889 | −0.360040 | MP1 | metalloprotease 1 (pitrilysin |
| | | | family) |
| AF055033 | −0.359940 | IGFBP5 | insulin-like growth factor binding |
| | | | protein 5 |
| NM_006681 | −0.359700 | NMU | neuromedin U |
| NM_007203 | −0.359570 | AKAP2 | A kinase (PRKA) anchor protein 2 |
| Contig63102_RC | 0.359255 | FLJ11354 | hypothetical protein FLJ11354 |
| NM_003981 | −0.358260 | PRC1 | protein regulator of cytokinesis 1 |
| Contig20217_RC | −0.357880 | | ESTs |
| NM_001809 | −0.357720 | CENPA | centromere protein A (17 kD) |
| Contig2399_RC | −0.356600 | SM-20 | similar to rat smooth muscle |
| | | | protein SM-20 |
| NM_004702 | −0.356600 | CCNE2 | cyclin E2 |
| NM_007036 | −0.356540 | ESM1 | endothelial cell-specific molecule 1 |
| NM_018354 | −0.356000 | FLJ11190 | hypothetical protein FLJ11190 |

Three of the most strongly correlated markers, AL137718 (SEQ ID NO: 4), Contig28552 (SEQ ID NO: 5) and Contig46218 (SEQ ID NO: 6) were markers whose upregulation, in comparison to their expression in nontumor cells, correlated with a poor prognosis. A BLAT search of one of the markers, AL137718, revealed a predicted gene that overlapped a second marker, Contig28552. Using these sequences, and the sequence of Contig46218, to design appropriate RT-PCR and sequencing primers (see Example 1), the full-length DIAPH3 cDNA was sequenced and elucidated.

Because the DIAPH3 cDNA sequence was identified using the sequences of three markers whose expression is strongly correlated with the presence of breast cancer and a poor prognosis, the overexpression of DIAPH3, compared to expression in normal cells, will also correlate strongly with a poor prognosis. DIAPH3 is therefore a useful breast cancer diagnostic and prognostic marker.

Thus, in one embodiment, the invention provides a method of diagnosing an individual as having breast cancer comprising comparing the level of expression of a nucleic acid encoding SEQ ID NO: 3 in a breast cell sample from said individual to a control level of expression of said nucleic acid encoding SEQ ID NO: 3; and classifying said individual as having breast cancer if said level of expression of said nucleic acid in a breast cell sample from said individual is greater than said control level of expression. In a specific embodiment, said patient is classified as having breast cancer if the logarithm of the ratio of said level of expression of a nucleic acid encoding SEQ ID NO: 3 in a breast cell sample from said individual to said control level of expression is 0.3 or greater. In these, and other, embodiments, a control level of expression may be, for example, the level of expression of a nucleic acid encoding SEQ ID NO: 3 in a breast cell sample from an individual known not to have breast cancer, or a standard level of expression known for non-malignant breast cell samples in a species or population. In a specific embodiment, said level of expression of a nucleic acid encoding SEQ ID NO: 3 in a sample derived from breast cells is determined by hybridizing said nucleic acid with an oligonucleotide complementary and hybridizable to nucleotides 1-2384 or nucleotides 2927-4331 of SEQ ID NO: 1, and determining the amount of said hybridization. In another specific embodiment, said level of expression of a nucleic acid encoding SEQ ID NO: 3 in a sample derived from breast cells is determined by hybridizing said nucleic acid with an oligonucleotide complementary and hybridizable to nucleotides 1-862, 2927-3045, or 3412-3929 of SEQ ID NO: 1, and determining the amount of said hybridization.

In another embodiment of the invention, the prognosis of a breast cancer patient may be predicted by a method comprising: (a) determining the level of expression of a nucleic acid encoding SEQ ID NO: 3 in a sample derived from breast cancer tumor cells from said patient; (b) comparing the level of expression in said sample to a control level of expression; and (c) predicting that the patient will have a poor prognosis if said level of expression in the tumor sample is higher than the level of expression in the control. In a more specific embodiment, said level of expression of a nucleic acid encoding SEQ ID NO: 3 in said sample is higher than the level of expression in said control. In a preferred embodiment, the level in said sample is significantly higher than the level in said control. In a preferred embodiment, a first level is “significantly higher” than a second level when the log ratio of the first level to the second level is at least 0.3. In a more specific embodiment of the above method, said determining is accomplished by hybridizing said nucleic acids in a sample to an oligonucleotide, wherein said oligonucleotide hybridizable to SEQ ID NO: 1 or its complement; and determining the amount of hybridized oligonucleotide. In a more specific embodiment, the sequence of said oligonucleotide is not found in AL137718, Contig28552 or Contig46218; and determining the amount of hybridized oligonucleotide. In another more specific embodiment, said level of expression of a nucleic acid encoding SEQ ID NO: 3 in a sample derived from breast cells is determined by hybridizing said nucleic acid with an oligonucleotide complementary and hybridizable to nucleotides 1-2384 or nucleotides 2927-4331 of SEQ ID NO: 1, and determining the amount of said hybridization, wherein said amount of hybridization indicates said level of expression. In another more specific embodiment, said level of expression of a nucleic acid encoding SEQ ID NO: 3 in a sample derived from breast cells is determined by hybridizing said nucleic acid with an oligonucleotide complementary and hybridizable to nucleotides 1-862, 2927-3045, or 3412-3929 of SEQ ID NO: 1, and determining the amount of said hybridization, wherein said amount of hybridization indicates said level of expression. In another specific embodiment, said oligonucleotide is a probe on a microarray. In a more specific example, said oligonucleotide is one of a plurality of probes on a microarray, wherein said plurality comprises probes complementary and hybridizable to nucleic acids encoded by five different breast cancer-related markers that do not encode SEQ ID NO: 3. In another specific embodiment, said oligonucleotide is one of a plurality of probes on a microarray, wherein said plurality comprises probes complementary and hybridizable to nucleic acids encoded by twenty different breast cancer-related markers that do not encode SEQ ID NO: 3. Such markers may be any marker identified as being related to or indicative of the presence of breast cancer. Preferably, said 5 or 20 different breast cancer-related markers are selected from the markers disclosed in International Publication No. WO 02/103320, published Dec. 27, 2002, entitled “Diagnosis and Prognosis of Breast Cancer Patients,” which is incorporated by reference herein in its entirety. For example, in one preferred embodiment, said five or twenty different breast cancer-related markers are present in Table 1. In another preferred embodiment, said five or twenty different breast cancer-related markers are present in Table 2. In another preferred embodiment, said 20 different breast cancer-related markers have the following GenBank Accession Numbers or Contig Numbers: AL080059; Contig63649_RC; Contig46218_RC; NM_{—}016359; AA555029_RC; NM_{—}003748; Contig38288_RC; NM_{—}003862; Contig28552_RC; Contig32125_RC; U82987; AL137718; AB037863; KIAA1442; NM_{—}020188; NM_{—}020974; NM_{—}000127; NM_{—}002019; NM_{—}002073; and NM_{—}000436. Contig sequences were obtained from Phil Green EST contigs, which is a collection of EST contigs assembled by Dr. Phil Green et al at the University of Washington (Ewing and Green, Nat. Genet. 25(2):232-4 (2000)), available on the Internet at phrap.org/est_assembly/index.html. “Breast cancer-related” means that the expression of the marker in breast cancer tumor cells is correlated with the breast cancer state and is significantly different than the marker's expression in normal cells.

Levels of DIAPH3 protein, alone or in combination with other proteins encoded by breast cancer-related marker genes, may also be determined in order to diagnose, or to predict the prognosis of, a breast cancer patient. For example, monitoring of levels of proteins encoded by breast cancer-related marker genes can be carried out by constructing a microarray in which binding sites comprise immobilized, preferably monoclonal, antibodies specific to a plurality of protein species encoded by the marker genes. Preferably, antibodies are present for a substantial fraction of the proteins encoded by the breast cancer-related marker genes. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes). In a preferred embodiment, monoclonal antibodies are raised against synthetic peptide fragments designed based on genomic sequence of the cell. With such an antibody array, proteins from the cell are contacted to the array and their binding is assayed with assays known in the art.

Thus, in one embodiment, the invention provides a method of diagnosing an individual as having breast cancer comprising comparing the level of a protein the amino acid sequence of which consists of SEQ ID NO: 3 in a sample derived from breast cells of said individual to a control level of said protein; and classifying said individual as having breast cancer if said level of protein in said sample from said individual is higher than said control level of said protein. In a more specific embodiment, said individual is classified as having breast cancer if said level of level of a protein the amino acid sequence of which consists of SEQ ID NO: 3 in a sample derived from breast cells of said individual is higher than said control level of said protein. In another embodiment of the invention, the prognosis of a breast cancer patient may be predicted by determining the level of a protein comprising SEQ ID NO: 3 in sample derived from breast cancer tumor cells of said patient; comparing the level of said protein in said sample to a control level of said protein; and predicting that the patient will have a poor prognosis if said level of said protein in said sample is significantly higher than is significantly higher than said control level of said protein. In a specific embodiment, said determining is carried out by a method comprising: (a) contacting said protein comprising SEQ ID NO: 3 from said sample derived from breast cancer tumor cells with an antibody that specifically binds said protein; and (b) determining the amount of antibody bound to said protein, wherein said amount of antibody bound to said protein indicates said level of said protein in said breast cancer tumor sample. In these, and other, embodiments, a control may be, for example, the level of DIAPH3 in a breast cell sample from an individual known not to have breast cancer.

It should be noted that, in the present invention, the expression of the DIAPH3 gene (i.e., the gene encoding SEQ ID NO: 3) may not be the sole indicator used in the diagnosis or prognosis of breast cancer. The expression of one of the nucleotide or amino acid sequences of the invention may be used in conjunction with, and correlated to, any other biochemical or clinical indicator of the presence, absence, or prognosis of a breast cancer. Thus, the terms “diagnosis” and “prognosis,” as used herein, encompass the use of the nucleotide or amino acid sequences described herein in screening for breast cancer, in determining the likelihood of the presence of breast cancer, and in supporting a diagnosis or prognosis of breast cancer in combination with other indicators of breast cancer.

The invention also provides kits for the facilitation of the diagnostic and/or prognostic methods of the invention. Thus, in one embodiment, the invention provides a kit for the diagnosis and/or prognosis of breast cancer, comprising in a container an oligonucleotide that hybridizes to the DIAPH3 coding sequence (i.e., SEQ ID NO: 2) under stringent conditions, wherein said oligonucleotide is at least 12 nucleotides in length and wherein the sequence of said oligonucleotide is not wholly present in Contig28552, Contig46218, or AL137718. In another embodiment, the invention provides a kit comprising in a container an oligonucleotide that hybridizes to SEQ ID NO: 1 under stringent conditions, wherein said oligonucleotide is at least 12 nucleotides in length, and is complementary and hybridizable to nucleotides 1-862, 2927-3045, or 3412-3929 of SEQ ID NO: 1. In a more specific embodiment, said oligonucleotide is a probe on a microarray. In an even more specific embodiment, said microarray comprises at least five breast cancer-related markers other than a nucleotide sequence that encodes SEQ ID NO: 3. In another embodiment, the invention provides a kit for the diagnosis and/or prognosis of breast cancer, comprising in a first container an polynucleotide that hybridizes to a nucleotide sequence that encodes SEQ ID NO: 3 under stringent conditions, wherein said polynucleotide is at least 3700 nucleotides in length, and further comprising in a second container a known amount of a nucleic acid comprising SEQ ID NO: 2. In another embodiment, the invention provides a kit comprising in one or more containers a forward primer and a reverse primer that amplify at least a portion of the nucleotide sequence of SEQ ID NO: 1 when used in the polymerase chain reaction, wherein said forward primer and said reverse primer are complementary and hybridizable to nucleotides 1-862, 2927-3045, or 3412-3929 of SEQ ID NO: 1 or the complementary sequence thereof. In another embodiment, the invention provides a kit comprising in a container an antibody that binds to a protein the amino acid sequence of which consists of SEQ ID NO: 3, or to a fragment of said protein, and further comprising in a second container a known amount of said protein or a fragment thereof to which said antibody binds. In another embodiment, the invention provides an article of manufacture comprising a container comprising a purified protein comprising SEQ ID NO: 3.

### 5.8.1 Sample Collection

In the present invention, target polynucleotide molecules are extracted from a sample taken from an individual afflicted with breast cancer, or suspected of being afflicted with breast cancer (in a diagnostic scenario). The sample may be collected in any clinically acceptable manner, but must be collected such that marker-derived polynucleotides (i.e., RNA) are preserved. mRNA or nucleic acids derived therefrom (i.e., cDNA or amplified DNA) are preferably labeled distinguishably from standard or control polynucleotide molecules, and both are simultaneously or independently hybridized to a microarray comprising some or all of the markers or marker sets or subsets described above. Alternatively, mRNA or nucleic acids derived therefrom may be labeled with the same label as the standard or control polynucleotide molecules, wherein the intensity of hybridization of each at a particular probe is compared. A sample may comprise any clinically relevant tissue sample, such as a tumor biopsy or fine needle aspirate, or a sample of bodily fluid, such as blood, plasma, serum, lymph, ascitic fluid, cystic fluid, urine or nipple exudate. The sample may be taken from a human, or, in a veterinary context, from non-human animals such as ruminants, horses, swine or sheep, or from domestic companion animals such as felines and canines.

Methods for preparing total and poly(A)+ RNA are well known and are described generally in Sambrook et al., MOLECULAR CLONING: A LABORATORY MANUAL (2nd Ed.), Vols. 1-3, Cold Spring Harbor Laboratory, Cold Spring Harbor, N.Y. (1989)) and Ausubel et al., CURRENT PROTOCOLS IN MOLECULAR BIOLOGY, Vol. 2, Current Protocols Publishing, New York (1994)).

RNA may be isolated from eukaryotic cells by procedures that involve lysis of the cells and denaturation of the proteins contained therein. Cells of interest include wild-type cells (i.e., non-cancerous), drug-exposed wild-type cells, tumor- or tumor-derived cells, modified cells, normal or tumor cell line cells, and drug-exposed modified cells.

Additional steps may be employed to remove DNA. Cell lysis may be accomplished with a nonionic detergent, followed by microcentrifugation to remove the nuclei and hence the bulk of the cellular DNA. In one embodiment, RNA is extracted from cells of the various types of interest using guanidinium thiocyanate lysis followed by CsCl centrifugation to separate the RNA from DNA (Chirgwin et al., Biochemistry 18:5294-5299 (1979)). Poly(A)+ RNA is selected by selection with oligo-dT cellulose (see Sambrook et al., MOLECULAR CLONING: A LABORATORY MANUAL (2nd Ed.), Vols. 1-3, Cold Spring Harbor Laboratory, Cold Spring Harbor, N.Y. (1989). Alternatively, separation of RNA from DNA can be accomplished by organic extraction, for example, with hot phenol or phenol/chloroform/isoamyl alcohol.

If desired, RNase inhibitors may be added to the lysis buffer. Likewise, for certain cell types, it may be desirable to add a protein denaturation/digestion step to the protocol.

For many applications, it is desirable to preferentially enrich mRNA with respect to other cellular RNAs, such as transfer RNA (tRNA) and ribosomal RNA (rRNA). Most mRNAs contain a poly(A) tail at their 3′ end. This allows them to be enriched by affinity chromatography, for example, using oligo(dT) or poly(U) coupled to a solid support, such as cellulose or Sephadex™ (see Ausubel et al., CURRENT PROTOCOLS IN MOLECULAR BIOLOGY, Vol. 2, Current Protocols Publishing, New York (1994). Once bound, poly(A)+ mRNA is eluted from the affinity column using 2 mM EDTA/0.1% SDS.

The sample of RNA can comprise a plurality of different mRNA molecules, each different mRNA molecule having a different nucleotide sequence. In a specific embodiment, the mRNA molecules in the RNA sample comprise at least 100 different nucleotide sequences. More preferably, the mRNA molecules of the RNA sample comprise mRNA molecules corresponding to each of the marker genes. In another specific embodiment, the RNA sample is a mammalian RNA sample.

In a specific embodiment, total RNA or mRNA from cells are used in the methods of the invention. The source of the RNA can be cells of a plant or animal, human, mammal, primate, non-human animal, dog, cat, mouse, rat, bird, yeast, eukaryote, prokaryote, etc. In specific embodiments, the method of the invention is used with a sample containing total mRNA or total RNA from 1×10⁶ cells or less. In another embodiment, proteins can be isolated from the foregoing sources, by methods known in the art, for use in expression analysis at the protein level.

Probes to the homologs of the marker sequences disclosed herein can be employed preferably wherein non-human nucleic acid is being assayed.

### 5.8.2 Determination of DIAPH3 Gene Expression Levels

### 5.8.2.1 General Methods

The expression levels of DIAPH3, and of any other marker genes, in a sample may be determined by any means known in the art. The expression level(s) may be determined by isolating and determining the level (i.e., amount) of nucleic acid transcribed from DIAPH3 and from the other marker genes. Alternatively, or additionally, the level of DIAPH3, alone or in combination with proteins translated from mRNA transcribed from any other marker gene(s), may be determined.

The level of expression of DIAPH3 and other marker genes can be accomplished by determining the amount of mRNA, or polynucleotides derived therefrom, present in a sample. Any method for determining RNA levels can be used. For example, RNA is isolated from a sample and separated on an agarose gel. The separated RNA is then transferred to a solid support, such as a filter. Nucleic acid probes representing one or more markers are then hybridized to the filter by northern hybridization, and the amount of marker-derived RNA is determined. Such determination can be visual, or machine-aided, for example, by use of a densitometer. Another method of determining RNA levels is by use of a dot-blot or a slot-blot. In this method, RNA, or nucleic acid derived therefrom, from a sample is labeled. The RNA or nucleic acid derived therefrom is then hybridized to a filter containing oligonucleotides derived from one or more marker genes, wherein the oligonucleotides are placed upon the filter at discrete, easily-identifiable locations. Hybridization, or lack thereof, of the labeled RNA to the filter-bound oligonucleotides is determined visually or by densitometer. Polynucleotides can be labeled using a radiolabel or a fluorescent (i.e., visible) label.

These examples are not intended to be limiting; other methods of determining RNA abundance are known in the art.

The level of expression of particular marker genes, including DIAPH3, may also be assessed by determining the level of the specific protein expressed from the marker genes. This can be accomplished, for example, by separation of proteins from a sample on a polyacrylamide gel, followed by identification of specific marker-derived proteins using antibodies in a western blot. Alternatively, proteins can be separated by two-dimensional gel electrophoresis systems. Two-dimensional gel electrophoresis is well-known in the art and typically involves isoelectric focusing along a first dimension followed by SDS-PAGE electrophoresis along a second dimension. See, e.g., Hames et al, 1990, GEL ELECTROPHORESIS OF PROTEINS: A PRACTICAL APPROACH, IRL Press, New York; Shevchenko et al., Proc. Nat'l Acad. Sci. U.S.A. 93:1440-1445 (1996); Sagliocco et al., Yeast 12:1519-1533 (1996); Lander, Science 274:536-539 (1996). The resulting electropherograms can be analyzed by numerous techniques, including mass spectrometric techniques, western blotting and immunoblot analysis using polyclonal and monoclonal antibodies.

Alternatively, marker-derived protein levels can be determined by constructing an antibody microarray in which binding sites comprise immobilized, preferably monoclonal, antibodies specific to a plurality of protein species encoded by the cell genome. Preferably, antibodies are present for a substantial fraction of the marker-derived proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array. and their binding is assayed with assays known in the art. Generally, the expression, and the level of expression, of proteins of diagnostic or prognostic interest can be detected through immunohistochemical staining of tissue slices or sections.

Finally, expression of marker genes in a number of tissue specimens may be characterized using a “tissue array” (Kononen et al., Nat. Med 4(7):844-7 (1998)). In a tissue array, multiple tissue samples are assessed on the same microarray. The arrays allow in situ detection of RNA and protein levels; consecutive sections allow the analysis of multiple samples simultaneously.

### 5.8.2.2 Arrays

In preferred embodiments, polynucleotide microarrays are used to measure expression so that the expression status of DIAPH3, alone or in combination with any other breast cancer-related markers, are assessed simultaneously. As used herein, “DIAPH3-derived probe” means a probe the sequence of which is found in DIAPH3, whether in the coding or noncoding region. In a specific embodiment, the invention provides for oligonucleotide or cDNA arrays comprising probes hybridizable to DIAPH3 and to at least five other breast cancer-related markers. In another specific embodiment, the invention provides for oligonucleotide or cDNA arrays comprising probes hybridizable to DIAPH3 and to at least 20 other breast cancer-related markers. In another specific embodiment, the invention provides for oligonucleotide or cDNA arrays comprising probes hybridizable to DIAPH3, wherein said microarray also comprises probes to markers that can distinguish at least one other cancer-related phenotype. In a more specific example, said cancer-related phenotype is ER status (i.e., presence or absence of the estrogen receptor) or BRCA1 status (i.e., whether the breast cancer-associated mutation is in the BRCA1 gene or is sporadic). In another more specific example, said cancer-related phenotype is a phenotype associated with a cancer other than breast cancer. In yet another specific embodiment, the microarray is a commercially-available cDNA microarray that comprises at least one probe the sequence of which is found in DIAPH3. Preferably, such a commercially-available cDNA microarray comprises at least five other breast cancer-related markers. However, such a microarray may, comprise probes derived from 5, 10, 15, 25, 50, 100, 150, 250, 500, 1000 or more breast cancer-related markers, including probes derived from DIAPH3. In a specific embodiment of the microarrays used in the methods disclosed herein, the probes derived from breast cancer-related markers, including DIAPH3-derived probes, make up at least 50%, 60%, 70%, 80%, 90%, 95% or 98% of the probes on the microarray.

General methods pertaining to the construction of microarrays comprising the marker sets and/or subsets above are described in the following sections.

### 5.8.2.2.1 Construction of Microarrays

Microarrays are prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

The probe or probes used in the methods of the invention are preferably immobilized to a solid support which may be either porous or non-porous. For example, the probes of the invention may be polynucleotide sequences which are attached to a nitrocellulose or nylon membrane or filter covalently at either the 3′ or the 5′ end of the polynucleotide. Such hybridization probes are well known in the art (see, e.g., Sambrook et al., MOLECULAR CLONING—A LABORATORY MANUAL (2nd Ed.), Vols. 1-3, Cold Spring Harbor Laboratory, Cold Spring Harbor, N.Y. (1989). Alternatively, the solid support or surface may be a glass or plastic surface. In a particularly preferred embodiment, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel.

In preferred embodiments, a microarray comprises a support or surface with an ordered array of binding (e.g., hybridization) sites or “probes” each representing one of the markers described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). In preferred embodiments, each probe is covalently attached to the solid support at a single site.

Microarrays can be made in a number of ways, of which several are described below. However produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. The microarrays are preferably small, e.g., between 1 cm² and 25 cm², between 12 cm² and 13 cm², or 3 cm². However, larger arrays are also contemplated and may be preferable, e.g., for use in screening arrays. Preferably, a given binding site or unique set of binding sites in the microarray will specifically bind (e.g., hybridize) to the product of a single gene in a cell (e.g., to a specific mRNA, or to a specific cDNA derived therefrom). However, in general, other related or similar sequences will cross hybridize to a given binding site.

The microarrays of the present invention include one or more test probes, each of which has a polynucleotide sequence that is complementary to a subsequence of RNA or DNA to be detected. Preferably, the position of each probe on the solid surface is known. Indeed, the microarrays are preferably positionally addressable arrays. Specifically, each probe of the array is preferably located at a known, predetermined position on the solid support such that the identity (i.e., the sequence) of each probe can be determined from its position on the array (i.e., on the support or surface).

According to the invention, the microarray is an array (i.e., a matrix) in which each position represents one of the markers described herein. For example, each position can contain a DNA or DNA analogue based on genomic DNA to which a particular RNA or cDNA transcribed from that genetic marker can specifically hybridize. The DNA or DNA analogue can be, e.g., a synthetic oligomer or a gene fragment.

### 5.8.2.2.2 Preparing Probes for Microarrays

As noted above, the “probe” to which a particular polynucleotide molecule specifically hybridizes according to the invention contains a complementary genomic polynucleotide sequence. The probes of the microarray preferably consist of nucleotide sequences of no more than 1,000 nucleotides. In some embodiments, the probes of the array consist of nucleotide sequences of 10 to 1,000 nucleotides. In a preferred embodiment, the nucleotide sequences of the probes are in the range of 10-200 nucleotides in length and are genomic sequences of a species of organism, such that a plurality of different probes is present, with sequences complementary and thus capable of hybridizing to the genome of such a species of organism, sequentially tiled across all or a portion of such genome. In other specific embodiments, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, and most preferably are 60 nucleotides in length.

The probes may comprise DNA or DNA “mimics” (e.g., derivatives and analogues) corresponding to a portion of an organism's genome. In another embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates.

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically each probe on the microarray will be between 10 bases and 50,000 bases, usually between 300 bases and 1,000 bases in length. PCR methods are well known in the art, and are described, for example, in Innis et al., eds., PCR PROTOCOLS: A GUIDE TO METHODS AND APPLICATIONS, Academic Press Inc., San Diego, Calif. (1990). It will be apparent to one skilled in the art that controlled robotic systems are useful for isolating and amplifying nucleic acids.

An alternative, preferred means for generating the polynucleotide probes of the microarray is by synthesis of synthetic polynucleotides or oligonucleotides, e.g., using N-phosphonate or phosphoramidite chemistries (Froehler et al., Nucleic Acid Res. 14:5399-5407 (1986); McBride et al., Tetrahedron Lett. 24:246-248 (1983)). Synthetic sequences are typically between about 10 and about 500 bases in length, more typically between about 20 and about 100 bases, and most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., Nature 363:566-568 (1993); U.S. Pat. No. 5,539,083).

Probes are preferably selected using an algorithm that takes into account binding energies, base composition, sequence complexity, cross-hybridization binding energies, and secondary structure (see Friend et al., International Patent Publication WO 01/05935, published Jan. 25, 2001; Hughes et al., Nat. Biotech. 19:342-7 (2001)).

A skilled artisan will also appreciate that positive control probes, e.g., probes known to be complementary and hybridizable to sequences in the target polynucleotide molecules, and negative control probes, e.g., probes known to not be complementary and hybridizable to sequences in the target polynucleotide molecules, should be included on the array. In one embodiment, positive controls are synthesized along the perimeter of the array. In another embodiment, positive controls are synthesized in diagonal stripes across the array. In still another embodiment, the reverse complement for each probe is synthesized next to the position of the probe to serve as a negative control. In yet another embodiment, sequences from other species of organism are used as negative controls or as “spike-in” controls.

### 5.8.2.2.3 Attaching Probes to the Solid Surface

The probes are attached to a solid support or surface, which may be made, e.g., from glass, plastic (e.g., polypropylene, nylon), polyacrylamide, nitrocellulose, gel, or other porous or nonporous material. A preferred method for attaching the nucleic acids to a surface is by printing on glass plates, as is described generally by Schena et al., Science 270:467-470 (1995). This method is especially useful for preparing microarrays of cDNA (See also, DeRisi et al., Nature Genetics 14:457-460 (1996); Shalon et al., Genome Res. 6:639-645 (1996); and Schena et al., Proc. Natl. Acad. Sci. U.S.A. 93:10539-11286 (1995)).

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). When these methods are used, oligonucleotides (e.g., 60-mers) of known sequence are synthesized directly on a surface such as a derivatized glass slide. Usually, the array produced is redundant, with several oligonucleotide molecules per RNA.

Other methods for making microarrays, e.g., by masking (Maskos and Southern, 1992, Nuc. Acids. Res. 20:1679-1684), may also be used. In principle, and as noted supra, any type of array, for example, dot blots on a nylon hybridization membrane (see Sambrook et al., MOLECULAR CLONING: A LABORATORY MANUAL (2nd Ed.), Vols. 1-3, Cold Spring Harbor Laboratory, Cold Spring Harbor, N.Y. (1989)) could be used. However, as will be recognized by those skilled in the art, very small arrays will frequently be preferred because hybridization volumes will be smaller.

In one embodiment, the arrays of the present invention are prepared by synthesizing polynucleotide probes on a support. In such an embodiment, polynucleotide probes are attached to the support covalently at either the 3′ or the 5′ end of the polynucleotide.

In a particularly preferred embodiment, microarrays of the invention are manufactured by means of an ink jet printing device for oligonucleotide synthesis, e.g., using the methods and systems described by Blanchard in U.S. Pat. No. 6,028,189; Blanchard et al., 1996, Biosensors and Bioelectronics 11:687-690; Blanchard, 1998, in SYNTHETIC DNA ARRAYS IN GENETIC ENGINEERING, Vol. 20, J. K. Setlow, Ed., Plenum Press, New York at pages 111-123. Specifically, the oligonucleotide probes in such microarrays are preferably synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes). Microarrays manufactured by this ink-jet method are typically of high density, preferably having a density of at least about 2,500 different probes per 1 cm². The polynucleotide probes are attached to the support covalently at either the 3′ or the 5′ end of the polynucleotide.

### 5.8.2.2.4 Target Polynucleotide Molecules

The polynucleotide molecules which may be analyzed by the present invention (the “target polynucleotide molecules”) may be from any clinically relevant source, but are expressed RNA or a nucleic acid derived therefrom (e.g., cDNA or amplified RNA derived from cDNA that incorporates an RNA polymerase promoter), including naturally occurring nucleic acid molecules, as well as synthetic nucleic acid molecules. In one embodiment, the target polynucleotide molecules comprise RNA, including, but by no means limited to, total cellular RNA, poly(A)+ messenger RNA (mRNA) or fraction thereof, cytoplasmic mRNA, or RNA transcribed from cDNA (i.e., cRNA; see, e.g., Linsley & Schelter, U.S. Pat. No. 6,271,002, or U.S. Pat. Nos. 5,545,522, 5,891,636, or 5,716,785). Methods for preparing total and poly(A)+ RNA are well known in the art, and are described generally, e.g., in Sambrook et al., MOLECULAR CLONING: A LABORATORY MANUAL (2nd Ed.), Vols. 1-3, Cold Spring Harbor Laboratory, Cold Spring Harbor, N.Y. (1989). In one embodiment, RNA is extracted from cells of the various types of interest in this invention using guanidinium thiocyanate lysis followed by CsCl centrifugation (Chirgwin et al., 1979, Biochemistry 18:5294-5299). In another embodiment, total RNA is extracted using a silica gel-based column, commercially available examples of which include RNeasy (Qiagen, Valencia, Calif.) and StrataPrep (Stratagene, La Jolla, Calif.). In an alternative embodiment, which is preferred for S. cerevisiae, RNA is extracted from cells using phenol and chloroform, as described in Ausubel et al., eds., 1989, CURRENT PROTOCOLS IN MOLECULAR BIOLOGY, Vol III, Green Publishing Associates, Inc., John Wiley & Sons, Inc., New York, at pp. 13.12.1-13.12.5). Poly(A)+ RNA can be selected, e.g., by selection with oligo-dT cellulose or, alternatively, by oligo-dT primed reverse transcription of total cellular RNA. In one embodiment, RNA can be fragmented by methods known in the art, e.g., by incubation with ZnCl2, to generate fragments of RNA. In another embodiment, the polynucleotide molecules analyzed by the invention comprise cDNA, or PCR products of amplified RNA or cDNA.

In one embodiment, total RNA, mRNA, or nucleic acids derived therefrom, is isolated from a sample taken from a person afflicted with breast cancer. Target polynucleotide molecules that are poorly expressed in particular cells may be enriched using normalization techniques (Bonaldo et al., 1996, Genome Res. 6:791-806).

As described above, the target polynucleotides are detectably labeled at one or more nucleotides. Any method known in the art may be used to detectably label the target polynucleotides. Preferably, this labeling incorporates the label uniformly along the length of the RNA, and more preferably, the labeling is carried out at a high degree of efficiency. One embodiment for this labeling uses oligo-dT primed reverse transcription to incorporate the label; however, conventional methods of this method are biased toward generating 3′ end fragments. Thus, in a preferred embodiment, random primers (e.g., 9-mers) are used in reverse transcription to uniformly incorporate labeled nucleotides over the full length of the target polynucleotides. Alternatively, random primers may be used in conjunction with PCR methods or T7 promoter-based in vitro transcription methods in order to amplify the target polynucleotides.

In a preferred embodiment, the detectable label is a luminescent label. For example, fluorescent labels, bio-luminescent labels, chemi-luminescent labels, and colorimetric labels may be used in the present invention. In a highly preferred embodiment, the label is a fluorescent label, such as a fluorescein, a phosphor, a rhodamine, or a polymethine dye derivative. Examples of commercially available fluorescent labels include, for example, fluorescent phosphoramidites such as FluorePrime (Amersham Pharmacia, Piscataway, N.J.), Fluoredite (Millipore, Bedford, Mass.), FAM (ABI, Foster City, Calif.), and Cy3 or Cy5 (Amersham Pharmacia, Piscataway, N.J.). In another embodiment, the detectable label is a radiolabeled nucleotide.

In a further preferred embodiment, target polynucleotide molecules from a patient sample are labeled differentially from target polynucleotide molecules of a standard. The standard can comprise target polynucleotide molecules from normal individuals (i.e., those not afflicted with breast cancer). In a highly preferred embodiment, the standard comprises target polynucleotide molecules pooled from samples from normal individuals or tumor samples from individuals having sporadic-type breast tumors. In another embodiment, the target polynucleotide molecules are derived from the same individual, but are taken at different time points, and thus indicate the efficacy of a treatment by a change in expression of the markers, or lack thereof, during and after the course of treatment (i.e., chemotherapy, radiation therapy or cryotherapy), wherein a change in the expression of the markers from a poor prognosis pattern to a good prognosis pattern indicates that the treatment is efficacious. In this embodiment, different timepoints are differentially labeled.

### 5.8.2.2.5 Hybridization to Microarrays

Nucleic acid hybridization and wash conditions are chosen so that the target polynucleotide molecules specifically bind or specifically hybridize to the complementary polynucleotide sequences of the array, preferably to a specific array site, wherein its complementary DNA is located.

Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self complementary sequences.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook et al., MOLECULAR CLONING: A LABORATORY MANUAL (2nd Ed.), Vols. 1-3, Cold Spring Harbor Laboratory, Cold Spring Harbor, N.Y. (1989), and in Ausubel et al., CURRENT PROTOCOLS IN MOLECULAR BIOLOGY, Vol. 2, Current Protocols Publishing, New York (1994). Typical hybridization conditions for the cDNA microarrays of Schena et al. are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Schena et al., Proc. Natl. Acad. Sci. U.S.A. 93:10614 (1993)). Useful hybridization conditions are also provided in, e.g., Tijessen, 1993, HYBRIDIZATION WITH NUCLEIC ACID PROBES, Elsevier Science Publishers B.V.; and Kricka, 1992, NONISOTOPIC DNA PROBE TECHNIQUES, Academic Press, San Diego, Calif.

Particularly preferred hybridization conditions include hybridization at a temperature at or near the mean melting temperature of the probes (e.g., within 5° C., more preferably within 2° C.) in 1M NaCl, 50 mM MES buffer (pH 6.5), 0.5% sodium sarcosine and 30% formamide.

### 5.8.2.2.6 Signal Detection and Data Analysis

When fluorescently labeled probes are used, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously (see Shalon et al., 1996, “A DNA microarray system for analyzing complex DNA samples using two-color fluorescent probe hybridization,” Genome Res. 6:639-645, which is incorporated by reference in its entirety for all purposes). In a preferred embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., Genome Res. 6:639-645 (1996), and in other references cited herein. Alternatively, the fiber-optic bundle described by Ferguson et al., Nature Biotech. 14:1681-1684 (1996), may be used to monitor mRNA abundance levels at a large number of sites simultaneously.

Signals are recorded and, in a preferred embodiment, analyzed by computer, e.g., using a 12 or 16 bit analog to digital board. In one embodiment the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated. The ratio is independent of the absolute expression level of the cognate gene, but is useful for genes whose expression is significantly modulated in association with the different breast cancer-related condition.

### 5.9 Therapeutic Uses of DIAPH3 and DIAPH3

The invention also provides for treatment of breast cancer by administration of a therapeutic compound (termed herein “Therapeutic”). For example, to suppress breast cancer tumor growth or metastasis, a Therapeutic is administered that antagonizes (inhibits) the function of DIAPH3, or of the gene encoding it. Such “Therapeutics” include, but are not limited to, DIAPH3 antagonists, such as antibodies to DIAPH3 or small molecules that disrupt the binding of DIAPH3 to profilin or to a Rho GTPase; or antagonists of DIAPH3 expression, for example, antisense nucleic acids to a nucleic acid encoding DIAPH3. The above is described in detail in the subsections below.

### 5.9.1 DIAPH3 as a Target for Anti-Breast Cancer Drugs

As noted above, DIAPH3 is a formin homology domain protein that contains an FH2 domain. In mouse, an analogous protein, Dia, has been shown to interact with GTPase Rho, a protein that in some cells stimulates the production of stress fibers, which are fibers of actin and myosin that can contract when a cell releases from the substratum. See Ridley, Nature Cell Biol. 1:E64-E67 (1999). When Rho GTPase binds GTP, Rho GTPase interacts with Dia and another protein, ROCK, which is clearly implicated in cytoskeletal rearrangements. See Alberts et al., J. Biol. Chem. 273(15):8616-8622. Dia mediates the formation of stress fibers by recruiting profilin-bound actin to sites where Rho GTPase is active. See Ridley, above. Based on the activities of the related murine Dia protein, DIAPH3 is expected to be a link between one or more human Rho-GTPases and the formation of actin fibers associated with cytoskeletal rearrangements. As such, DIAPH3 is a desirable target for drugs designed to interrupt intracellular signals that direct such rearrangements and detachment from the substratum, leading to metastasis, i.e., anti-cancer drugs.

The invention therefore provides binding agents specific to DIAPH3 and analogs and derivatives thereof, including, without limitation, substrates, agonists, antagonists, and natural intracellular binding targets. For example, novel polypeptide-specific binding agents include DIAPH3 polypeptide-specific receptors, such as somatically recombined polypeptide receptors like specific antibodies or T-cell antigen receptors (see, e.g Harlow and Lane (1988) ANTIBODIES, A LABORATORY MANUAL, Cold Spring Harbor Laboratory) and other natural intracellular binding agents identified with assays such as one-, two- and three-hybrid screens, non-natural intracellular binding agents identified in screens of chemical libraries, etc.

These binding agents may be labeled with fluorescent, radioactive, chemiluminescent, or other easily detectable molecules, either conjugated directly to the binding agent or conjugated to a probe specific for the binding agent. Agents of particular interest modulate DIAPH3 function, e.g., DIAPH3-dependent actin fiber formation; interaction with Rho GTPase or interaction with profilin.

Agents that modulate the interactions of a DIAPH3 with its ligands/natural binding targets can be used to modulate biological processes associated with DIAPH3 function, e.g., by contacting a cell comprising a human diaphanous polypeptide (e.g., administering to a subject comprising such a cell) with such an agent. Biological processes mediated by human diaphanous polypeptides include cellular events that are mediated when DIAPH3 binds a ligand, e.g., cytoskeletal modifications.

Such agents that modulate or inhibit the interaction of DIAPH3 with other cellular components, particularly cellular components involved in DIAPH3-mediated signaling pathways that lead to cytoskeletal rearrangements, are useful as Therapeutics. In particular, such Therapeutics are useful as treatments for cancer and cancer-related conditions, in particular, the treatment of breast cancer.

Methods of assaying for such agents are described in section 5.10, infra.

### 5.9.2 Antisense Regulation of Expression of DIAPH3

The function of the DIAPH3 gene may be inhibited by the use of antisense nucleic acids substantially complementary to the transcript from DIAPH3. The present invention provides the therapeutic or prophylactic use of nucleic acids of at least six nucleotides that are antisense to a gene or cDNA encoding DIAPH3 or a portion thereof. A “DIAPH3 antisense nucleic acid” as used herein refers to a nucleic acid that of hybridizes to a sequence-specific nucleic acid (preferably mRNA) segment (i.e., not the poly-A tract of an mRNA) that encodes DIAPH3, or a portion thereof, by virtue of some sequence complementarity. The antisense nucleic acid may be complementary to a coding and/or noncoding region of an mRNA encoding DIAPH3. Such antisense nucleic acids have utility as Therapeutics that inhibits DIAPH3, and can be used in the treatment of disorders that result from DIAPH3 overexpression.

The antisense nucleic acids of the invention can be oligonucleotides that are double-stranded or single-stranded, RNA or DNA or a modification or derivative thereof, which can be directly administered to a cell, or which can be produced intracellularly by transcription of exogenous, introduced sequences.

The invention further provides pharmaceutical compositions comprising an effective amount of the DIAPH3 antisense nucleic acids of the invention in a pharmaceutically acceptable carrier, as described infra. In another embodiment, the invention is directed to methods for inhibiting the expression of a DIAPH3-encoding nucleic acid sequence in a prokaryotic or eukaryotic cell comprising providing the cell with an effective amount of a composition comprising a DIAPH3 antisense nucleic acid of the invention.

DIAPH3 antisense nucleic acids and their uses are described in detail below.

### 5.9.2.1 DIAPH3 Antisense Nucleic Acids

The DIAPH3 antisense nucleic acids of the present invention are of at least six nucleotides and are preferably longer, typically ranging from 6 to about 50 nucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof, and can be single-stranded or double-stranded. The oligonucleotide can be modified at the base moiety, sugar moiety, or phosphate backbone. The oligonucleotide may include other appending groups such as peptides, or agents facilitating transport across the cell membrane (see, e.g., Letsinger et al., Proc. Natl. Acad. Sci. U.S.A. 86:6553-6556 (1989); Lemaitre et al., Proc. Natl. Acad. Sci. U.S.A. 84:648-652 (1987); U.S. Pat. No. 4,904,582) or blood-brain barrier (see, e.g., PCT Publication No. WO 89/10134, published Apr. 25, 1988), hybridization-triggered cleavage agents (see, e.g., Krol et al., BioTechniques 6:958-976 (1988)) or intercalating agents (see, e.g., Zon, Pharm. Res. 5:539-549 (1988)). In a preferred aspect of the invention, a DIAPH3 antisense oligonucleotide is provided, preferably of single-stranded DNA. In a most preferred aspect, such an oligonucleotide comprises a sequence antisense to the sequence encoding one or more domains of a DIAPH3 protein, most preferably, of a human DIAPH3 protein. The oligonucleotide may be modified at any position on its structure with substituents generally known in the art.

The DIAPH3 antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, 5 beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

In another embodiment, the oligonucleotide comprises at least one modified sugar moiety selected from the group including but not limited to arabinose, 2-fluoroarabinose, xylulose, and hexose.

In yet another embodiment, the oligonucleotide comprises at least one modified phosphate backbone selected from the group consisting of a phosphorothioate, a phosphorodithioate, a phosphoramidothioate, a phosphoramidate, a thiophosphoamidate, a phosphordiamidate, a methylphosphonate, an alkyl phosphotriester, and a formacetal or analog thereof.

In yet another embodiment, the oligonucleotide is an α-anomeric oligonucleotide. An α-anomeric oligonucleotide forms specific double-stranded hybrids with complementary RNA in which, contrary to the usual β-units, the strands run parallel to each other (Gautier et al., Nucl. Acids Res. 15:6625-6641 (1987)).

The oligonucleotide may be conjugated to another molecule, e.g., a peptide, hybridization triggered cross-linking agent, transport agent, hybridization-triggered cleavage agent, etc.

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. Nucl. Acids Res. 16:3209 (1988), methylphosphonate oligonucleotides can be prepared by use of controlled pore glass polymer supports (Sarin et al., Proc. Natl. Acad. Sci. U.S.A. 85:7448-7451 (1988)), etc. In a specific embodiment, the DIAPH3 antisense oligonucleotide comprises catalytic RNA, or a ribozyme (see, e.g., PCT International Publication WO 90/11364, published Oct. 4, 1990; Sarver et al., Science 247:1222-1225 (1990)). In another embodiment, the oligonucleotide is a 2′-O-methylribonucleotide (Inoue et al., Nucl. Acids Res. 15:6131-6148 (1987)), or a chimeric RNA-DNA analog (Inoue et al., FEBS Lett. 215: 327-330 (1987)).

In an alternative embodiment, the DIAPH3 antisense nucleic acid of the invention is produced intracellularly by transcription from an exogenous sequence. For example, a vector can be introduced in vivo such that it is taken up by a cell, within which cell the vector or a portion thereof transcribed, producing an antisense nucleic acid (RNA) of the invention. Such a vector would contain a sequence encoding the DIAPH3 antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the DIAPH3 antisense RNA can be by any promoter known in the art to act in mammalian, preferably human, cells. Such promoters can be inducible or constitutive. Such promoters include but are not limited to: the SV40 early promoter region (Bernoist and Chambon, Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc. Natl. Acad. Sci. U.S.A. 78:1441-1445 (1981)), the regulatory sequences of the metallothionein gene (Brinster et al., Nature 296:39-42 (1982)), etc.

The antisense nucleic acids of the invention comprise a sequence complementary to at least a portion of an RNA transcript of DIAPH3 or a homolog or derivative thereof. However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded DIAPH3 antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA transcribed from a DIAPH3-encoding gene it may contain and still form a stable duplex (or triplex, as the case may be). One skilled in the art can ascertain a tolerable degree of mismatch by use of standard procedures to determine the melting point of the hybridized complex. The antisense nucleic acids of the present invention hybridize to the target nucleic acid under moderately stringent conditions, and more preferably hybridize under highly stringent conditions.

### 5.9.2.2 Therapeutic Use of Antisense Nucleic Acids to DIAPH3

Antisense nucleic acids to the DIAPH3-encoding genes and nucleic acid sequences of the present invention can be used to treat disorders of a cell type that expresses, or preferably overexpresses, DIAPH3. In a specific embodiment, such a disorder is a cancer. In a more specific embodiment, the condition is breast cancer. In a preferred embodiment, a single-stranded DNA antisense DIAPH3 oligonucleotide is used. Cell types which express or overexpress DIAPH3 RNA can be identified by various methods known in the art. Such methods include but are not limited to hybridization with a DIAPH3-specific nucleic acid (e.g. by Northern hybridization, dot blot hybridization, in situ hybridization), observing the ability of RNA from the cell type to be translated in vitro into DIAPH3, immunoassay, etc. In a preferred aspect, primary tissue from a patient can be assayed for expression of DIAPH3 prior to treatment, e.g., by immunocytochemistry or in situ hybridization.

Pharmaceutical compositions of the invention (see Section [5.9.4), comprising an effective amount of a DIAPH3 antisense nucleic acid in a pharmaceutically acceptable carrier, can be administered to a patient having a disease or disorder which is of a type that expresses or overexpresses DIAPH3 or DIAPH3 RNA.

The amount of DIAPH3 antisense nucleic acid which will be effective in the treatment of a particular disorder or condition will depend on the nature of the disorder or condition, and can be determined by standard clinical techniques. Where possible, it is desirable to determine the antisense cytotoxicity of the tumor type to be treated in vitro, and then in useful animal model systems prior to testing and use in humans.

In a specific embodiment, pharmaceutical compositions comprising DIAPH3 antisense nucleic acids are administered via liposomes, microparticles, or microcapsules. In various embodiments of the invention, it may be useful to use such compositions to achieve sustained release of the DIAPH3 antisense nucleic acids. In a specific embodiment, it may be desirable to utilize liposomes targeted via antibodies to specific identifiable tumor antigens (Leonetti et al., 1990, Proc. Natl. Acad. Sci. U.S.A. 87:2448-2451 (1990); Renneisen et al., J. Biol. Chem. 265:16337-16342 (1990)).

### 5.9.3 Other Means of Regulating the Abundance of DIAPH3 RNA

Post-transcriptional gene silencing (PTGS) or RNA interference (RNAi) can also be used to modify RNA abundances, for example, DIAPH3 RNA abundance (Guo et al., 1995, Cell 81:611-620; Fire et al., 1998, Nature 391:806-811). In RNAi, double-stranded RNAs (dsRNAs) known as small interfering RNAs (siRNAs) are injected or transfected into cells to specifically block expression of a homologous gene. In RNAi, both the sense strand and the anti-sense strand can inactivate the corresponding gene. The dsRNAs may be cut by nuclease into 21-23 nucleotide fragments. These fragments may be hybridized to the homologous region of their corresponding mRNAs to form double-stranded segments that are degraded by nuclease (Grant, 1999, Cell 96:303-306; Tabara et al., 1999, Cell 99:123-132; Zamore et al., 2000, Cell 101:25-33; Bass, 2000, Cell 101:235-238; Petcherski et al., 2000, Nature 405:364-368; Elbashir et al., 2001, Nature 411:494-498; Paddison et al., Proc. Natl. Acad. Sci. USA 99:1443-1448). In a preferred embodiment, the siRNA is perfectly complementary to the target mRNA. Therefore, in one embodiment, one or more dsRNAs having sequences homologous to a sequence of human DIAPH3, wherein the abundance of DIAPH3 RNA is to be modified, is transfected into a cell or tissue sample. Any standard method for introducing nucleic acids into cells can be used. In specific embodiments, the interfering RNAs that can be used to modulate the expression of DIAPH3, or a nucleotide sequence encoding DIAPH3, are DIAPH3-1555 and DIAPH3-1805 (see Example 2). Thus, in one embodiment, the invention provides a method of inhibiting the expression of a nucleotide sequence encoding SEQ ID NO: 3 comprising contacting an RNA encoding SEQ ID NO: 3 with an interfering RNA, said interfering RNA comprising a nucleotide sequence complementary and hybridizable to SEQ ID NO: 1, under conditions that allow said interfering RNA and said mRNA to hybridize. In a specific embodiment, the nucleotide sequence of said interfering RNA, or a complement thereof, is present within SEQ ID NO: 1. In another specific embodiment, the nucleotide sequence of said interfering RNA is selected from the group consisting of SEQ ID NO: 274 and SEQ ID NO: 275.

Methods of modifying protein abundances include, inter alia, those altering protein degradation rates and those using antibodies (which bind to proteins affecting abundances of activities of native target protein species). Increasing (or decreasing) the degradation rates of a protein species decreases (or increases) the abundance of that species. Methods for controllably increasing the degradation rate of a target protein in response to elevated temperature and/or exposure to a particular drug, which are known in the art, can be employed in this invention. For example, one such method employs a heat-inducible or drug-inducible N-terminal degron, which is an N-terminal protein fragment that exposes a degradation signal promoting rapid protein degradation at a higher temperature (e.g., 37° C.) and which is hidden to prevent rapid degradation at a lower temperature (e.g., 23° C.) (Dohmen et. al, 1994, Science 263:1273-1276). Such an exemplary degron is Arg-DHFRts, a variant of murine dihydrofolate reductase in which the N-terminal Val is replaced by Arg and the Pro at position 66 is replaced with Leu. According to this method, for example, a gene for a target protein, P, is replaced by standard gene targeting methods known in the art (Lodish et al., 1995, Molecular Biology of the Cell, W.H. Freeman and Co., New York, especially chap 8) with a gene coding for the fusion protein Ub-Arg-DHFRts-P (“Ub” stands for ubiquitin). The N-terminal ubiquitin is rapidly cleaved after translation exposing the N-terminal degron. At lower temperatures, lysines internal to Arg-DHFRts are not exposed, ubiquitination of the fusion protein does not occur, degradation is slow, and active target protein levels are high. At higher temperatures (in the absence of methotrexate), lysines internal to Arg-DHFRts are exposed, ubiquitination of the fusion protein occurs, degradation is rapid, and active target protein levels are low. Heat activation of degradation is controllably blocked by exposure methotrexate. This method is adaptable to other N-terminal degrees which are responsive to other inducing factors, such as drugs and temperature changes.

### 5.9.4 Demonstration of Therapeutic or Prophylactic Utility

The Therapeutics of the invention are preferably tested in vitro, and then in vivo for the desired therapeutic or prophylactic activity, prior to use in humans. For example, in vitro assays which can be used to determine whether administration of a specific Therapeutic is indicated, include in vitro cell culture assays in which a patient tissue sample is grown in culture, and exposed to or otherwise administered a Therapeutic, and the effect of such Therapeutic upon the tissue sample is observed. In one embodiment, a Therapeutic that reverses or reduces formation of actin fibers, such as stress fibers, in, for example, fibroblasts, is selected for therapeutic use in vivo. Assays standard in the art can be used to assess such changes in fiber formation, for example by antibody staining of actin fibers in cells grown in vitro, microscopic examination of the cells to detect changes in morphology, etc.

In various specific embodiments, in vitro assays can be carried out with a patient's breast cancer tumor cells, to determine if a Therapeutic has a desired effect upon such cells.

In another embodiment, breast cancer tumor cells are plated out or grown in vitro, and exposed to a Therapeutic. The Therapeutic that results in a cell phenotype that is more normal (i.e., less representative of a pre-neoplastic state, neoplastic state, malignant state, or transformed phenotype) is selected for therapeutic use. Many assays standard in the art can be used to assess whether a pre-neoplastic state, neoplastic state, or a transformed or malignant phenotype, is present. For example, characteristics associated with a transformed phenotype (a set of in vitro characteristics associated with a tumorigenic ability in vivo) include a more rounded cell morphology, loose substratum attachment relative to normal cells, loss of contact inhibition, loss of anchorage dependence, release of proteases such as plasminogen activator, increased sugar transport, decreased serum requirement, expression of fetal antigens, disappearance of the 250,000 dalton surface protein, etc. (see Luria et al., GENERAL VIROLOGY, 3d ed., John Wiley & Sons, New York pp. 436-446 (1978)).

In other specific embodiments, the in vitro assays described supra can be carried out using a cell line, in particular, a breast cancer cell line, rather than a cell sample derived from the specific patient to be treated.

Compounds for use in therapy can be tested in suitable animal model systems prior to testing in humans, including but not limited to rats, mice, chicken, cows, monkeys, rabbits, etc. For in vivo testing, prior to administration to humans, any animal model system known in the art may be used.

### 5.9.4 Therapeutic/Prophylactic Administration and Compositions

The invention provides methods of treatment (and prophylaxis) by administration to a subject of an effective amount of a Therapeutic of the invention. In a preferred aspect, the Therapeutic is substantially purified. The subject is preferably an animal, including but not limited to animals such as cows, pigs, horses, chickens, cats, dogs, etc., and is preferably a mammal, and most preferably human. In a specific embodiment, a non-human mammal is the subject. Formulations and methods of administration that can be employed can be selected from among those described herein below.

Various delivery systems are known and can be used to administer a Therapeutic of the invention, e.g., encapsulation in liposomes, microparticles, microcapsules, recombinant cells capable of expressing the Therapeutic, receptor-mediated endocytosis (see, e.g., Wu and Wu, J. Biol. Chem. 262:4429-4432 (1987)), construction of a Therapeutic nucleic acid as part of a retroviral or other vector, etc. Methods of introduction include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

In another embodiment, the Therapeutic can be delivered in a vesicle, in particular a liposome (see Langer, Science 249:1527-1533 (1990); Treat et al., in LIPOSOMES IN THE THERAPY OF INFECTIOUS DISEASE AND CANCER, Lopez-Berestein and Fidler (eds.), Liss, N.Y., pp. 317-372, 353-365 (1989))

In yet another embodiment, the Therapeutic can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see MEDICAL APPLICATIONS OF CONTROLLED RELEASE, Langer and Wise (eds.), CRC Pres., Boca Raton, Fla. (1974); CONTROLLED DRUG BIOAVAILABILITY: DRUG PRODUCT DESIGN AND PERFORMANCE, Smolen and Ball (eds.), Wiley, N.Y. (1984); Ranger and Pewas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the thymus, thus requiring only a fraction of the systemic dose (see, e.g., Goodson, in MEDICAL APPLICATIONS OF CONTROLLED RELEASE, supra, vol. 2, pp. 115-138 (1984)). Other controlled release systems are discussed in the review by Langer (Science 249:1527-1533 (1990)).

In a specific embodiment where the Therapeutic is a nucleic acid encoding a protein Therapeutic, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, DuPont), or coating with lipids or cell-surface receptors or transfecting agents, or by administering it in linkage to a homeobox-like peptide which is known to enter the nucleus (see e.g., Joliot et al., Proc. Natl. Acad. Sci. U.S.A. 88:1864-1868 (1991)), etc. Alternatively, a nucleic acid Therapeutic can be introduced intracellularly and incorporated within host cell DNA for expression, by homologous recombination.

The present invention also provides pharmaceutical compositions. Such compositions comprise a therapeutically effective amount of a Therapeutic, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in REMINGTON'S PHARMACEUTICAL SCIENCES by E. W. Martin. Such compositions will contain a therapeutically effective amount of the Therapeutic, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The Therapeutics of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the Therapeutic of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. In one embodiment, the kit provides a container having a therapeutically-active amount of a Therapeutic. Optionally associated with such container(s) can be a notice in the form prescribed by a governmental agency regulating the manufacture, use or sale of pharmaceuticals or biological products, which notice reflects approval by the agency of manufacture, use or sale for human administration.

### 5.10 Screening for DIAPH3 Agonists and Antagonists

DIAPH3 nucleic acids, proteins, and derivatives also have uses in screening assays to detect molecules that specifically bind to DIAPH3 nucleic acids, DIAPH3, or derivatives or analogs thereof and thus have potential use as agonists or antagonists of DIAPH3, in particular, molecules that affect breast cell proliferation, division, detachment from a substrate, etc. In a preferred embodiment, such assays are performed to screen for molecules with potential utility as anti-cancer drugs or lead compounds for drug development. The invention thus provides assays to detect molecules that specifically bind to DIAPH3 nucleic acids, DIAPH3, or derivatives thereof. For example, recombinant cells expressing DIAPH3 nucleic acids can be used to recombinantly produce DIAPH3 in these assays, to screen for molecules that bind to DIAPH3. Molecules (e.g., putative binding partners of DIAPH3) are contacted with DIAPH3 or fragment thereof under conditions conducive to binding, and then molecules that specifically bind to DIAPH3 are identified. Similar methods can be used to screen for molecules that bind to DIAPH3 derivatives or DIAPH3 nucleic acids. Methods that can be used to carry out the foregoing are commonly known in the art.

Thus, in one embodiment, the invention provides method of identifying a molecule that specifically binds to a ligand, comprising contacting a ligand with one or more candidate binding molecules under conditions conducive to binding between said ligand and said molecules, wherein said ligand is selected from the group consisting of a first protein comprising SEQ ID NO: 3, a second protein comprising a fragment of SEQ ID NO: 3 comprising the FH2 domain of DIAPH3 but less than all of SEQ ID NO: 3, and a nucleic acid encoding said first protein or said second protein, comprising (a) contacting said ligand with a plurality of molecules under conditions conducive to binding between said ligand and the molecules; and (b) identifying a molecule within said plurality that specifically binds to said ligand. In various embodiments, said molecule is a protein, for example, an antibody; a nucleic acid; or a small molecule. As used herein, the term “small molecule” includes, but is not limited to, organic or inorganic compounds (i.e., including heteroorganic and organometallic compounds) having a molecular weight less than 10,000 grams per mole, organic or inorganic compounds having a molecular weight less than 5,000 grams per mole, organic or inorganic compounds having a molecular weight less than 1,000 grams per mole, organic or inorganic compounds having a molecular weight less than 500 grams per mole, organic or inorganic compounds having a molecular weight less than 100 grams per mole, and salts, esters, and other pharmaceutically acceptable forms of such compounds. Salts, esters, and other pharmaceutically acceptable forms of such compounds are also encompassed. In a specific embodiment of this method, any of the protein, the candidate binding molecule or the ligand are be purified. The invention also provides a method of identifying an agent that modulates the binding of a protein comprising SEQ ID NO: 3 to a binding partner, comprising contacting said protein and said binding partner with an agent; and measuring an amount of a complex comprising said protein and said binding partner in the presence of said agent, wherein if said amount differs from said amount in the absence of said agent, said agent is identified as an agent that modulates the binding of said protein to said binding partner. In a more specific embodiment, any of the protein comprising SEQ ID NO: 3, the ligand, or the agent are purified.

By way of example, diversity libraries, such as random or combinatorial peptide or nonpeptide libraries can be screened for molecules that specifically bind to DIAPH3. Many libraries are known in the art that can be used, e.g., chemically synthesized libraries, recombinant (e.g., phage display libraries), and in vitro translation-based libraries. Examples of chemically synthesized libraries are described in Fodor et al., Science 251:767-773 (1991); Houghten et al., Nature 354:84-86 (1991); Lam et al., Nature 354:82-84 (1991); Medynski, Bio/Technology 12:709-710 (1994); Gallop et al., J. Medicinal Chemistry 37(9):1233-1251 (1994); Ohlmeyer et al., Proc. Natl. Acad. Sci. U.S.A. 90:10922-10926 (1993); Erb et al., Proc. Natl. Acad. Sci. U.S.A. 91:11422-11426 (1994); Houghten et al., Biotechniques 13:412 (1992); Jayawickreme et al., Proc. Natl. Acad. Sci. U.S.A. 91:1614-1618 (1994); Salmon et al., Proc. Natl. Acad. Sci. U.S.A. 90:11708-11712 (1993); PCT Publication No. WO 93/20242; and Brenner and Lerner, Proc. Natl. Acad. Sci. U.S.A. 89:5381-5383 (1992).

Examples of phage display libraries are described in Scott and Smith, Science 249:386-390 (1990); Devlin et al., Science, 249:404-406 (1990); Christian, R. B., et al., J. Mol. Biol. 227:711-718 (1992)); Lenstra, J. Immunol. Meth. 152:149-157 (1992); Kay et al., Gene 128:59-65 (1993); and PCT Publication No. WO 94/18318 published Aug. 18, 1994. In vitro translation-based libraries include but are not limited to those described in PCT Publication No. WO 91/05058 published Apr. 18, 1991; and Mattheakis et al., Proc. Natl. Acad. Sci. U.S.A. 91:9022-9026 (1994).

By way of examples of nonpeptide libraries, a benzodiazepine library (see e.g., Bunin et al., Proc. Natl. Acad. Sci. U.S.A. 91:4708-4712 (1994)) can be adapted for use. Peptoid libraries (Simon et al., Proc. Natl. Acad. Sci. U.S.A. 89:9367-9371 (1992)) can also be used. Another example of a library that can be used, in which the amide functionalities in peptides have been permethylated to generate a chemically transformed combinatorial library, is described by Ostresh et al., Proc. Natl. Acad. Sci. U.S.A. 91:11138-11142 (1994).

Screening the libraries can be accomplished by any of a variety of commonly known methods. See, e.g., the following references, which disclose screening of peptide libraries: Parmley and Smith, Adv. Exp. Med. Biol. 251:215-218 (1989); Scott and Smith, Science 249:386-390 (1990); Fowlkes et al., Bio/Techniques 13:422-427 (1992); Oldenburg et al., Proc. Natl. Acad. Sci. U.S.A. 89:5393-5397 (1992); Yu et al., Cell 76:933-945 (1994); Staudt et al., Science 241:577-580 (1988); Bock et al., Nature 355:564-566 (1992); Tuerk et al., Proc. Natl. Acad. Sci. U.S.A. 89:6988-6992 (1992); Ellington et al., Nature 355.850-852 (1992); U.S. Pat. No. 5,096,815, U.S. Pat. No. 5,223,409, and U.S. Pat. No. 5,198,346, all to Ladner et al.; Rebar and Pabo, Science 263:671-673 (1993); and PCT Publication No. WO 94/18318, published Aug. 8, 1994.

In a specific embodiment, screening can be carried out by contacting the library members with DIAPH3 (or nucleic acid or analog or derivative thereof) immobilized on a solid phase and harvesting those library members that bind to the protein (or nucleic acid or derivative). Examples of such screening methods, termed “panning” techniques are described by way of example in Parmley and Smith, Gene 73:305-318 (1988); Fowlkes et al., Bio/Techniques 13:422-427 (1992); PCT Publication No. WO 94/18318; and in references cited herein above.

In another embodiment, the two-hybrid system for selecting interacting proteins in yeast (Fields and Song, Nature 340:245-246 (1989); Chien et al., Proc. Natl. Acad. Sci. U.S.A. 88:9578-9582 (1991)) can be used to identify molecules that specifically bind to DIAPH3 or a derivative or analog thereof.

In another embodiment, screening can be carried out by creating a peptide library in a prokaryotic or eukaryotic cells, such that the library proteins are expressed on the cells' surface, followed by contacting the cell surface with DIAPH3 and determining whether binding has taken place. Alternatively, the cells are transformed with a nucleic acid encoding DIAPH3, such that DIAPH3 is expressed on the cells' surface. The cells are then contacted with a potential agonist or antagonist, and binding, or lack thereof, is determined. In a specific embodiment of the foregoing, the potential agonist or antagonist is expressed in the same or a different cell such that the potential agonist or antagonist is expressed on the cells' surface.

### 5.11 Transgenic Animals

The invention also provides animal models. Transgenic animals that have incorporated and express a constitutively-functional DIAPH3 gene, DIAPH3 cDNA, or homolog or derivative thereof, have use as animal models of cancer and/or tumorigenesis. Such animals can be used to screen for or test molecules for the ability to suppress tumorigenesis or breast or other cancer cell proliferation, and thus the ability to treat, ameliorate or prevent such diseases and disorders. In one embodiment, animal models of breast cancer are provided.

In particular, each transgenic line expressing a particular key gene under the control of the regulatory sequences of a characterizing gene is created by the introduction, for example by pronuclear injection, of a vector containing the transgene into a founder animal, such that the transgene is transmitted to offspring in the line. The transgene preferably randomly integrates into the genome of the founder but in specific embodiments may be introduced by directed homologous recombination. In a preferred embodiment, the transgene is present at a location on the chromosome other than the site of the endogenous characterizing gene. In a preferred embodiment, homologous recombination in bacteria is used for target-directed insertion of the key gene sequence into the genomic DNA for all or a portion of the characterizing gene, including sufficient characterizing gene regulatory sequences to promote expression of the characterizing gene in its endogenous expression pattern. In a preferred embodiment, the characterizing gene sequences are on a bacterial artificial chromosome (BAC). In specific embodiments, the key gene coding sequences are inserted as a 5′ fusion with the characterizing gene coding sequence such that the key gene coding sequences are inserted in frame and directly 3′ from the initiation codon for the characterizing gene coding sequences. In another embodiment, the key gene coding sequences are inserted into the 3′ untranslated region (UTR) of the characterizing gene and, preferably, have their own internal ribosome entry sequence (IRES).

The vector (preferably a BAC) comprising the key gene coding sequences and characterizing gene sequences is then introduced into the genome of a potential founder animal to generate a line of transgenic animals. Potential founder animals can be screened for the selective expression of the key gene sequence in the population of cells characterized by expression of the endogenous characterizing gene. Transgenic animals that exhibit appropriate expression (e.g., detectable expression of the key gene product having the same expression pattern within the animal as the endogenous characterizing gene) are selected as founders for a line of transgenic animals.

Animals in which the native DIAPH3 expression is interrupted are also provided. Such animals can be initially produced by promoting homologous recombination between a DIAPH3 gene in its chromosome and an exogenous DIAPH3 gene that has been rendered biologically inactive. Preferably the sequence inserted includes a heterologous sequence, e.g., an antibiotic resistance gene. In a preferred aspect, this homologous recombination is carried out by transforming embryo-derived stem (ES) cells with a vector containing an insertionally inactivated gene, wherein the active gene encodes DIAPH3, such that homologous recombination occurs; the ES cells are then injected into a blastocyst, and the blastocyst is implanted into a foster mother, followed by the birth of the chimeric animal. Such an animal is also called a “knockout animal,” in which DIAPH3 has been inactivated (see Capecchi, Science 244:1288-1292 (1989)). The chimeric animal can be bred to produce additional knockout animals. Chimeric animals can be and are preferably non-human mammals such as mice, hamsters, sheep, pigs, cattle, etc. In a specific embodiment, a knockout mouse is produced.

Such knockout animals are expected to develop or be predisposed to developing diseases or disorders involving T cell underproliferation and thus can have use as animal models of such diseases and disorders, e.g., to screen for or test molecules for the ability to promote activation or proliferation and thus treat or prevent such diseases or disorders.

Knockouts, including tissue-specific knockouts (in which the gene of interest is inactivated in particular tissues), can also be made by methods known in the art. Accordingly, the invention provides a transgenic animal that comprises a recombinant non-human animal in which a gene encoding a protein comprising SEQ ID NO: 3, or a naturally-occurring variant of the same, has been inactivated by a method comprising introducing a nucleic acid into the plant or animal or an ancestor thereof, which nucleic acid or a portion thereof becomes inserted into or replaces said gene, or a progeny of such animal in which said gene has been inactivated.

### 5.12 Imaging

The present invention also provides methods for imaging a portion of a patient, particularly imaging a breast cancer tumor within a breast cancer patient, by administration of a sufficient amount of a labeled antibody of the instant invention, i.e., an antibody that binds specifically to a protein the amino acid sequence of which consists of SEQ ID NO: 3, or a fragment thereof. The antibody is labeled, preferably with a radioisotope. Preferably, the antibody binds detectably to a protein the amino acid sequence of which consists of SEQ ID NO: 3, but not detectably above background to any other protein, although it may bind to other proteins that do not interfere with the imaging results. In a specific embodiment, the antibody binds to an epitope present in amino acids 1110-1152 of SEQ Id NO: 3.

A wide variety of metal ions suitable for in vivo tissue imaging have been tested and utilized clinically, and may be used to label the antibody for imaging purposes. For imaging with radioisotopes, the following characteristics are generally desirable: (a) low radiation dose to the patient; (b) high photon yield which permits a nuclear medicine procedure to be performed in a short time period; (c) ability to be produced in sufficient quantities; (d) acceptable cost; (e) simple preparation for administration; and (f) no requirement that the patient be sequestered subsequently. These characteristics generally translate into the following: (a) the radiation exposure to the most critical organ is less than 5 rad; (b) a single image can be obtained within several hours after infusion; (c) the radioisotope does not decay by emission of a particle; (d) the isotope can be readily detected; and (e) the half-life is less than four days (Lamb and Kramer, “Commercial Production of Radioisotopes for Nuclear Medicine”, In Radiotracers For Medical Applications, Vol. 1, Rayudu (Ed.), CRC Press, Inc., Boca Raton, pp. 17-62). Preferably, the metal is technetium-99.

The targets that one may image include any breast cancer tumor associated with an increase in the expression of the gene encoding the DIAPH3 protein (SEQ ID NO: 3). One may use such labeled antibodies according to the present invention in vivo (e.g., using radiotherapeutic metal complexes) upon administration to a patient, or in vitro (e.g., using a radiometal or a fluorescent metal complex), to diagnose breast cancer, to prognose breast cancer, to assess the progress of a breast cancer, with or without treatment. Such use in vitro may comprise contacting fresh cells obtained directly from a tumor taken from a breast cancer patient, cells that have been frozen and thawed, or cell lines derived from any breast cancer tumor. Thus, in one embodiment, the invention provides a method of imaging a breast cancer tumor, comprising contacting cells of said tumor with an antibody that binds specifically to a protein the amino acid sequence of which consists of SEQ ID NO: 3, wherein said antibody is labeled, and detecting said label. In a specific embodiment, said contacting is performed in vivo in a breast cancer patient. In a more specific embodiment, said imaging is used to support a diagnosis of breast cancer. In another more specific embodiment, said imaging is used to support a prognosis of an individual having breast cancer. In another specific embodiment, said contacting is performed in vitro using breast cancer tumor cells in culture.

A breast cancer tumor may be imaged, for example, by administering to a subject an effective amount of an antibody containing a label in which the label is radioactive, and recording the scintigraphic image of a breast of said subject obtained from the decay of the radioactive metal. Likewise, a magnetic resonance (MR) image of a breast cancer tumor in a subject may be imaged by administering to the subject an effective amount of an antibody composition containing a metal in which the metal is paramagnetic, and recording the MR image of an internal region of the subject.

Other methods include enhancing a sonographic image of an internal region of a subject comprising administering to a subject an effective amount of an antibody containing a metal and recording the sonographic image of an internal region of the subject. In this latter application, the metal is preferably any non-toxic heavy metal ion. A method of enhancing an X-ray image of an internal region of a subject is also provided which comprises administering to a subject an antibody containing a metal, and recording the X-ray image of an internal region of the subject. A radioactive, non-toxic heavy metal ion is preferred.

The antibodies may be linked to a variety of labels. Such labels include, but are not limited to, radioactive substances (e.g. ¹¹¹In, ¹²⁵I, ¹³¹I, ^{99m}Tc, ²¹²B, ⁹⁰Y, ¹⁸⁶Rh); biotin; fluorescent tags; or imaging reagents (e.g. those described in U.S. Pat. No. 4,741,900 and U.S. Pat. No. 5,326,856).

### 6. EXAMPLES

### Example 1

### Full-Length Human DIAPH3 Gene as a Marker for Poor Prognosis of Breast Cancer

A study was undertaken to identify human genes the expression of which differed in breast cancer tumor cells in comparison to non-cancerous cells. The details of these experiments are disclosed in International Publication No. WO 02/103320, published Dec. 27, 2002, entitled “Diagnosis and Prognosis of Breast Cancer Patients,” which is incorporated herein by reference in its entirety. In these experiments, a set of 231 markers was identified whose up-regulation or down-regulation correlated with either good or poor prognosis, where poor prognosis is defined as the development in a patient of a distant metastasis within five years of initial diagnosis.

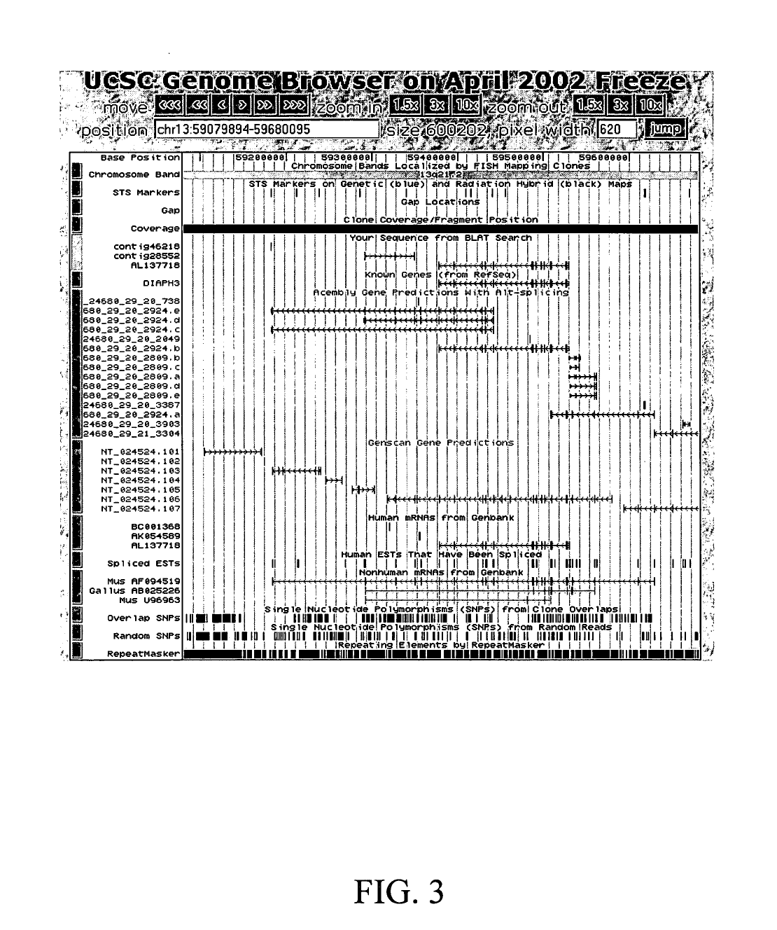

Array data indicated that three of these 231 markers, Contig28552, and Contig46218, and a partial cDNA, AL137718, the expression of each of which is highly correlated with poor prognosis, were overexpressed in poor-prognosis breast cancer patients. AL137718, Contig28552 and Contig46218 are located at the same chromosome locus, 13q21.2, and span about 340 kb. AL137718 lacks a stop codon upstream of the putative starting methionine and its 3′ is also shorter than the mouse ortholog, AF094519, indicating the possibility of additional 5′ and 3′ coding regions. A UCSC BLAT search (available on the Internet at genome-test.cse.ucsc.edu/cgi-bin/hgBlat?hgsid=1719513) revealed an Acembly gene prediction that extended the ORF in both 5′ and 3′ regions of AL137718 and also overlapped with Contig28552. This prediction (Hs13_{—}10007_{—}28_{—}4_t13_Hs13_{—}10007_{—}28_{—}5_{—}494.b; FIG. 3) served as a template for designing RT-PCR and sequencing primers. Additional primers were designed using the Phil Green predicted sequence of Contig46218.

Materials and Methods

A variety of overlapping RT-PCR products was created using a Qiagen One-Step RT-PCR kit (Qiagen, Valencia, Calif.) following the manufacturer's protocol and the primer pairs listed in Table 3. The RT-PCR input RNA was either 5 ng breast adenocarcinoma tRNA (MDA-MB361, Ambion, Inc., Austin, Tex.), or cytoplasmic RNA purified from a human breast-cancer cell line, ZR-75-1 (ATCC, Manassas, Vs.) using RNeasy Midi kit per manufacturer's instructions (Qiagen, Valencia, Calif.). The reactions were cycled in a Gene Amp PCR System 9700 Thermocycler (Applied Biosystems, Foster City, Calif.) as follows: 1) Reverse Transcription, 30 minutes at 50° C.; 2) initial PCR activation step of 15 minutes at 95° C.; 3) 1 minute of denaturation at 94° C., 1 minute of annealing at 68° C., and extension for 1 minute, 45 seconds at 72° C. for 40 cycles; 4) completion with a final extension of 10 minutes at 72° C. 10 μl of the resulting reaction product was electrophoresed on a 1% agarose (Invitrogen, Carlsbad, Calif.) gel stained with 0.5 μg/ml ethidium bromide (Fisher Biotech, Fair Lawn, N.J.). The gel was visualized and photographed with an ultraviolet light box.

3 μl of the RT-PCR product was used in a cloning reaction employing the reagents and instructions provided with the TOPO TA cloning kit (Invitrogen, Carlsbad, Calif.). 2 μl of the cloning reaction was used to transform TOP10 chemically competent Escherichia coli provided with the cloning kit following the manufacturer's instructions. Transformed cells were spread on LB agar plates containing 100 μg/ml Ampicillin (Sigma, St. Louis, Mo.) and 80 μg/ml X-GAL (5-Bromo-4-chloro-3-indoyl-D-galactoside, Sigma, St. Louis, Mo.). Plates were incubated overnight at 37° C. White colonies were picked from the plates and used to seed 2ml cultures of liquid LB medium supplemented with 100 μg/ml Ampicillin. These cultures were incubated overnight at 37° C. in a shaking incubator. Plasmid DNA was extracted from these cultures using the Qiagen (Valencia, Calif.) Qiaquick Spin Miniprep kit following the manufacturer's protocol. 1 μl of each DNA miniprep was digested 1 hour at 37° C. with 1 82 l of the restriction enzyme EcoRI (provided at 10 units/μl by Gibco/Invitrogen, Carlsbad, Calif.). The digestion reaction was electrophoresed on a 1% agarose gel and the DNA bands were visualized and photographed on a UV light box to determine which plasmid clones generated EcoRI fragments of the expected size.

Sequencing reactions used 8 μl of miniprep or PCR product, 4 μl of primer (at 1 μM), and 8 μl of BigDye Terminator Cycle Sequencing Ready Reaction (Applied Biosystems, Foster City, Calif.). Primers used in sequencing are listed in Table 3. PCR sequencing reactions were carried out using Gene Amp PCR System 9700 (Applied Biosystems, Foster City, Calif.) using the PCR conditions in the instructions supplied with the Ready Reaction kit. Sequencing reactions were purified using the DyeEx Spin Kit (Qiagen, Valencia, Calif.) and dried for 20 minutes on low heat in a Speed Vac Plus (SC110A, from Savant, Holbrook, N.Y.) attached to a Universal Vacuum Sytem 400 (also from Savant). The reactions were resuspended in 3 μl of a 6 to 1 mixture of formamide (Sigma, St. Louis, Mo.) with 25 mM EDTA (Sigma) and 50 mg/ml dextran blue (Sigma). The reactions were then heated to 100° C. for 2 minutes and chilled on ice. The DNA was sequenced on an ABI 377 DNA Sequencer. The sequencing gel was prepared using a Long Ranger Singel Pack (BioWhittaker Molecular Applications, Rockland, Me.) according to the manufacturer's instructions. 2 μl of the sequencing reaction were loaded into each well of the gel. The gel was run for 3.5 hours using the 36E 2400 run module, the dye set DT (BD set Any Primer) and the dRHOD Matrix. Sequencing results were analyzed, edited, and compiled into contiguous sequences using the program Sequencher (Gene Codes, Ann Arbor, Mich.).

TABLE 3

Primers used for reverse transcription
or sequencing.

| | | |
|---|---|---|
| | | SEQ ID |
| Primer Name | Primer sequence | NO |

| | | |
|---|---|---|
| M13 Forward (−20) | GTAAAACGACGGCCAGT | 7 |
| M13 Reverse | GGAAACAGCTATGACCATG | 8 |
| MB9 | TAATACGACTCACTATAGGG | 9 |
| DIAPH3_4_2 | GCAGATTATCCATCACTCCTGTCT | 10 |
| PG46218_1 | GAAATTGCAATCCCAAGTTTATTC | 11 |
| PG46218_2 | CATCTTTCTAAGCCACTGGAATTT | 12 |
| DIAPH3_81_F | GACTTCAGCGGTTGGGCTAGGCTG | 13 |
| DIAPH3_2558_R | GCTCAGGTTCACATAAGTTGC | 14 |
| DIAPH3_1831_F | GATTAATGAGCTTCAAGCAGAGC | 15 |
| DIAPH3_2067_F | CCCTGGGATTCCTTGGAGGAC | 16 |
| DIAPH3_2067_R | GTCCTCCAAGGAATCCCAGGG | 17 |
| DIAPH3_1 | TAGATTCTAAAATTGCCCAGAACC | 18 |
| DIAPH3_2_F | ACCTTCGGATTTAACCTTAGCTCT | 19 |
| DIAPH3_2_R | AGAGCTAAGGTTAAATCCGAAGGT | 20 |
| DIAPH3_3_F | ATGAGACACTTTCGAAGTTACACG | 21 |
| DIAPH3_3_R | CGTGTAACTTCGAAAGTGTCTCAT | 22 |
| DIAPH3_4_2 | AGACAGGAGTGATGGATAATCTGC | 23 |
| DIAPH3.e1.130.F | CGGGAGTAAAACCTGTTGTCGA | 24 |
| DIAPH3.e1.218.F | AAAGATGGAACGGCACCAGCC | 25 |
| DIAPH3.e1.381.R | GAAACTTGGGGCGCTTCTCCCC | 26 |
| DIAPH3.e2.517.F | GCAGTGATTGCTCAGCAGCACCTT | 27 |
| DIAPH3.e2.517.R | AAGGTGCTGCTGAGCAATCACTGC | 28 |
| DIAPH3.e3.671.F | CAAAAAAGAAATGGTGATGCAGTA | 29 |
| DIAPH3.e3.671.R | ATGACGTAGTGGTAAAGAAAAAAC | 30 |
| DIAPH3.1296.F | CTTCACATCAGAAATGAATTTATG | 31 |
| DIAPH3.1296.R | CATAAATTCATTTCTGATGTGAAG | 32 |
| DIAPH3.1779.R | CTGAGTTTCTTGGTGGTCGGTAAA | 33 |
| DIAPH3_45_F | GTGGCGGGAGTTTTCAGAT | 34 |
| BG203073_1_F | TGACAGAAGGGTCACGTTCA | 35 |
| BG203073_1_R | TGAACGTGACCCTTCTGTCA | 36 |
| BG203073_2_F | GGATCAAGGCAGCTGAGAAG | 37 |
| BG203073_2_R | CTTCTCAGCTGCCTTGATCC | 38 |
| Contig28552_1F | GGACTGAGACTCTGCCGAAC | 39 |
| Contig28552_1R | GTTCGGCAGAGTCTCAGTCC | 40 |
| Contig28552_2F | CGAGTCTTTCTCGCTCTGCT | 41 |
| Contig28552_2R | AGCAGAGCGAGAAAGACTCG | 42 |
| Contig46218_2_F | TGCATTTGGCAAAGAGAGTG | 43 |
| Contig46218_2_R | CACTCTCTTTGCCAAATGCA | 44 |
| Contig46218_3_R | TGATGATAATGGGGTCACCA | 45 |

Results

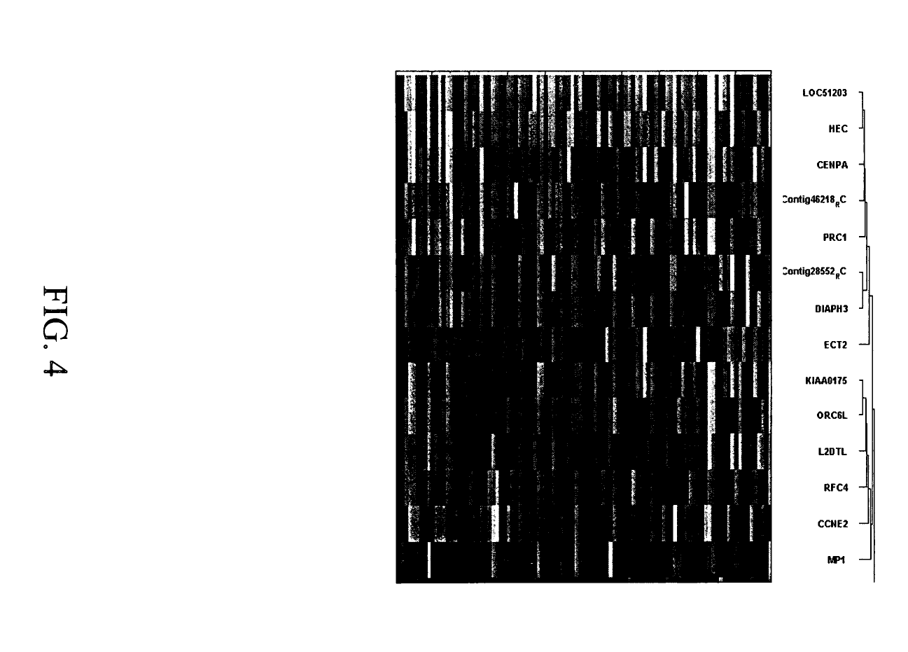

The resulting sequence, named DIAPH3, showed high homology to the mouse diaphanous-related formin protein (Dia2) gene. The sequence of the full-length DIAPH3 cDNA is presented in FIG. 1 (SEQ ID NO: 1). The DIAPH3 protein (SEQ ID NO: 3) contains 1152 amino acid residues, and is predicted to contain an FH2 domain between amino acid residues 636 and 1077. Clustering analysis demonstrated that the three prognosis markers, and therefore DIAPH3, are co-expressed with mitosis-related genes such as human regulator of cytokinesis protein PRC-1 (Jiang et al., Mol. Cell. 2(6):877-85 (1998)), HEC (Chen et al., Mol. Cell Biol. 17(10):6049-6056 (1997)), and ECT2 (Tatsumoto et al., J. Cell Biol. 147(5):921-927 (1999)) (see FIG. 4). This corresponds with DIAPH3's expected role in cytoskeletal rearrangements.

### Example 2

### Effect of Disruption of Human DIAPH3 on Cell Viability and Mitotic Spindle Formation

Materials and Methods

siRNA Transfection in 96-well plates. Small interfering RNA (siRNA) transfection is used to reduce the levels of mRNA for the targeted gene. This lowering of the amount of mRNA can cause lowering of the amount of the protein encoded by the targeted gene. The phenotype of loss of function of a gene can then be determined.

One day prior to transfection, 100 μL of HeLa cells grown in DMEM/10% fetal bovine serum (Invitrogen, Carlsbad, Calif.) to approximately 90% confluency were seeded in a 96-well tissue culture plate (Corning, Corning, N.Y.) at approximately 1500 cells/well. For each transfection 85 μL of OptiMEM (Invitrogen) was mixed with 5 μL siRNA (Dharmacon, Denver, Colo.) from a 20 μM stock. For each transfection 5 μL OptiMEM was mixed with 5 uL Oligofectamine reagent (Invitrogen) and incubated for 5 minutes at room temperature. The 10 μL OptiMEM/Oligofectamine mixture was dispensed into each tube with the OptiMEM/siRNA mixture, mixed and incubated 15-20minutes at room temperature. 10 μL of the transfection mixture was dispensed into each well of the 96-well plate and incubated 4 hrs at 37° and 5% CO₂. After 4 hours, 100 μL/well of DMEM/10% fetal bovine serum was added and the plates were incubated at 37° C. and 5% CO₂ for 72 hours.

Crystal Violet Assay for Cell Growth. Crystal violet stains protein and is used as a measure of the number of cells. 72 hours after transfection with siRNAs, the crystal violet assay was done to determine whether the reduction of DIAPH3 mRNA levels by siRNA results in reduced cell growth and/or increased cell death.

Medium was removed from wells and the cells were washed once with 100 μL/well PBS (Invitrogen). The PBS was removed from the wells and replaced with 100 μL of 100% methanol (Fisher Scientific, Fairlawn, N.J.). The plates were then incubated for approximately 5 minutes at room temperature. The methanol was removed from the wells and the plates were allowed to air dry for approximately 5 minutes. The wells were then stained with 100 μL/well aqueous crystal violet at 0.1% w/v (Sigma, St. Louis, N.J.) for 5 minutes. The stain was removed from the wells and the wells were washed three times in water. 100 μL of 33.3% acetic acid (Fisher Scientific) was added to each well. The plates were incubated 5 minutes at room temperature. The plates were gently agitated to completely mix solubilized stain and the OD of plate at 590 nm was read on the SpectraMax plus plate reader (Molecular Devices, Sunnyvale, Calif.) using Softmax Pro 3.1.2 software (Molecular Devices). The ODs at 590 nM for the DIAPH3 siRNAs were compared to mock treated (no siRNA in the transfection) and luciferase siRNA transfected cells. The OD 590 nM for luciferase was considered to be 100%.

siRNA tranfection in slide chambers. One day prior to transfection, 200 μL of HeLa cells grown in DMEM/10% fetal bovine serum (Invitrogen) to approximately 90% confluency were seeded in an 8-chamber microscope slide (Corning, Corning, N.Y.) at 3000 cells/chamber. For each transfection 85 μL of OptiMEM (Invitrogen) was mixed with 5 μL siRNA (Dharmacon) from a 20 μM stock. For each transfection 5 μL OptiMEM was mixed with 5 μL Oligofectamine reagent (Invitrogen) and incubated 5 minutes at room temperature. The 10 L OptiMEM/Oligofectamine mixture was dispensed into each tube with the OptiMEM/siRNA mixture, mixed and incubated 15-20minutes at room temperature. 15 μL of the transfection mixture was dispensed into each chamber of the 8-chamber slide and incubated 4 hrs at 37° and 5% CO₂. After 4 hours, 100 μL/well of DMEM/10% fetal bovine serum was added and the slides were incubated at 37° and 5% CO₂ for 72 hours.

Staining of slides with anti-α-tubulin antibody and Hoechst dye. 72 hours post transfection, slides were stained with anti-α-tubulin antibody and Hoechst 33342 dye to visualize localization of mitotic spindles and DNA. The medium was removed from the slide chambers and replaced with 200 μL/well of a solution composed of TBST (10 mM Tris-HCL pH 8.0 (Sigma), 150 mM sodium chloride (Sigma), 0.5% Tween20 (Fisher Scientific)), 5 mg/ml BSA (Fisher Scientific) and 2 μL/ml of FITC conjugated α-tubulin antibody (Sigma). The slides were incubated overnight at room temperature and then washed three times with TBST containing 10 μg/ml Hoechst 33342 dye (Sigma). The chambers were incubated 5 minutes in each wash. The TBST/Hoechst washes were followed by 30-minute incubation in PBS. The slides were briefly washed again in PBS. After the removal of the PBS wash, the slide chambers were removed and the slide was allowed to dry. When the slide was dry, a small drop of Flouromount-G (Southern Biotechnology Associates, Inc., Birmingham, Ala.) was added to the slide surface and a coverslip was placed on top. The Flouromount-G was allowed to dry at least 30 minutes before slides were photographed on the Delta Vision Deconvoluting Microscope (Applied Precision, Issaquah, Wash.). Slide photographs were processed using the Delta Vision Sofware.

Results

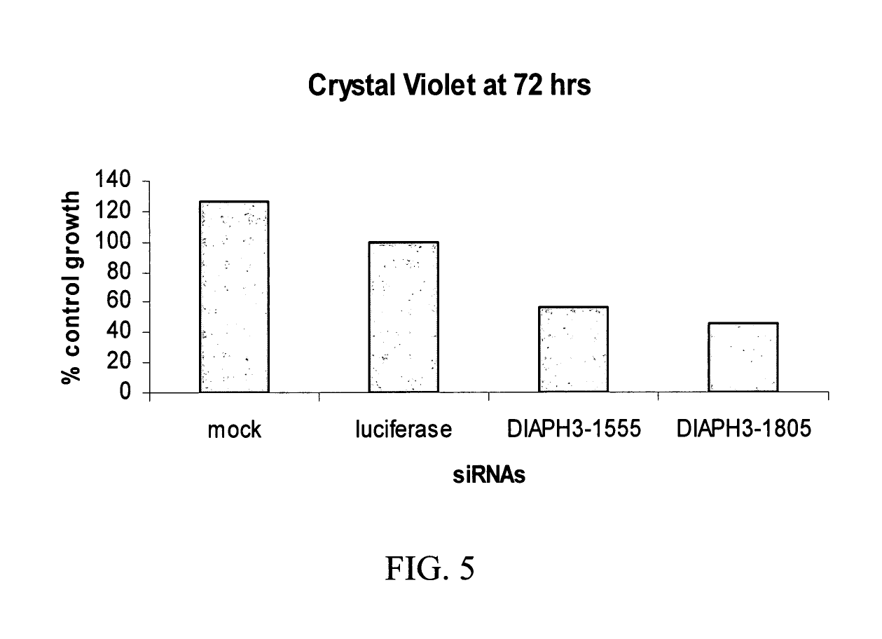

DIAPH3 siRNAs inhibit the growth of cells in cell culture. HeLa cells were transfected with one of two DIAPH3 siRNAs designated DIAPH3-1555 and DIAPH3-1805, an siRNA for luciferase, or were mock-transfected. DIAPH3-1555, an siRNA has the nucleotide sequence GAGUUUACCGACCACCAAGtt (SEQ ID NO: 274). DIAPH3-1805 has the nucleotide sequence UGCGGAUGCCAUUCAGUGGtt (SEQ ID NO: 275). The cells were stained at 72 hours with Crystal Violet, and the number of luciferase siRNA-transfected cells was used as a baseline for determining effects on cell growth. Cells transfected with the DIAPH3-1555 siRNA showed approximately 58%, and cells transfected with DIAPH3-1805 approximately 48% of the amount of Crystal Violet staining shown by luciferase siRNA-transfected cells (FIG. 5). In another experiment, two additional siRNAs, DIAPH3-296 and DIAPH3-2240, showed 92% and 70%, respectively, the level of Crystal Violet staining compared to the luciferase control (data not shown). Thus, DIAPH3 siRNAs are effective at reducing the rate of cell growth.

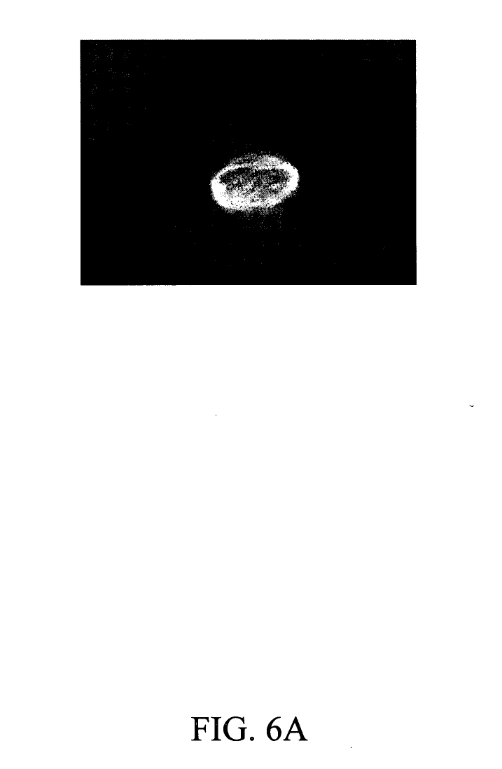

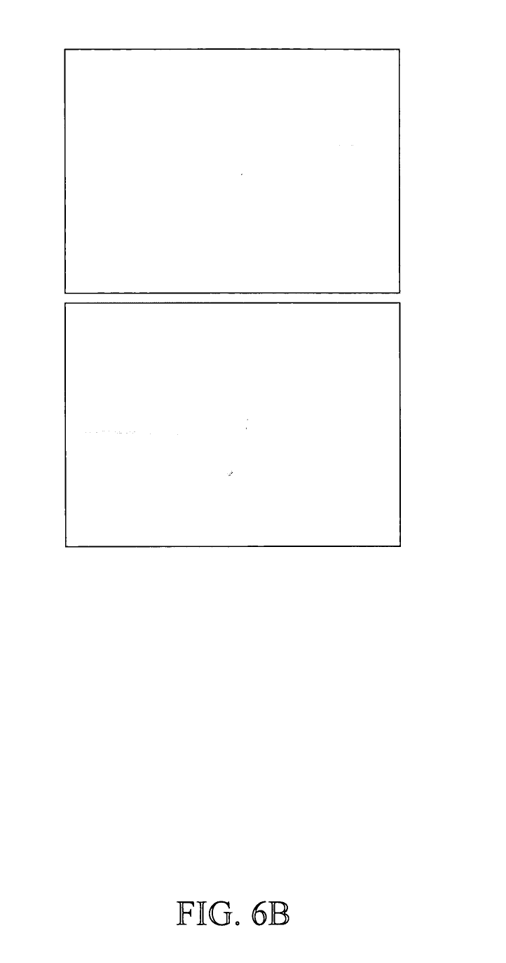

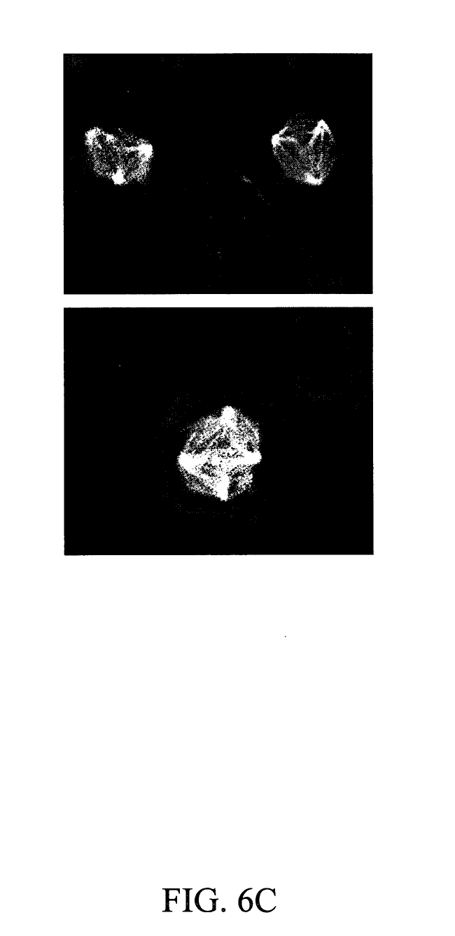

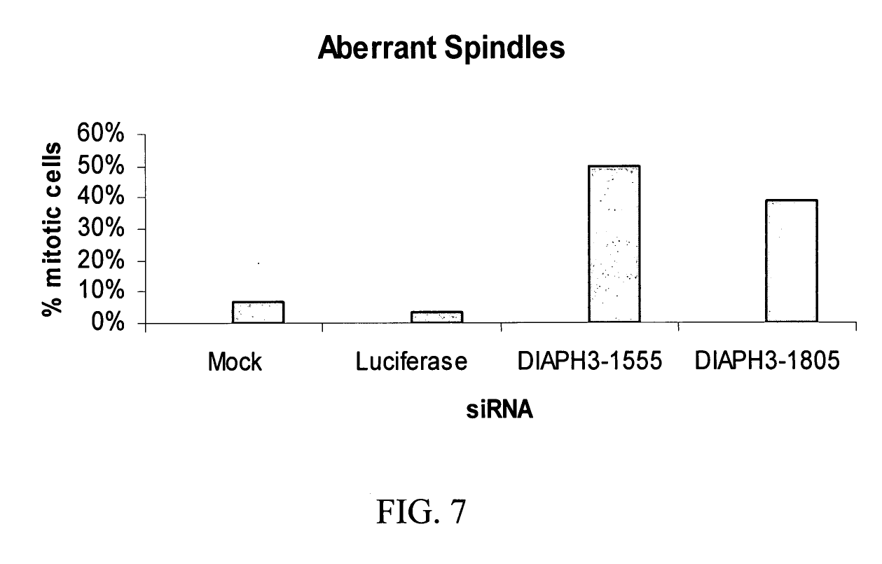

In addition to the effect on cell growth, the DIAPH3 siRNAs cause several striking physiological effects. Most notably, the inhibition of DIAPH3 causes a change in the number of mitotic spindles; rather than the normal two (FIG. 6A), DIAPH3-1555 and DIAPH3-1805 (FIGS. 6B, 6C, respectively) can cause cells to form three or even four mitotic spindles. Treatment of cultures of the cells with DIAPH3 siRNAs resulted in a sharp increase in the number of cells displaying aberrant spindle formation, with approximately 50% of DIAPH3-1555-treated cultures and 39% of DIAPH3-1805-treated cultures displaying aberrant spindles (FIG. 7). In comparison, only approximately 4% of cells in luciferase siRNA control cultures displayed aberrant spindle formation.

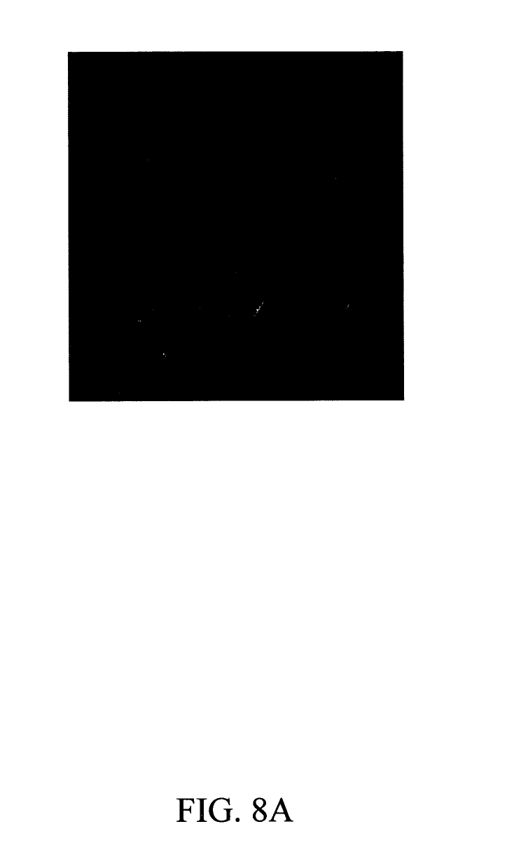

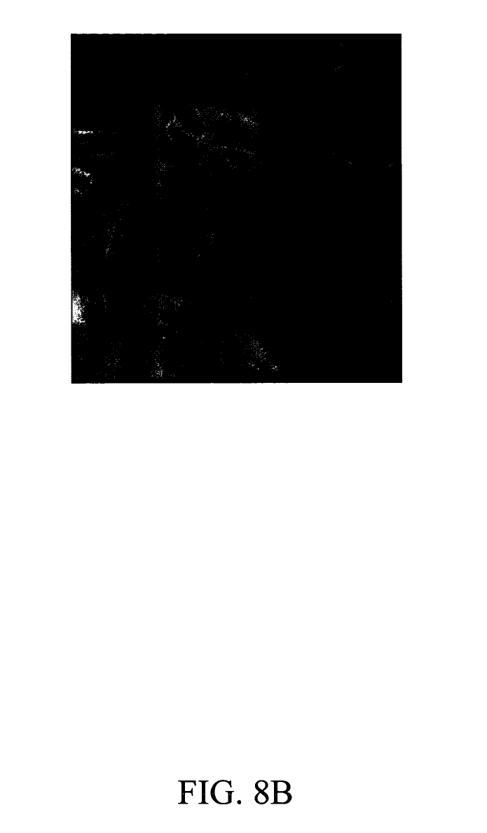

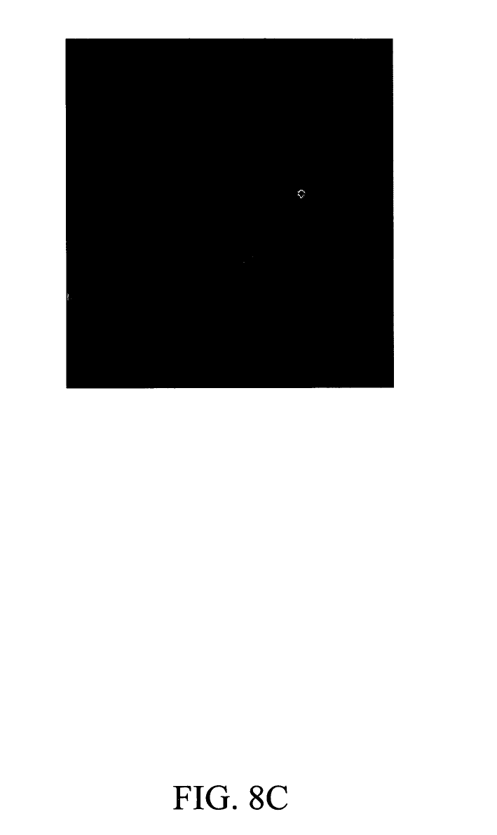

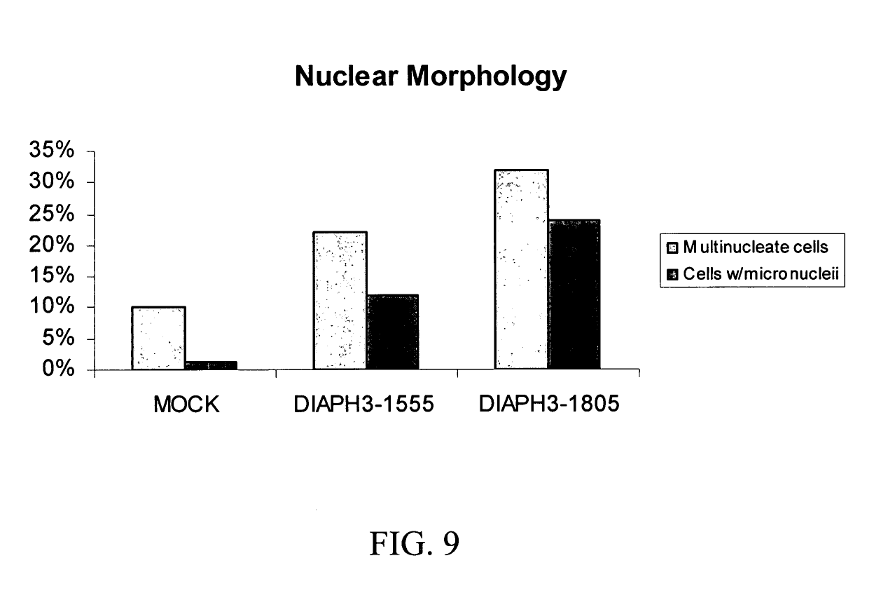

DIAPH3 siRNAs also cause the formation of multinucleate cells (FIGS. 8A-8C) and cells with micronuclei. FIG. 8A depicts control cells transfected with a luciferase reporter gene, showing normal nuclei. In contrast, FIGS. 8B and 8C show multinucleate cells resulting from transfection with siRNA DIAPH3-1805 and DIAPH3-1555, respectively. 22% of DIAPH3-1555-treated cells exhibited multinucleation, and 12% displayed micronucleation, as compared to 10% and 2% for mock-treated cells, respectively (FIG. 9). DIAPH3-1805 cells were even more likely to display multinucleation (32%) or micronucleation (24%) (FIG. 9).

### 7. References Cited

All references cited herein are incorporated herein by reference in their entirety and for all purposes to the same extent as if each individual publication or patent or patent application was specifically and individually indicated to be incorporated by reference in its entirety for all purposes.

Many modifications and variations of the present invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

1. A purified protein comprising the C-terminal 60 contiguous amino acids of SEQ ID NO: 3, wherein said purified protein displays the antigenicity or immunogenicity of SEQ ID NO: 3.

2. The purified protein of claim 1, wherein said protein comprises the C-terminal 500 amino acids of SEQ ID NO: 3.

3. The purified protein of claim 1, wherein said protein comprises SEQ ID NO: 3.

4. The purified protein of claim 1, wherein said protein comprises amino acids 636-1110 of SEQ ID NO: 3.

5. The purified protein of claim 1 that consists of less than the entire amino acid sequence of SEQ ID NO: 3.

6. An isolated nucleic acid comprising 3750 contiguous nucleotides of SEQ ID NO: 1, or the complement thereof.

7. An isolated nucleic acid, wherein said isolated nucleic acid comprises 500 contiguous nucleotides of the 3′ end of SEQ ID NO: 1, or the complement thereof.

8. The isolated nucleic acid of claim 6, wherein said isolated nucleic acid comprises the nucleotide sequence of SEQ ID NO: 1, or the complement thereof.

9. The isolated nucleic acid of claim 6 that is DNA.

10. An isolated nucleic acid comprising a nucleotide sequence encoding the protein of claim 1 or claim 3, or the complement of said nucleotide sequence.

11. A cell transformed with a nucleic acid, said nucleic acid comprising (a) a nucleotide sequence encoding the protein of claim 1, or (b) the complement of said nucleotide sequence.

12. A recombinant cell containing the nucleic acid of claim 6, in which the nucleotide sequence is under the control of a promoter heterologous to the nucleotide sequence.

13. A recombinant cell containing a nucleic acid vector that comprises the nucleic acid of claim 6.

14. An antibody that specifically binds to a protein the amino acid sequence of which consists of SEQ ID NO: 3.

15. The antibody of claim 14 that is monoclonal.

16. A molecule comprising a fragment of the antibody of claim 14, which fragment binds said protein.

17. A method of producing a protein comprising:
growing a recombinant cell containing the nucleic acid of claim 10 in which said nucleotide sequence is under the control of a promoter heterologous to said nucleotide sequence, such that the protein encoded by said nucleic acid is expressed by the cell; and
recovering said expressed protein.

18. An isolated protein that is the product of the process of claim 17.

19. A pharmaceutical composition comprising a therapeutically effective amount of the protein of claim 1, and a pharmaceutically acceptable carrier.

20. A pharmaceutical composition comprising a therapeutically effective amount of the nucleic acid of claim 6; and a pharmaceutically acceptable carrier.

21. A pharmaceutical composition comprising a therapeutically effective amount of the nucleic acid of claim 6; and a pharmaceutically acceptable carrier.

22. A pharmaceutical composition comprising a therapeutically effective amount of the antibody of claim 14, and a pharmaceutically acceptable carrier.

23. A method of identifying an agent that modulates the binding of a protein comprising SEQ ID NO: 3 to a binding partner, comprising contacting said protein and said binding partner with an agent; and measuring an amount of a complex comprising said protein and said binding partner in the presence of said agent, wherein if said amount differs from said amount in the absence of said agent, said agent is identified as an agent that modulates the binding of said protein to said binding partner.

24. The method of claim 23, wherein said agent or said binding partner is purified.

25. A method of identifying a molecule that binds to a ligand, comprising:
(a) contacting a ligand with one or more candidate binding molecules under conditions conducive to binding between said ligand and said molecules, wherein said ligand is selected from the group consisting of a first protein comprising SEQ ID NO: 3, a second protein comprising a fragment of SEQ ID NO: 3 comprising the FH2 domain of DIAPH3 but less than all of SEQ ID NO: 3, and a nucleic acid encoding said first protein or said second protein; and
(b) identifying any of said molecules that specifically binds to said ligand.

26. The method of claim 25, wherein said molecule is an antibody.

27. The method of claim 25, wherein said molecule is a small molecule.

28. A method of diagnosing an individual as having breast cancer, comprising comparing the level of expression of a nucleic acid encoding SEQ ID NO: 3 in a sample derived from breast cells of said individual to a control level of said expression, and diagnosing said individual as having breast cancer if said level of expression of said nucleic acid encoding SEQ ID NO: 3 is higher than said control level of expression.

29. The method of claim 28, wherein said level of expression of a nucleic acid encoding SEQ ID NO: 3 is determined by hybridizing said nucleic acid with an oligonucleotide complementary and hybridizable to nucleotides 1-862, 2927-3045, or 3412-3929 of SEQ ID NO: 1, and determining the amount of said hybridization.

30. A method of diagnosing an individual as having breast cancer comprising comparing the level of a protein the amino acid sequence of which consists of SEQ ID NO: 3 in a sample derived from breast cells of said individual to a control level of said protein; and classifying said individual as having breast cancer if said level of said protein in said sample is higher than said control level of said protein.

31. A method of imaging a breast cancer tumor comprising:
(a) contacting cells of said tumor with an antibody that binds specifically to a protein the amino acid sequence of which consists of SEQ ID NO: 3, wherein said antibody is labeled; and
(b) detecting said label.

32. A method of predicting the prognosis of a breast cancer patient comprising:
(a) determining the level of expression of a nucleic acid encoding SEQ ID NO: 3 in a sample derived from breast cancer tumor cells from said patient;
(b) comparing said level of expression to a control level of said expression; and
(c) predicting that said patient will have a poor prognosis if said level of expression of said nucleic acid encoding SEQ ID NO: 3 in said sample is higher than said control level of said expression.

33. The method of claim 32, wherein said level of expression of a nucleic acid encoding SEQ ID NO: 3 is determined by hybridizing said nucleic acid with an oligonucleotide complementary and hybridizable to nucleotides 1-862, 2927-3045, or 3412-3929 of SEQ ID NO: 1, and determining the amount of said hybridization.

34. The method of claim 32, wherein said determining is carried out by a method comprising:
(a) hybridizing nucleic acids in said sample to an oligonucleotide, wherein said oligonucleotide is hybridizable to SEQ ID NO: 1 or its complement; and
(b) determining the amount of said hybridization.

35. The method of claim 33, wherein said oligonucleotide is a probe on a micro array.

36. The method of claim 33, wherein said oligonucleotide is one of a plurality of probes on a microarray, wherein said plurality comprises probes complementary and hybridizable to nucleic acids respectively encoded by five different breast cancer-related markers that do not encode SEQ ID NO: 3.

37. The method of claim 33, wherein said oligonucleotide is one of a plurality of probes on a microarray, wherein said plurality comprises probes complementary and hybridizable to nucleic acids respectively encoded by twenty different breast cancer-related markers that do not encode SEQ ID NO: 3.

38. The method of claim 36, wherein said five different breast cancer-related markers are present in Table 1.

39. The method of claim 36, wherein said five different breast cancer-related markers are present in Table 2.

40. A method of predicting the prognosis of a breast cancer patient comprising:
(a) determining the level of a protein comprising SEQ ID NO: 3 in a sample derived from breast cancer tumor cells from said patient;
(b) comparing said level of said protein to a control level of said protein; and
(c) predicting that said patient will have a poor prognosis if said level of said protein comprising SEQ ID NO: 3 is significantly higher than said control level of said protein.

41. The method of claim 40, wherein said determining is carried out by a method comprising:
(a) contacting said protein comprising SEQ ID NO: 3 from said sample with an antibody that specifically binds said protein; and
(b) determining the amount of antibody bound to said protein, wherein said amount of antibody bound to said protein indicates said level of said protein in said breast cancer tumor sample.

42. A kit comprising in a first container an oligonucleotide that hybridizes to SEQ ID NO: 1 under stringent conditions, wherein said oligonucleotide is at least 12 nucleotides in length, and wherein said oligonucleotide is complementary and hybridizable to nucleotides 1-862, 2927-3045, or 3412-3929 of SEQ ID NO: 1.

43. A kit for the diagnosis and/or prognosis of breast cancer, comprising in a first container an oligonucleotide that hybridizes to a nucleotide sequence that encodes SEQ ID NO: 3 under stringent conditions, wherein said oligonucleotide is at least 12 nucleotides in length, and wherein said oligonucleotide is complementary and hybridizable to nucleotides 1-862, 2927-3045, or 3412-3929 of SEQ ID NO: 1, and further comprising in a second container a known amount of a nucleic acid to which said oligonucleotide is complementary and hybridizable.

44. The kit of claim 43, wherein said oligonucleotide is a probe on a microarray.

45. The kit of claim 44, wherein said microarray comprises probes complementary and hybridizable to nucleic acids respectively encoded by five breast cancer-related markers other than a nucleotide sequence that encodes SEQ ID NO: 3.

46. An article of manufacture comprising a container comprising a purified protein comprising SEQ ID NO: 3.

47. A kit comprising in a first container an antibody that specifically binds to a protein the amino acid sequence of which consists of SEQ ID NO: 3, or specifically binds to a fragment of said protein, and further comprising in a second container a known amount of said protein or a fragment thereof to which said antibody binds.

48. A kit comprising in one or more containers a forward primer and a reverse primer that amplify at least a portion of the nucleotide sequence of SEQ ID NO: 1 when used in a polymerase chain reaction, wherein said forward primer and said reverse primer are complementary and hybridizable to nucleotides 1-862, 2927-3045, or 3412-3929 of SEQ ID NO: 1 or the complementary sequence thereof.

49. A method of inhibiting the expression of a nucleotide sequence encoding SEQ ID NO: 3 comprising contacting an RNA encoding SEQ ID NO: 3 with an interfering RNA, said interfering RNA comprising a nucleotide sequence complementary and hybridizable to SEQ ID NO: 1, under conditions that allow said interfering RNA and said mRNA to hybridize.

50. The method of claim 49, wherein the nucleotide sequence of said interfering RNA, or a complement thereof, is present within SEQ ID NO: 1.

51. The method of claim 49, wherein the nucleotide sequence of said interfering RNA is selected from the group consisting of SEQ ID NO: 274 and SEQ ID NO: 275.

52. The method of claim 23, wherein, said protein comprising SEQ ID NO: 3 is purified.

53. The method of claim 25, wherein said first protein or said second protein is purified.
